(19) United States
(12) Patent Application Publication
(10) Pub. No.: US 2021/0369946 A1
(43) Pub. Date: Dec. 2, 2021

(54) RADIOEMBOLIZATION DELIVERY DEVICE

(71) Applicant: Bard Peripheral Vascular, Inc., Tempe, AZ (US)

(72) Inventors: Oladipo Peter Akerele-Ale, Tempe, AZ (US); Mircea Despa, Cary, NC (US); Juergen Dorn, Neulußhem (DE); Christopher Dean Drobnik, Wauconda, IL (US); Michael Wesley Drobnik, Downers Grove, IL (US); Casey Tyler Hebert, Tempe, AZ (US); Amanda Kingman, Phoenix, AZ (US); Adam Martin, Holly Springs, NC (US); Alex Palmer, Scottsdale, AZ (US); William Parmentier, Gilbert, AZ (US); Lee Pusateri, Phoenix, AZ (US); Andrew Richards, Durham, NC (US); Brandon David Simmons, Tempe, AZ (US); Clint Solomon, Gilbert, AZ (US); Mark Nicholas Wright, Gilbert, AZ (US); Michael Yarger, Chapel Hill, NC (US)

(73) Assignee: Bard Peripheral Vascular, Inc., Tempe, AZ (US)

(21) Appl. No.: 17/054,552

(22) PCT Filed: May 17, 2019
(86) PCT No.: PCT/US2019/033001

§ 371 Date: Nov. 11, 2020

Related U.S. Application Data
(60) Provisional application No. 62/673,628, filed on May 18, 2018.
(60) Provisional application No. 62/673,632, filed on May 18, 2018.

(51) Int. Cl. A61M 5/00; A61M 5/19; A61N 5/10; A61M 5/178; A61M 5/24

(52) U.S. Cl. CPC A61M 5/007; A61M 5/19; A61N 5/1002; A61M 25/0097; A61M 5/2466; A61M 2209/045; A61M 2205/8206; A61M 5/1785

(57) ABSTRACT

A delivery assembly includes a console including a vial containment region and a vial engagement mechanism extending from the console within the vial containment region. The engagement mechanism is configured to engage a vial assembly. The delivery assembly further includes a sled assembly removably coupled to the console at the vial containment region and a safety shield removably coupled to the console over the vial containment region such that the vial engagement mechanism and the sled assembly are encapsulated within the safety shield when the safety shield is coupled thereto. The sled assembly, the vial assembly, and the safety shield are configured to inhibit radioactive emissions from within the vial containment region.

### CROSS-REFERENCES TO RELATED APPLICATIONS

This application claims the benefit of priority to U.S. Provisional App. No. 62/673,632, entitled “Radioembolization Delivery Device,” filed on May 18, 2018; and U.S. Provisional App. No. 62/673,628, entitled “Dual Stage Syringe,” filed on May 18, 2018, the disclosures of which are incorporated by reference herein.

### TECHNICAL FIELD

The present invention generally relates to medical devices for treating cancer, and more particularly to medical devices configured and operable to deliver radioactive compounds to a treatment area within a patient's body in procedures such as transarterial radioembolization.

### BACKGROUND

In cancer treatments involving radiation therapy, inadvertent or excess exposure to radiation from radioactive therapeutic agents can be harmful and potentially lethal to patients or medical personnel. Accordingly, medical instruments for radiation therapies must be configured to localize the delivery of radioactive material to a particular area of the patient's body while shielding others from unnecessarily being exposed to radiation.

Transarterial Radioembolization is a transcatheter intra-arterial procedure performed by interventional radiology and is commonly employed for the treatment of malignant tumors. During this medical procedure, a microcatheter is navigated into a patient's liver where radioembolizing microspheres loaded with a radioactive compound, such as yttrium-90 (⁹⁰Y), are delivered to the targeted tumors. The microspheres embolize blood vessels that supply the tumors while also delivering radiation to kill tumor cells.

Generally, medical devices for performing radioembolization procedures require multiple syringes, external tubing, a vial containing the radioactive compound, and a bulky shield assembly for containing and shielding the radioactive vial. Such devices typically involve time consuming and labor-intensive setup procedures. The complex devices are commonly stationary and thereby limit a physician's mobility in an operating room to within a certain proximity of the device.

Routine manipulation of a product container storing radioactive material during radioembolization procedures generally requires a Nuclear Medicine Technician, who handles the material with forceps or tweezers. This process involves further potential of exposing additional medical personnel to radioactivity, and contaminating the operating room. Syringes for manually administering the radioactive compound are prone to inconsistent flow rates and pressures. Insufficient injection rates result in decreased bead dispersion, which may impact efficacy of the treatment.

Accordingly, a need exists for a medical device that is configured and operable to perform radioembolization that incorporates a simplistic design and consistent means for administering constant flow rates and pressure of the radioactive compound to the patient's body. A simplified device provides a physician enhanced maneuverability in the operating room during the medical procedure, including an ability to reposition the device about the patient as desired. Additionally, a device with enhanced shielding of the radioactive material enables greater protection to a physician utilizing the medical device while treating a patient.

### BRIEF DESCRIPTION OF THE DRAWINGS

FIG. 1 is a perspective view of a delivery device including a protective shield and handle assembly according to one or more embodiments shown and described herein;

FIG. 2 is a cross-sectional view of the delivery device of FIG. 1 with the handle assembly coupled to the protective shield by a plunger according to one or more embodiments shown and described herein, the cross-section taken along lines 2-2 of FIG. 1;

FIG. 3 is a perspective view of the delivery device of FIG. 1 connected to a syringe and a microcatheter according to one or more embodiments shown and described herein;

FIG. 4A is a partial cross-sectional view of the handle assembly of FIG. 1 in a default position according to one or more embodiments shown and described herein, the cross-section taken along line 4A-4A of FIG. 2;

FIG. 4B is a partial cross-sectional view of the handle assembly of FIG. 1 in an actuated position according to one or more embodiments shown and described herein, the cross-section taken along line 4B-4B of FIG. 2;

FIG. 5 is a perspective view of a handheld delivery device according to one or more embodiments shown and described herein;

FIG. 6 is a partially-exploded perspective view of the handheld delivery device of FIG. 5 including a syringe assembly according to one or more embodiments shown and described herein;

FIG. 7 is a cross-sectional view of the handheld delivery device of FIG. 5 according to one or more embodiments shown and described herein;

FIG. 8 is a perspective view of flushing syringe to be coupled to the handheld delivery device of FIG. 5 according to one or more embodiments shown and described herein;

FIG. 9 is a perspective view of another handheld delivery device according to one or more embodiments shown and described herein;

FIG. 10 is a cross-sectional view of the handheld delivery device of FIG. 9 with multiple syringes received therein, the multiple syringes being manually and electronically actuated, the cross-section taken along line 10-10 of FIG. 9;

FIG. 11 is a perspective view of another handheld delivery device according to one or more embodiments shown and described herein;

FIG. 12 is a cross-sectional view of the handheld delivery device of FIG. 11 with multiple syringes received therein, the multiple syringes being electronically actuated, the cross-section taken along line 12-12 of FIG. 11;

FIG. 13 is a perspective view of a delivery device including a protective shield and a vial sled according to one or more embodiments shown and described herein;

FIG. 14 is a partial perspective view of the delivery device of FIG. 13 including a mechanical assembly according to one or more embodiments shown and described herein;

FIG. 15 is a cross-sectional view of the vial sled of FIG. 13 according to one or more embodiments shown and described herein, the cross-section along line 15-15 of FIG. 13;

FIG. 16 is a perspective view of the vial sled of FIG. 13 with a battery pack removed therefrom according to one or more embodiments shown and described herein;

FIG. 17 is a perspective view of a priming assembly of the vial sled of FIG. 13 according to one or more embodiments shown and described herein;

FIG. 18 is a perspective view of a vial assembly including an engagement head according to one or more embodiments shown and described herein;

FIG. 19A is a perspective view of an alternative engagement head of the vial assembly of FIG. 18 according to one or more embodiments shown and described herein;

FIG. 19B is a perspective view of an alternative engagement head of the vial assembly of FIG. 18 according to one or more embodiments shown and described herein;

FIG. 19C is a perspective view of an alternative engagement head of the vial assembly of FIG. 18 according to one or more embodiments shown and described herein;

FIG. 20 is a partial cross-sectional view of the vial assembly of FIG. 18, the cross-section taken along line 20-20 of FIG. 18;

FIG. 21 is a perspective view of a sterile container assembly according to one or more embodiments shown and described herein;

FIG. 22 is a cross-sectional view of the sterile container assembly of FIG. 21 with the vial assembly of FIG. 18 stored therein according to one or more embodiments shown and described herein, the cross-section taken along line 22-22 of FIG. 21;

FIG. 23 is a perspective view of the delivery device of FIG. 13 with the protective shield removed therefrom and a lever arm of the delivery device actuated according to one or more embodiments shown and described herein;

FIG. 24 is a perspective view of the vial sled of FIG. 13 with the priming assembly of FIG. 17 removed therefrom according to one or more embodiments shown and described herein;

FIG. 25 is a perspective view of the vial sled of FIG. 13 with the vial assembly of FIG. 18 inserted therein according to one or more embodiments shown and described herein;

FIG. 26A is a partial cross-sectional view of the vial assembly of FIG. 18 inserted into the vial sled of FIG. 13 at an initial locking position, with the cross-section taken along line 26-26 of FIG. 25;

FIG. 26B is a partial cross-sectional view of the vial assembly of FIG. 18 inserted into the vial sled of FIG. 13 at a full locking position, with the cross-section taken along line 26-26 of FIG. 25;

FIG. 27 is a partial-perspective view of the vial sled coupled to the delivery device of FIG. 13 with the lever arm coupled to the vial assembly of FIG. 18 according to one or more embodiments shown and described herein;

FIG. 28A is a schematic view of a display interface of the delivery device of FIG. 13 according to one or more embodiments shown and described herein;

FIG. 28B is another schematic view of a display interface of the delivery device of FIG. 13 according to one or more embodiments shown and described herein;

FIG. 29 is a perspective view of the vial sled coupled to the delivery device of FIG. 13, with the lever arm coupled to the vial assembly of FIG. 18 and translated to an extended position according to one or more embodiments shown and described herein;

FIG. 30 is a perspective view of the vial sled of FIG. 13 with the vial assembly of FIG. 18 received therein, with a series of delivery lines coupled to the vial sled according to one or more embodiments shown and described herein;

FIG. 31 is a perspective view of the vial sled coupled to the delivery device of FIG. 13, with the lever arm coupled to the vial assembly of FIG. 18 and translated to a lowered position according to one or more embodiments shown and described herein;

FIG. 32 is a perspective view of the delivery device of FIG. 13 with the protective shield and the vial sled removed therefrom according to one or more embodiments shown and described herein;

FIG. 33 is a flow diagram of an exemplary method of delivering a radioative dose with the delivery device of FIG. 13;

FIG. 34 is a perspective view of an alternative plunger for use with the vial assembly of FIG. 18 according to one or more embodiments shown and described herein;

FIG. 35 is a cross-sectional view of an alternative plunger for use with the vial assembly of FIG. 18 according to one or more embodiments shown and described herein;

FIG. 36A is a cross-sectional view of the plunger of FIG. 35 in a partially extended position relative to the vial assembly of FIG. 18 according to one or more embodiments shown and described herein;

FIG. 36B is a cross-sectional view of the plunger of FIG. 35 in a fully extended position relative to the vial assembly of FIG. 18 according to one or more embodiments shown and described herein;

FIG. 37 is a perspective view of an alternative plunger for use with the vial assembly of FIG. 18 according to one or more embodiments shown and described herein;

FIG. 38A is a perspective view of the plunger of FIG. 37 in a first orientation according to one or more embodiments shown and described herein;

FIG. 38B is a perspective view of the plunger of FIG. 37 in a second orientation according to one or more embodiments shown and described herein;

FIG. 39A is a cross-sectional view of an alternative vial assembly in a first configuration according to one or more embodiments shown and described herein; and

FIG. 39B is a cross-sectional view of the vial assembly of FIG. 39A in a second configuration according to one or more embodiments shown and described herein.

### DETAILED DESCRIPTION

Reference will now be made in detail to various embodiments of delivery devices for administering radioactive compounds to a patient, examples of which are illustrated in the accompanying drawings. Whenever possible, the same reference numerals will be used throughout the drawings to refer to the same or like parts. Directional terms as used herein—for example up, down, right, left, front, back, top, bottom, distal, and proximal—are made only with reference to the figures as drawn and are not intended to imply absolute orientation.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

As used herein, the terms “horizontal,” “vertical,” “distal” and “proximal” are relative terms only, are indicative of a general relative orientation only, and do not necessarily indicate perpendicularity. These terms also may be used for convenience to refer to orientations used in the figures, which orientations are used as a matter of convention only and are not intended as characteristic of the devices shown. The present invention and the embodiments thereof to be described herein may be used in any desired orientation. Moreover, horizontal and vertical walls need generally only be intersecting walls, and need not be perpendicular. As used herein, the singular forms “a,” “an” and “the” include plural referents unless the context clearly dictates otherwise. Thus, for example, reference to “a” component includes aspects having two or more such components, unless the context clearly indicates otherwise.

### I. Mechanical Delivery Device

Referring now to FIGS. 1-4, one embodiment of a delivery device 100 is depicted that is configured and operable to deliver a radioactive material (e.g., radioembolizing beads) while reducing radioactive emissions during use of the delivery device 100. Specifically referring to FIG. 1, the delivery device 100 comprises a base (base plate) 102, a primary housing 110, and a handle assembly 120. Base 102 includes a pair of handles 104 that are configured to facilitate selective positioning of the delivery device 100 during a medical procedure. The base 102 is formed of a radiation shielding material such that any radioactive fluid media stored within the delivery device 100 is effectively shielded from any objects positioned relatively beneath the base 102, thereby minimizing exposure of the radioactive material contained therein. By way of example only, the radiation shielding material of the base 102 may be formed of any combination of plastics, metals, and/or the like. As merely an illustrative example, the base 102 may be formed of acrylonitrile butadiene styrene (ABS), tungsten, pewter, lead, tin, and various other suitable materials configured to inhibit radioactive emissions. The base 102 further includes an elongated member 106 that is configured to provide a mechanical connection point for holding a refilling agent or mixing fluid. By way of example, the elongated member 106 is configured to hold an encasement device, such as a bag and/or syringe filled with one or more fluid mediums therein (e.g., saline, contrast media, etc).

Although the base 102 of the delivery device 100 is shown and described herein as having a squared and/or rectangular shape and defining a planar surface, in other embodiments the base 102 may include various other shapes, sizes, and/or profiles. Additionally, in some embodiments the base 102 may be omitted from the delivery device 100 entirely without departing from the scope of the present disclosure. The primary housing 110 is integral with the base 102 such that the primary housing 110 is fixedly secured to the base 102. Similar to the base 102, the primary housing 110 may be formed of a radiation shielding material that is configured and operable to inhibit radioactive emissions therethrough. As will be described in greater detail below, primary housing 110 is sized and shaped to store radioembolizing beads and/or particles within a central chamber (reservoir) 112 of primary housing 110. In some examples, primary housing 110 may be formed of a clear material that is operable to provide a magnifying effect for enhanced visualization of the radioembolizing beads contained therein. The material of the primary housing 110 may also be shielding to radiation such as beta particles, x-rays, gamma particles, and/or the like. By way of example only, primary housing 110 may be formed of a polycarbonate. In some embodiments, the primary housing 110 may include a viewing window thereon, where the viewing window is formed of a clear material to facilitate a visualization of the contents disposed within primary housing 110. The clear material may be further formed of a radioactive shielding material that is configured and operable to inhibit radioactive emissions therethrough.

Handle assembly 120 is configured to provide a mechanical system for delivering radioembolizing beads from delivery device 100 to a patient. In particular, handle assembly 120 provides a greater range of motion, relative to the handle of a syringe that is proportional to an amount of radioactive material to be delivered to a patient, thereby providing an operator with a more accurate sense of a dose of radioactive material being delivered from the delivery device 100. The configuration and length of handle assembly 120 provides additional distance between the hand of the operator and the radioactive material contained within primary housing 110 to thereby reduce radiation exposure to an operator of the delivery device 100.

Handle assembly 120 comprises a vertical column 122 integral with base 102. Vertical column 122 extends vertically from base 102 such that vertical column 122 is oriented perpendicularly relative to base 102. Handle assembly 120 further comprises an elongated lever 124 having a proximal end 125 and a distal end 126. Elongated lever 124 is pivotably coupled to vertical column 122 at distal end 126 such that proximal end 125 of elongated lever 124 is configured to pivot relative to base 102 about distal end 126. Elongated lever 124 includes a plunger 128 extending toward base 102 from an intermediate junction 130 of elongated lever 124 positioned between proximal end 125 and distal end 126. Plunger 128 is configured to translate relative to base 102 when elongated lever 124 pivots about distal end 126. As will be described in greater detail below, plunger 128 is slidably received within a central chamber 112 of primary housing 110 such that plunger 128 is configured to access the radioactive material contained therein.

Handle assembly 120 further comprises a handle (actuator) 132 that is pivotably coupled to elongated lever 124 at proximal end 125. Handle 132 is sized and shaped to be selectively maneuverable about proximal end 125 of elongated lever 124. In this instance, movement of the handle 132 relative to proximal end 125 is operable to simultaneously pivot handle 132 about elongated lever 124 and pivot the elongated lever 124 about distal end 126. Accordingly, plunger 128 is configured to translate relative to primary housing 110 and base 102 in response to elongated lever 124 pivoting about distal end 126. Thus, actuation of handle 132 is operable to translate plunger 128 into primary housing 110. Although not shown, it should be understood that plunger 128 may include a plurality of markings along a longitudinal length of plunger 128 to provide visual feedback of a displacement of plunger 128 relative to primary housing 110. Additionally or alternatively, in some versions, the delivery device 100 may include a sensor (e.g., linear encoder) that detects or measure linear movement of plunger 128 into central chamber 112. In some versions, delivery device 100 may comprise a locking mechanism configured to engage plunger 128 to thereby releasably fix the plunger 128 at a position relative to primary housing 110.

In some versions, movement of handle assembly 120 may be automated with a stepper or motor (not shown) to facilitate reproducible flow rates, volumes, or other process parameters. With automated movement of the handle assembly 120, an operator can operate the delivery device 100 hands-free, thereby further reducing potential radiation exposure and potential for human error.

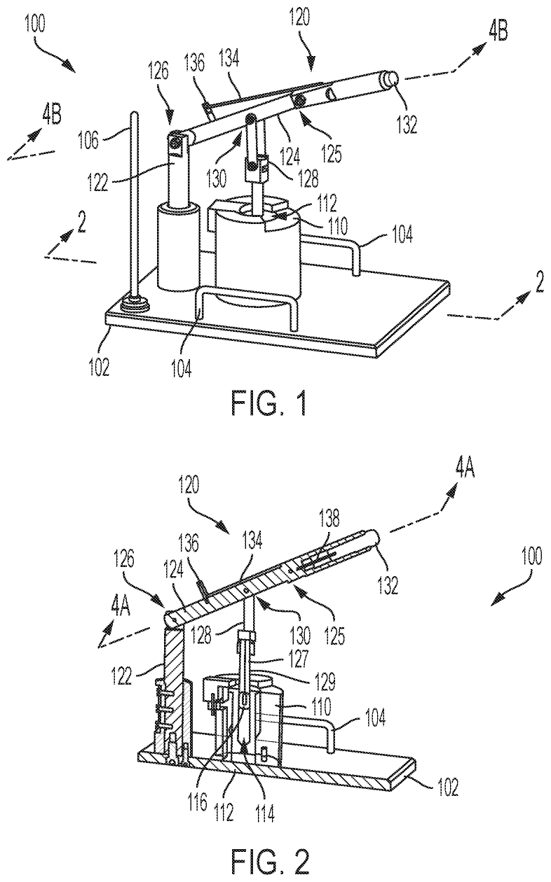

Referring still to FIG. 1, elongated lever 124 includes a marker 134 attached to handle 132 and an interface display 136 attached to elongated lever 124 adjacent to distal end 126. Marker 134 and interface display 136 are cooperatively configured to generate a visual feedback to an operator indicating real time information pertaining to a flow rate administered by delivery device 100. In the embodiment of FIG. 1, interface display 136 includes a series of indicators and is configured to correspond a deflection of handle 132 to either an amount of force or a range of delivery flow rate of the radioactive material. As evident in the view of FIG. 2, with handle 132 oriented parallel relative to elongated lever 124, interface display 136 extends toward a bottom portion of marker 134 such that interface display 136 is positioned adjacent a portion of interface display 136 that represents an acceptable degree of deflection of handle 132, and thus an acceptable flow rate. In other embodiments, the interface display 136 may comprise a scale, a ruler, a digital display, a remote smart device, a tablet, and/or the like.

In contrast, referring back to FIG. 1, with handle 132 oriented transversely relative to elongated lever 124, interface display 136 extends toward a top portion of marker 134 such that interface display 136 is positioned adjacent a portion of interface display 136 that represents an excess degree of deflection of handle 132, i.e. an unacceptable flow rate. As merely an illustrative example only, the series of indicators on interface display 136 may comprise indicia such as a plurality of colors (e.g., green, yellow, orange, red, etc.), a plurality of numbers (e.g., 1 through 5), or other measurable indicia as will be apparent to those of ordinary skill in the art. Alternatively, in other versions delivery device 100 may include an accelerometer or displacement sensor, in lieu of or in addition to marker 134 and interface display 136, such that the accelerometer or displacement sensor is configured to correspond a deflection of handle 132 to either an amount of force or a range of delivery flow rate of the radioactive material.

In the delivery device 100 of FIG. 2, the primary housing 110 including central chamber 112 that is sized and shaped to slidably receive plunger 128 therein. As briefly described above, plunger 128 is configured to translate through central chamber 112 of primary housing 110 in response to actuation (i.e. pivot) of handle 132 about distal end 126 of elongated lever 124. Plunger 128 includes a needle 129 disposed therein. The needle 129 is configured to slidably translate within central chamber 112 as plunger 128 translates relative to primary housing 110. As further seen in FIG. 2, primary housing 110 includes a vial compartment 114 at a bottom end of central chamber 112. Vial compartment 114 is sized and shaped to store therapeutic particles (e.g., radioembolizing beads, radioactive particles, microspheres, etc.) therein. Vial compartment 114 is isolated from the remaining portion of central chamber 112 by a protective seal 116 disposed therein between vial compartment 114 and the remainder of central chamber 112. Thus, the therapeutic particles disposed within vial compartment 114 are not in fluidic communication with the remaining portions of primary housing 110, because protective seal 116 is configured to generate a protective barrier between central chamber 112 and vial compartment 114.

Although not shown, it should be understood that base 102 may further comprise a quick release mechanism positioned beneath primary housing 110. In particular, the quick release mechanism may be sized and shaped to remove vial compartment 114 from within central chamber 112 of primary housing 110.

Needle 129 is configured to puncture the protective seal 116 in response to translation of plunger 128 through central chamber 112. In this instance, access to the therapeutic particles within vial compartment 114 is established when handle 132 of elongated lever 124 is pivoted about distal end 126 to an extent corresponding to the displacement between needle 129 and protective seal 116. Additionally, although not shown, it should be understood that plunger 128 may also include a sterile barrier mechanism proximate to needle 129 that is configured to sterilize the area of contact between needle 129 and protective seal 116. In this instance, the sterile barrier mechanism is operable to minimize potential contamination of protective seal 116 when needle 129 contacts protective seal 116 to access the therapeutic particles within vial compartment 114.

By way of example only, the sterile barrier mechanism may comprise a removable Tyvek® disk. With the sterile barrier mechanism positioned proximate to protective seal 116, the necessity to wipe needle 129 with alcohol prior to advancing needle 129 into vial compartment 114 is removed. Needle 129 includes a plurality of side holes (not shown) along the longitudinal length of needle 129. The side holes (not shown) are configured to generate turbulence within vial compartment 114 as needle 129 extends therein, thereby mixing the therapeutic particles contained therein. The side holes of needle 129 provide access to a central lumen 127 of needle 129 that extends along a longitudinal length of needle 129. As described in greater detail herein, the central lumen 127 of needle 129 is configured to receive a fluid medium (e.g., saline) from a fluid reservoir fluidly coupled thereto such that the fluid medium is transferred into the vial compartment 114 via the plurality of side holes as the needle 129 translates downward into the central chamber 112 of the primary housing 110 in response to generating a positive pressure therein.

Although not shown, it should be understood that vial compartment 114, plunger 128, and/or needle 129 may include plurality of outwardly protruding flaps, outwardly protruding ribs, or other outwardly protruding features configured to further promote the mixture of the radioembolizing beads and the fluid medium as needle 129 and plunger 128 are advanced into vial compartment 114. Additionally or alternatively, delivery device 100 may further include a stir bar (not shown) that is operable to enhance mixing of the radioembolizing beads and the fluid medium within vial compartment 114.

In some versions, delivery device 100 may include a plurality of abutments (not shown) within central chamber 112 of primary housing 110. The plurality of abutments may extend into central chamber 112 and be configured to releasably engage plunger 128 as plunger 128 is translated through central chamber 112 to thereby generate a plurality of stopping points. In this instance, the plurality of abutments temporarily inhibit advancement of plunger 128 into primary housing 110 to thereby provide a tactile feedback to an operator for managing dose control. The tactile feedback experienced at the plurality of stopping points indicate to an operator of the displacement of plunger 128 relative to primary housing 110, thereby informing the operator of the sphere concentration, flow rate, and/or a torque or pressure to be delivered by delivery device 100. In other examples, delivery device 100 may include a stir bar (not shown) within vial compartment 114 and/or central chamber 112 to promote mixing of the radioembolizing beads and the fluid medium received therein.

Referring still to FIG. 2, elongated lever 124 further includes a torque coupling member 138 disposed within elongated lever 124 and handle 132 such that torque coupling member 138 extends between elongated lever 124 and handle 132. In other words, torque coupling member 138 is configured to couple proximal end 125 of elongated lever 124 to handle 132. In the present example, torque coupling member 138 is a resiliently biased spring that is configured to bias handle 132 in a substantially parallel orientation relative to a longitudinal length of elongated lever 124, as is evident in FIG. 2. In this instance, releasing handle 132 returns handle 132 to a default position in parallel orientation with elongated lever 124 such that torque coupling member 138 is operable to suspend plunger 128 in a retracted position relative to central chamber 112 and withdraw needle 129 from contacting the protective seal 116. As will be described in greater detail below, torque coupling member 138 is configured to resist lateral movement of handle 132 toward base 102 such that a predetermined force is required to actuate handle 132.

Torque coupling member 138 provides volumetric flow rate, or alternatively volume speed control, during delivery of the radioactive material from delivery device 100 to a patient. In particular, torque coupling member 138 correlates a deflection of the handle 132 to a flow rate generated by the delivery device 100. In other versions, it should be understood that torque coupling member 138 may be configured to bias the handle 132 in a substantially transverse orientation relative to a longitudinal length of elongated lever 124, as seen in FIG. 1. In this instance, releasing handle 132 returns handle 132 to a default position in transverse orientation with the elongated lever 124 such that torque coupling member 138 is operable to advance the plunger 128 into an extended position relative to central chamber 112 with needle 129 punctured through protective seal 116.

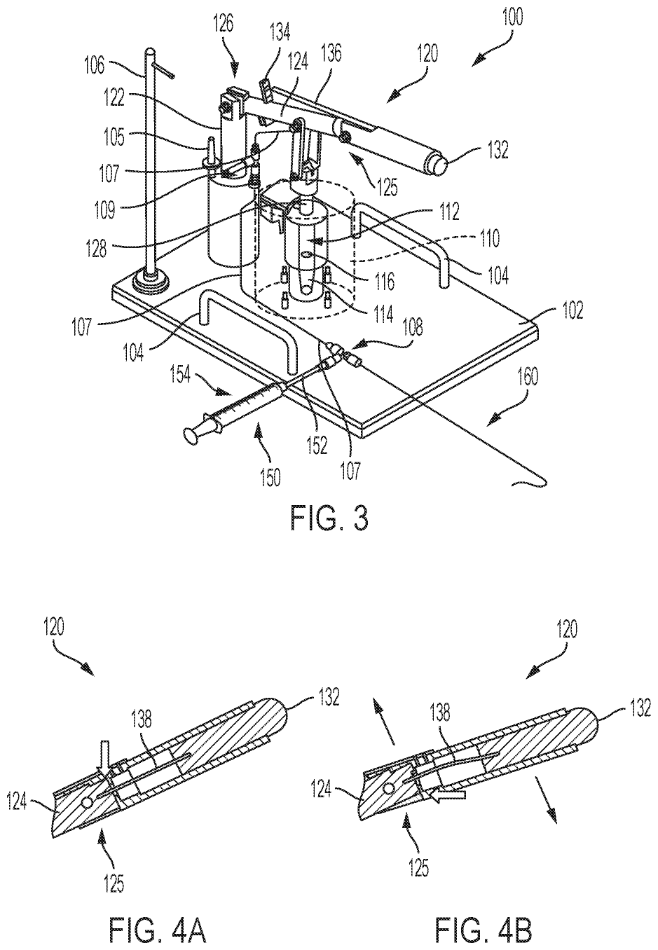

Operation of the delivery device 100 will now be described with reference to FIG. 3. In particular, an operator selectively positions delivery device 100 in an operating room adjacent to a patient by maneuvering the base 102 via handles 104. With delivery device 100 positioned at a desired location, an operator couples a contrast syringe 150, optionally, and a catheter 160 to the delivery device 100 via a first connector valve 108. In particular, first connector valve 108 is a three-way check valve (also known as a T-valve connector) such that a contrast line 152 is connected to contrast syringe 150 at one end and first connector valve 108 at an opposite end. In the present example, contrast syringe 150 includes a contrast medium stored therein, however, it should be understood that contrast syringe 150 may include various other fluid media as will be apparent to those of ordinary skill in the art. Contrast syringe 150 further comprises a plurality of markings 154 along the body of the contrast syringe 150 to thereby indicate to an operator a current volume of contrast medium stored therein. Although not shown, it should be understood that contrast syringe 150 may be coupled to a syringe pump or power injector that is configured to automate the actuation of contrast syringe 150. In this instance, delivery of the contrast medium stored in the contrast syringe 150 may be administered at reliable and consistent flow rates.

Catheter 160 is similarly coupled to first connector valve 108 such that contrast syringe 150 is in fluidic communication with catheter 160. In this instance, delivery device 100 is coupled to first connector valve 108 via a delivery line 107 that is connected to first connector valve 108 at one end and to a second connector valve 109 at an opposite end. In the present example, catheter 160 is a microcatheter sized and shaped to intravenously establish fluidic communication between a target treatment site and delivery device 100. Similar to first connector valve 108, second connector valve 109 is a three-way check valve (also known as a T-valve connector) such that delivery line 107 from first connector valve 108 is coupled to second connector valve 109 at a first end and a fluid reservoir line 105 is attached thereon at another end.

Fluid reservoir line 105 is coupled to a fluid reservoir (not shown) that may comprise a bag or chamber configured to store a fluid medium therein. In the present example, the fluid reservoir contains saline or a contrast medium therein. By way of example only, the fluid reservoir is configured to store an intravenous sugar solution, such as dextrose solution (D5W). It should also be understood that delivery lines 107 and connector valves 108, 109 are sized and shaped to include smooth diameter transitions or interfaces at their intersection points to thereby minimize dead volumes and the potential for sphere settling in the tubing system.

Needle 129 is similarly coupled to second connector valve 109 (see FIG. 3) via another delivery line 107 such that needle 129 of delivery device 100 establishes fluidic communication with contrast syringe 150, catheter 160, and fluid reservoir line 105. The central lumen 127 of needle 129 may be coupled to delivery line 107 such that needle 129 is in fluidic communication with second connector valve 109. In this instance, fluid reservoir line 105 is in communication with central lumen 127 of needle 129 via the second connector valve 109 such that central lumen 127 is operable to receive a fluid medium from the fluid reservoir (not shown) attached to fluid reservoir line 105.

As will be described in greater detail below, advancement of needle 129 downward through central chamber 112 of primary housing 110 generates a negative pressure through the central lumen 127 due to a downward translation of the needle 129. In this instance, the delivery line 107 fluidly coupled to the central lumen 127, which provides fluid communication between the central lumen 127 and the fluid reservoir line 105 via the second connector valve 109 coupled therebetween, causes a negative pressure to similarly be generated within the delivery line 107 and the second connector valve 109. A fluid medium stored within a fluid reservoir (not shown) that is coupled to the fluid reservoir line 105 is drawn from the fluid reservoir and through fluid reservoir line 105 as a result of the negative pressure generated by the needle 129 and transferred to the fluid reservoir by the lines 105, 107 and second connector valve 109 fluidly coupled therebetween. Accordingly, the fluid medium is transferred through into central lumen 127 via second connector valve 109 and delivery line 107. It should be understood that the delivery line 107 extending from the second connector valve 109 extends through a top end of the plunger 128 and into a top end of the central lumen 127 of the needle 129. With the needle 129 slidably received within the central chamber 112 of the primary housing 110 and the delivery line 105 fluidly coupled to the central lumen 127 of the needle 129, the delivery line 105 is effectively in fluid communication with central chamber 112 of the primary housing 110.

In exemplary treatment procedures for expelling the radioembolizing beads, an operator may actuate delivery device 100 by exerting a downward force onto handle 132 relative to base 102 to thereby pivot handle 132 about proximal end 125 and pivot the elongated lever 124 about distal end 126. As briefly described above, torque coupling member 138 is resiliently biased to inhibit downward movement of handle 132 toward base 102 such that handle 132 is biased toward a parallel configuration with elongated lever 124, as best seen in FIG. 4A. In this instance, an operator may apply a predetermined force onto handle 132 to overcome the resilient bias of torque coupling member 138 and thereby actuate elongated lever 124, as seen in FIG. 4B. Application of a consistent force onto handle 132 may overcome the resilient bias of torque coupling member 138 thereby slidably translating the plunger 128 downward through central chamber 112 of primary housing 110.

Needle 129 is already penetrated through protective seal 116 and in fluidic communication with the radioembolizing beads contained in vial compartment 114 such that the downward translation of plunger 128 advances needle 129 toward a bottom surface of vial compartment 114. As briefly described above, prior to a fluid medium being suctioned into central lumen 127, needle 129 is advanced upward relative to vial compartment 114 thereby generating a negative pressure therein by the actuation of handle 132. In this instance, the fluid medium is effectively dispersed from a fluid reservoir and to central lumen 127 of needle 129 as needle 129 translates upward relative to the bottom surface of vial compartment 114. Effectively, the bottom surface of vial compartment 114 serves as a refill starting point for delivery device 100.

The plurality of side holes along needle 129 provide for mixing the fluid medium received within the central lumen 127 into the therapeutic particles (e.g., radioembolizing beads) stored within the vial compartment 114. By exerting a downward force, with vial compartment 114 now in fluidic communication with central lumen 127 of needle 129, the fluid medium is effectively expelled from central lumen 127 of needle 129 and into vial compartment 114 in response to a positive pressure being generated therein when the needle 129 translates relatively downward through the central chamber 112. The radioembolizing bead concentration per delivery cycle can be defined depending on the chosen refill volume. In this instance, plunger 128 is lowered through central chamber 112 together with needle 129 into vial compartment 114. The mixture of therapeutic particles (e.g., radioembolizing beads) and fluid medium, collectively referred to as a suspension fluid or liquid, is thereby injected through central lumen 127 and toward first connector valve 108 via the interconnected system of second connector valve 109 and delivery lines 107.

With needle 129 fully advanced into vial compartment 114 (i.e. the refill starting point), handle 132 is ready for refilling delivery device 100. Handle 132 is lifted up relative to base 102 and remains in a default orientation where handle 132 is substantially parallel with the longitudinal length of elongated lever 124 due to an end stop (not shown) present in the pivot region between handle 132 and elongated lever 124. In this instance, the function of torque coupling member 138 is bypassed. Alternatively, handle 132 may simply be released such that the downward force applied onto handle 132 is removed. In this instance, the resilient bias of torque coupling member 138 returns handle 132 to the default orientation where handle 132 is substantially parallel with the longitudinal length of elongated lever 124. In this instance, plunger 128 is retracted through central chamber 112 thereby withdrawing needle 129 from vial compartment 114. Retraction of plunger 128 and needle 129 generates a negative pressure within vial compartment 114 such that the mixture of radioembolizing beads and fluid medium is extracted through central lumen 127 and toward first connector valve 108 via the interconnected system of second connector valve 109 and delivery lines 107.

As the mixture medium is being transferred toward first connector valve 108, an operator may actuate the contrast syringe 150 to thereby transfer a contrast medium through contrast line 152 and toward first connector valve 108 thereby mixing the various media together at first connector valve 108 prior to delivery to catheter 160. An operator may repeatedly actuate handle 132 to continue filling and flushing a mixture of radioembolizing beads, the fluid medium, and/or a contrast medium into catheter 160 by the pressurization means described above.

### II. Manual Handheld Delivery Device

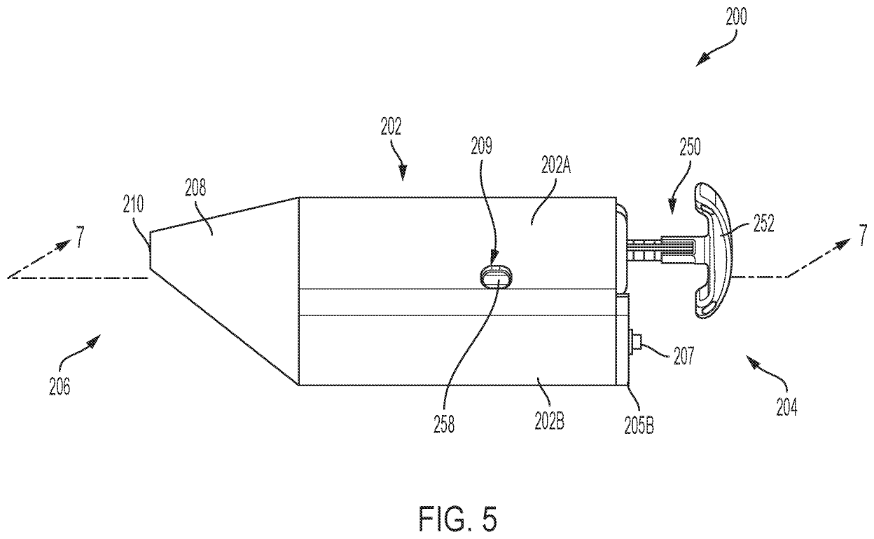

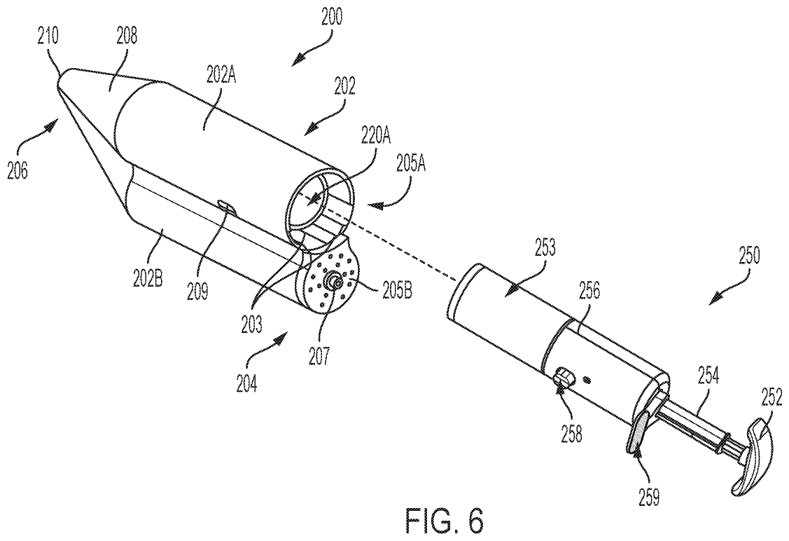

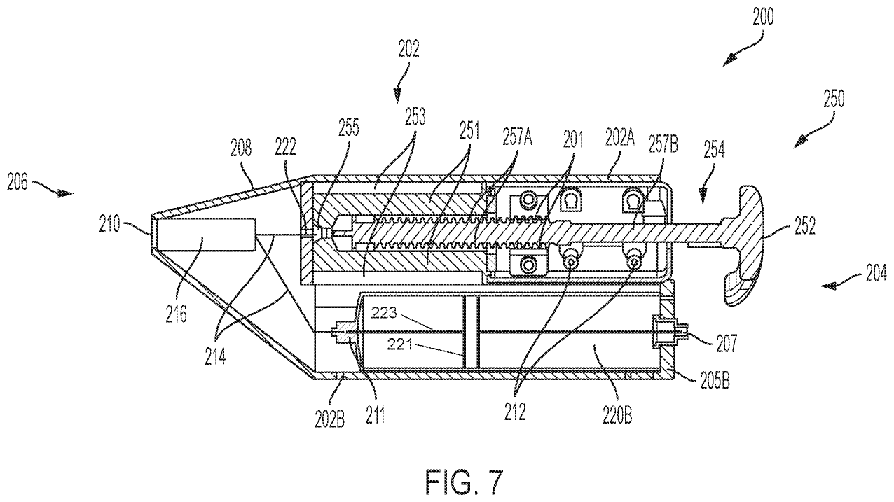

FIGS. 5-7 show another embodiment of a delivery device 200 configured and operable to deliver a radioactive material (e.g., radioembolizing beads) while reducing radioactive emissions during use of the delivery device 200. Referring specifically to FIG. 5, the delivery device 200 comprises a housing 202 extending between a proximal end 204 and a distal end 206. The housing 202 includes a pair of chambers 202A, 202B disposed therein, and in particular at least one chamber 202A that defines an internal cavity 220A (See FIG. 6) that is sized and shaped to receive a device therein, and at least one chamber 202B that defines another internal cavity 220B (see FIG. 7) for storing a fluidic substance therein (e.g., saline). In some embodiments, the chamber 202B may be formed of a translucent material such that the fluidic substance stored within the internal cavity 220B may be visible from an exterior of the delivery device 200. In other embodiments the internal cavity 220B of the second chamber 202B is sized and shaped to receive a device therein, such as, for example, an external fluid reservoir.

In particular, the internal cavity 220A of the chamber 202A is sized and shaped to receive a vial assembly 250 within the delivery device 200. It should be understood that the internal cavity 220A of the chamber 202A may include one or more retention mechanisms that are configured to selectively lock the vial assembly 250 to the delivery device 200 such that vial assembly 250 is securely retained within the internal cavity 220A during use of the delivery device 200. Actuation of the retention mechanism may provide for a selective removal of the vial assembly 250 from the internal cavity 220A of the chamber 202A such that after use of the delivery device 200 the vial assembly 250 may be disposed of separate from the delivery device 200.

In the present example, the retention mechanism of the delivery device 200 comprises an aperture 209 positioned along the housing 202, and in particular, disposed through the chamber 202A. The aperture 209 of the delivery device 200 is sized and shaped to receive a corresponding retention mechanism of the vial assembly 250. In particular, the corresponding retention mechanism of the vial assembly 250 comprises a depressible button 258 such that the aperture 209 receives the depressible button 258 when the vial assembly 250 is slidably received through the internal cavity 220A of the chamber 202A. As will be described in greater detail herein, the depressible button 258 is configured to resiliently expand outward from the vial assembly 250 in response to an alignment of the depressible button 258 with the aperture 209 as the vial assembly 250 is translated through the chamber 202A. In other embodiments, the retention mechanisms of the internal cavity 220A may be configured to permanently secure the vial assembly 250 to the delivery device 200 such that the vial assembly 250 is not subsequently removable from the internal cavity 220A of the chamber 202A. In this instance, the delivery device 200 is disposable together with the vial assembly 250.

Still referring to FIG. 5, with the vial assembly 250 fully positioned within the internal cavity 220A of the chamber 202A, a handle 252 of the vial assembly 250 extends proximally from the housing 202 at the proximal end 204, such that vial assembly 250 is not fully contained within the internal cavity 220A of the chamber 202A. In this instance, the handle 252 of the vial assembly 250 is accessible to an operator of the delivery device 200 when the vial assembly 250 is fully assembled in the delivery device 200. As will be described in greater detail below, with the handle 252 of the vial assembly 250 extending outwardly from and accessible at the proximal end 204 of the delivery device 200, the vial assembly 250 may be actuated by an operator while the vial assembly 250 is securely received within the internal cavity 220A of the chamber 202A. The housing 202 further includes a distal head 208 that is integrally formed with the pair of chambers 202A, 202B of the housing 202. The distal head 208 includes a tapered profile relative to an elongated profile of the pair of chambers 202A, 202B. In particular, the distal head 208 tapers distally toward the distal end 206 of the delivery device 200 to a catheter hub 210 of the delivery device 200. Alternatively, in other embodiments the catheter hub 210 may include tubing and/or standard connections configured to couple the delivery device 200 to various devices.

The catheter hub 210 is configured to couple the delivery device 200 to a device, such as, for example, a catheter (not shown), to thereby facilitate fluidic communication between the delivery device 200 and the device. For example, the catheter hub 210 may comprise a luer fitting that is selectively engageable with a corresponding luer fitting of a device (e.g., a catheter) to thereby couple the delivery device 200 to the device at the catheter hub 210. It should be understood that the internal cavities 220A, 220B of the chambers 202A, 202B of the delivery device 200 may comprise various other sizes and shapes than those shown and described herein to accommodate additional devices (e.g., the vial assembly 250) and/or fluid medias therein without departing from the scope of the present disclosure.

Still referring to FIG. 5, the housing 202 is further sized and shaped to accommodate the maneuverability of the delivery device 200 such that the delivery device 200 is configured to be grasped by an operator. Additionally and/or alternatively, the housing 202 of the delivery device 200 may be sized and shaped to accommodate a corresponding dock and/or holding fixture. The housing 202 of the delivery device 200 may be overmolded with various materials, such as, for example, silicone, thermoplastic elastomers, thermoplastic vulcanizates, and the like. In some embodiments, the housing 202 may include, or be constructed of, a radiation shielding material such that any radioactive material contained within the delivery device 200 is sealed therein, such that exposure to radiation emissions from any radioactive material stored therein are limited to the housing 202. By way of example only, the radiation shielding material of housing 202 may include any combination of plastics and metal. As merely an illustrative example, the housing 202 may be formed of acrylonitrile butadiene styrene (ABS), lead, tungsten, tin, pewter, or other suitable materials configured and operable to inhibit radiation emissions.

Alternatively, it should be understood that the housing 202 may be formed of other material that is not configured to shield against radiation emissions. In this instance, the delivery device 200 may include additional features that are configured to suppress radiation emissions from within the housing 202 of the delivery device 200. For example, the delivery device 200 may include one or more radiation shield inserts positioned within the internal cavities 220A, 220B of the pair of chambers 202A, 202B and/or the distal head 208 to thereby reduce radiation exposure from within the housing 202. By way of example only, the one or more radiation shield inserts may be formed of acrylonitrile butadiene styrene (ABS), lead, tungsten, tin, pewter, or other suitable materials configured and operable to inhibit radiation emissions. Additionally or alternatively, in some versions the delivery device 200 may be over molded with a radioactive shielding material. As merely an illustrative example, this material may comprise silicone, thermoplastic elastomer, thermoplastic vulcanizates, or other suitable materials configured and operable to inhibit radiation emissions.

The vial assembly 250 may be formed of a material comprising plastic, thermoplastic polymers, polycarbonate, polyethylene, polyethylene terephthalate, and the like. As will be described in greater detail herein, in some embodiments at least a portion of the vial assembly 250 that is removably received within the housing 202 of the delivery device 200 may be formed of a material and/or includes features (e.g., a protective shield 253, see FIG. 7) configured and operable to inhibit radiation exposure from a substance stored therein. In this instance, the housing 202 of the delivery device 200 may be formed of a plastic.

Referring now to FIG. 6, the delivery device 200 is depicted with the vial assembly 250 removed from within the internal cavity 220A of the chamber 202A. In particular, the housing 202 of the delivery device 200 includes an opening 205A at the proximal end 204 of the chamber 202A for receiving the vial assembly 250 therethrough. In this instance, the opening 205A is sized and shaped to receive the vial assembly 250 such that the vial assembly 250 encloses the internal cavity 220A of the chamber 202A when received therein. The vial assembly 250 includes a handle 252, a plunger 254, and an elongated body 256 and a depressible button 258 extending laterally outward from the elongated body 256. As briefly described above, the depressible button 258 is resiliently biased to an expanded position and is selectively depressible in response to a compression of the depressible button 258 by a predetermined force. In other words, actuation of the depressible button 258 provides for a depression of the depressible button 258 into the elongated body 256 of the vial assembly 250. Accordingly, the depressible button 258 is configured to resiliently expand outward from the elongated body 256 of the vial assembly 250 upon terminating application of the predetermined force thereon.

The depressible button 258 is sized and shaped to be received through the aperture 209 of the housing 202 such that, in response to an alignment of the depressible button 258 with the aperture 209, the depressible button 258 expands outwardly from the elongated body 256 and extends through the aperture 209. In this instance, the vial assembly 250 is effectively coupled to the housing 202 of the delivery device 200 and securely disposed within the internal cavity 220A of the chamber 202A. It should be understood that in other embodiments the vial assembly 250 may comprise additional depressible buttons 258 along the elongated body 256 for securing the vial assembly 250 to the housing 202 of the delivery device 200. Alternatively, in other embodiments the vial assembly 250 may include other suitable retention mechanisms that are configured and operable to attach the vial assembly 250 to the delivery device 200. As will be described in greater detail herein, in some embodiments actuation of the depressible button 258, and/or other buttons or mechanisms, may facilitate an actuation of the handle 252 and the plunger 254 for administering a dose from the delivery device 200. In this instance, the depressible button 258 further serves as a safety feature in addition to a retention mechanism. As will be described in greater detail herein, in some embodiments the delivery device 200 may include one or more sensors disposed thereon, including, for example, a linear encoder. In this instance, the linear encoder may be disposed over and/or coupled to the plunger 254 such that the plunger 254 extends through the linear encoder and the linear encoder translates simultaneously with the plunger 254.

Still referring to FIG. 6, the chamber 202B of the housing 202 is sized and shaped to receive a fluid medium therein, and in particular, the chamber 202B serves as a fluid reservoir for storing a fluid medium (e.g., saline) within the internal cavity 220B. In particular, the internal cavity 220B of the chamber 202B may be sized to receive and store a predetermined volume of a fluid medium therein, such as that transmitted to the chamber 202B from an external device (e.g., a syringe). By way of example, the predetermined volume of the chamber 202B may range from about 80 milliliters (mL) to about 120 milliliters (mL), and more particularly 100 milliliters (mL).

The fluid reservoir formed by the chamber 202B of the housing 202 may store various fluid mediums therein, such as, for example, saline, an intravenous sugar solution, dextore solutions (D5W), and/or a contrast medium. In other embodiments, the chamber 202B may be configured to receive a fluid reservoir device within the internal cavity 220B, such as a syringe, a bag, and/or the like. In this instance, the fluid reservoir device may be preassembled into the chamber 202B of the housing 202, or alternatively separate from the delivery device 200 such that an operator of the delivery device 200 is required to couple the fluid reservoir device with the housing 202. The housing 202 further includes a proximal wall 205B at the proximal end 204 of the chamber 202B for enclosing the internal cavity 220B. The proximal wall 205B includes a port 207 extending proximally therefrom that is configured and operable to couple the internal cavity 220B of the chamber 202B to a corresponding device, such as, for example, a syringe (not shown). In the present example, the proximal wall 205B includes a plurality of vents and/or holes disposed therethrough to facilitate movement of a floating septum disposed within the chamber 202B (see FIG. 7) without generating a vacuum (i.e. negative pressure) therein.

Still referring to FIG. 6, the chamber 202A of the housing 202 may further include one or more alignment features 203 disposed within the internal cavity 220A. The alignment features 203 may extend from the chamber 202A and into the internal cavity 220A to interface with an exterior surface of the elongated body 256 of the vial assembly 250 to thereby align the vial assembly 250 with the chamber 202A. In this instance, the alignment features 203 comprise an annular array of grooves extending inwardly from the chamber 202A and into the internal cavity 220A. It should be understood that the chamber 202A may include various other suitable alignment features than those shown and described herein without departing from the scope of the present disclosure.

The handle 252 of the vial assembly 250 is integrally secured to the plunger 254 and the plunger 254 extends into the elongated body 256. As will be described in greater detail herein, the plunger 254 is configured to move, and in particular rotate and translate, relative to the elongated body 256 of the vial assembly 250 in response to an actuation of the handle 252. The vial assembly 250 includes a protective shield 253 disposed about at least a portion of the elongated body 256. In the present example, the protective shield 253 extends about a distal segment of the elongated body 256 of the vial assembly 250, however, it should be understood that the protective shield 253 may extend along additional and/or fewer segments of the elongated body 256 without departing from the scope of the present disclosure. Additionally, in some embodiments, the protective shield 253 of the vial assembly 250 may include a plurality of markings and/or indicia disposed along an outer surface thereon. As will be described in greater detail herein, the protective shield 253 is formed of a material configured and operable to inhibit radioactive emissions from a material stored within the elongated body 256 of the vial assembly 250.

Still referring to FIG. 6, the vial assembly further includes a safety tab 259 coupled to the plunger 254, and in particular along an intermediate portion of a longitudinal length of the plunger 254, proximate to the elongated body 256. The safety tab 259 is secured to the plunger 254 and abuts against a proximal end of the elongated body 256. The safety tab 259 is configured to inhibit movement of the plunger 254, and in particular a linear translation of the plunger 254 into the elongated body 256, by engaging the elongated body 256. The safety tab 259 is selectively removable from the vial assembly 250 in response to applying a force against the safety tab 259 opposite of the plunger 254 to thereby extract the safety tab 259 from engagement with the plunger 254 and the elongated body 256. Accordingly, removal of the safety tab 259 provides for a translation of the plunger 254 into the elongated body 256. In other embodiments, a safety lock may comprise a depressible handle interlocked with the handle 252, or alternatively, an electrical switch that removes a physical impediment inhibiting the handle 252 and the plunger 254 from translating relative to the elongated body 256.

Referring now to FIG. 7, the chamber 202B includes a floating septum 221 disposed within the internal cavity 220B with the floating septum 221 movably coupled to an internal tubing line 223 extending between and coupled to the ports 207, 211. Accordingly, the floating septum 221 is translatable within the internal cavity 220B and along the internal tubing line 223. As will be described in greater detail herein, the floating septum 221 is configured to translate within the internal cavity 220B of the chamber 202B, and along the internal tubing line 223, in response to the port 207 receiving a fluid medium therethrough and into the chamber 202B and/or the port 211 releasing a fluid medium therethrough and out of the chamber 202B. The vial assembly 250 is a single-chamber syringe that comprises an internal chamber 251 disposed within the elongated body 256. The vial assembly 250 is configured to selectively deliver a fluid media contained within the elongated body 256, and in particular an internal chamber 251, of the vial assembly 250. In other words, the elongated body 256 is sized to store a fluid media within the internal chamber 251 for delivery to a patient, when the vial assembly 250 is assembled to the delivery device 200, in response to an actuation of the handle 252. In the present example, the fluid media stored within the internal chamber 251 of the elongated body 256 comprises a radioactive material, such as, for example, radioembolizing beads, radioactive microspheres, and the like. As will be described in greater detail herein, the fluid media stored within the internal chamber 251 of the elongated body 256 may be prefilled therein prior to a use of the vial assembly 250 by an operator. The internal chamber 251 may be formed of various materials and/or include various thickness. In the present example, the internal chamber 251 is formed of a plastic and includes a wall thickness of about 9 millimeters (mm).

The vial assembly 250 is formed of a plastic material, such as, for example, polycarbonate, polyethylene, polyethylene terephthalate, or other various plastics. The internal chamber 251 of the vial assembly 250 is encapsulated within a protective shield 253 that is disposed within the elongated body 256 and extends about the internal chamber 251. The protective shield 253 may be formed of a plastic, such as Acrylonitrile Butadiene Styrene (ABS), a lead, tungsten, tin, pewter, and/or other suitable materials for preventing exposure of the radioactive material from within the internal chamber 251. It should be understood that the internal chamber 251 of the vial assembly 250 may be prefilled with a radioactive material prior to an assembly of the vial assembly 250 with the delivery device 200. In this instance, the radioactive material is disposed within the protective shielding 253 of the vial assembly 250 such that radioactive emissions generated by the radioactive material is inhibited by the protective shielding 253 prior to a use of the vial assembly 250 and insertion of the vial assembly 250 into the delivery device 200.

In other embodiments the vial assembly 250 is a dual-chamber syringe and includes at least two internal chambers 251. In this instance, the vial assembly 250 is configured to separately maintain a fluid media within each of the chambers 251 such that the fluid media within the chambers 251 are not exposed to each other and capable of being delivered separately from the vial assembly 250 relative to one another. By way of example only, the vial assembly 250 may be configured and operable in accordance with at least some of the teachings of U.S. App. No. 62/673,628, entitled “Dual Stage Syringe,” filed on even date herewith, the disclosure of which is incorporated by reference herein.

Still referring to FIG. 7, the plunger 254 of the vial assembly 250 extends through the elongated body 256, and in particular, is coupled to the internal chamber 251 of the vial assembly 250 opposite of the handle 252. In particular, the plunger 254 is coupled to the internal chamber 251 such that movement of the plunger 254 generates a pressure within the internal chamber 251 for delivering a material stored therein out of the internal chamber 251. The plunger 254 is a screw-type plunger and includes a threaded portion 257A extending along a longitudinal length of the plunger 254. The threaded portion 257A of the plunger 254 is configured to mesh with a corresponding threaded portion 201 of the vial assembly 250 disposed within the elongated body 256 to facilitate a rotation of the plunger 254 therein. In this instance, a rotation of the handle 252 provides for a simultaneous rotation and linear translation of the plunger 254 through the elongated body 256 and against the internal chamber 251.

In particular, the handle 252 is configured such that an application of a rotatable force thereon (i.e., twisting the handle 252 relative to the elongated body 256) provides a rotation and linear translation of the plunger 254 into the elongated body 256. In this instance, rotating the handle 252 screws the plunger 254 further along the corresponding threaded portion 201 thereby dispensing a material stored within the internal chamber 251 from the delivery device 200 as the plunger 254 applies a continued pressure onto the internal chamber 251. Rotation of the handle 252 provides a slow and controlled rate of fluid disposition from the internal chamber 251 relative to a translation of the handle 252.

Still referring to FIG. 7, the plunger 254 includes a non-threaded portion 257B extending along a longitudinal length of the plunger 254 that is separate from the threaded portion 257A. The non-threaded portion 257B of the plunger 254 is configured to slidably engage one or more mechanisms 212 (e.g., ball bearings) disposed within the elongated body 256 that are configured and operable to facilitate a slidable translation of the plunger 254 therein. In this instance, a linear movement of the handle 252 provides for a simultaneous linear translation of the plunger 254 through the elongated body 256 and against the internal chamber 251. In particular, the handle 252 is configured such that an application of a linear force onto the handle 252 (i.e., pushing the handle 252 relative to the elongated body 256) provides a linear translation of the plunger 254 into the elongated body 256. In this instance, pushing the handle 252 toward the elongated body 256 translates the plunger 254 further into the elongated body 256 and toward the internal chamber 251, thereby dispensing a material stored therein from the delivery device 200 as the plunger 254 applies a continued pressure onto the internal chamber 251. Translation of the handle 252 provides a fast and controlled rate of fluid disposition from the internal chamber 251 relative to a rotation of the handle 252. It should be understood that a translation of the plunger 254 provides for a simultaneous translation of the threaded portion 257A relative to the chamber 202A. With the threaded portion 257A meshed with and coupled to the corresponding threaded portion 201 of the vial assembly 250, the plunger 254 is further configured to translate the threaded portion 201 within the chamber 202A and relative to the internal chamber 251 of the vial assembly 250.

The delivery device 200 further includes a fluid reservoir 216 disposed within the housing 202, and in particular the distal head 208. In the present example, the fluid reservoir 216 may comprise a manifold (e.g. Y-manifold), a connector valve (e.g., a three-way connector and/or T-valve connector), or various other connector mechanisms. In some embodiments, the fluid reservoir 216 may include one or more check valves to prevent a fluid medium flow in certain directions. As will be described in greater detail herein, the fluid reservoir 216 is configured to provide fluidic communication between the vial assembly 250 and the internal cavity 220B of the chamber 202B. Additionally, the fluid reservoir 216 is coupled to the catheter hub 210 such that vial assembly 250 and the internal cavity 220B of the chamber 202B are in fluidic communication with the catheter hub 210.

Still referring to FIG. 7, the fluid reservoir 216 includes a series of delivery lines 214 (i.e., internal tubing) that extends between and fluidly couples the fluid reservoir 216 to the vial assembly 250 and the internal cavity 220B of the chamber 202B, respectively. In particular, at least one of the series of delivery lines 214 is coupled to a port 211 of the internal cavity 220B of the chamber 202B, opposite of the port 207, such that the fluid reservoir 216 is in fluid communication with a fluid medium (e.g., saline) stored within the internal cavity 220B. Further, at least one of the series of delivery lines 214 is coupled to a needle 222 positioned in-line and at a terminal end of the delivery line 214 opposite of the fluid reservoir 216. In this instance, the needle 222 is positioned within the distal head 208 of the housing 202 such that the needle 222 extends into the internal cavity 220A of the chamber 202A.

With the needle 222 extending into the internal cavity 220A of the chamber 202A, it should be understood that the needle 222 is operable to couple with and engage the elongated body 256 when the vial assembly 250 is slidably received therein through the opening 205A. In the present example, the vial assembly 250 includes a septum 255 disposed about a distal end of the elongated body 256, with the septum 255 configured to receive the needle 222 therethrough when the elongated body 256 is received in the internal cavity 220A of the chamber 202A. The septum 255 is formed of an elastomer and is operable to be punctured by the needle 222, thereby facilitating a fluid communication between the internal chamber 251 of the vial assembly 250 and the fluid reservoir 216 via the delivery line 214 coupled to the needle 222. It should be understood that the septum 255 may be formed of various other suitable materials that are configured to securely seal the internal chamber 251 of the vial assembly 250 within the elongated body 256 while being further operable to receive the needle 222 therethrough. Although not shown, it should be understood that the fluid reservoir 216 may be fluidly coupled to the catheter hub 210 via a delivery line 214 coupled thereto and extending therebetween. It should further be understood that in other embodiments the vial assembly 250 may include various other needle connection ports other than the septum 255 shown and described above.

Although not shown, it should be understood that in some embodiments the housing 202 may include an interface surface that has one or more displays (e.g., dosimeter display, sensor output display, viewing window, etc.) to provide an operator of delivery device 200 with real-time feedback of the contents, quantities, and operability of the delivery device 200. Additionally or alternatively, the delivery device 200 may be communicatively coupled to one or more remote displays (e.g., smart device, tablet, etc.). In the embodiments, the delivery device 200 may further include one or more sensors operable to measure a rate of delivery of a fluid media from the delivery device 200, such as, for example, a mixture of a fluid medium contained within the chamber 202B and a radioactive material stored within the internal chamber 251 of the vial assembly 250 (e.g., radioembolizing beads). By way of example only, the one or more sensors (e.g., a dosimeter, a linear encoder, an optical sensor, a linear displacement sensor, a flow sensor, an ultrasonic sensor, a magnetic encoder, a laser distance sensor, an inductance sensor, a radial encoder, a volumetric sensor, mechanical transducers, etc.) may be configured to measure a velocity, pressure, force, displacement, flow, capacitance, radiation, and/or the like of the fluid media delivered from the delivery device 200.

A sensor output display may provide real time monitoring of such measurements calculated by the one or more sensors for an operator's observation during a medical procedure. In particular, such sensors may assist an operator in regulating a delivery after reviewing the measurement outputs from, for example, a display of a device. By way of example, a sensor output display may comprise an LCD screen, a mechanical output, smart device, remote tablet, or other various display outputs positioned along the housing 202 of the delivery device 200 and/or in wireless communication with the delivery device 200.

As briefly described above, the delivery device may include one or more sensors for monitoring radiation levels of the contents of the delivery device 200. By way of example only, such sensors may be highly sensitive radiation sensors (e.g., microcircuit, Geiger counter, etc.) that are configured to detect radiation and measure a total ionizing dose (TID) of radiation. Such sensors may be positioned at various locations within the delivery device 200, and in particular along a travel path of the radioactive materials stored within the delivery device 200 to determine a percent of radioactivity of said materials. A sensor output display may provide real time monitoring of these measurements and comprise various devices, such as, for example, an LCD screen, a mechanical output, smart device, remote tablet, or other various display outputs. It should be understood that in other embodiments the data and information described above may be transmitted (e.g., wirelessly or wired) to a remote device such that a display of the remote device provides said outputs to an operator thereon.

In some embodiments, a viewing window may be positioned along the housing 202, and in particular the chamber 202A where the vial assembly 250 is received therein to provide a visual access to the vial assembly 250. It should be understood that a viewing window may be formed of a radiation shielding material, similar to the protective shield 253 of the vial assembly 250, such that any radioactive material contained within delivery device 200 is sealed therein, thereby minimizing exposure of the radioactive material through a viewing window. By way of example only, the radiation shielding material of a viewing window may be formed of a plastic, such as Acrylonitrile Butadiene Styrene (ABS), a lead glass, or other suitable materials for preventing exposure to radioactive material. Alternatively, a viewing window may comprise a video monitor that is operable to display a visualization within the chamber 202A.

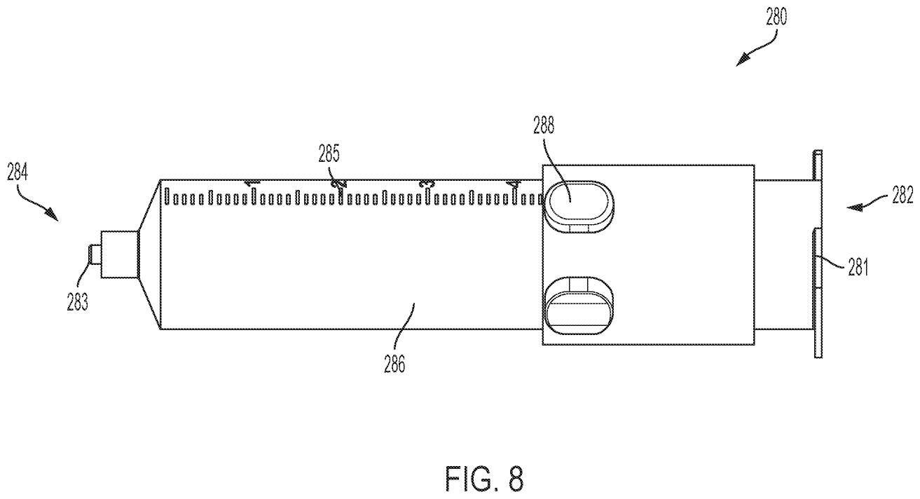

Referring now to FIG. 8, the chamber 202A of the housing 202 may further include a purging syringe 280 stored therein for purposes of flushing the delivery lines 214 and the fluid reservoir 216 of the delivery device 200 prior to loading the vial assembly 250 for use with the delivery device 200. It should be understood that in some embodiments the purging syringe 280 may be preassembled within the internal cavity 220A of the chamber 202A. The purging syringe 280 includes a proximal end 282 and a distal end 284 with an elongated body 286 extending therebetween. The elongated body 286 of the purging syringe 280 is sized and shaped to be received within the chamber 202A of the housing 202, and in particular, to form a press-fit against the internal cavity 220A of the chamber 202A. The elongated body 286 may further include a plurality of indicia and/or markings 285 thereon for purposes of measuring and/or identifying a volume of fluid medium stored therein.

The proximal end 282 includes a collar 281 that is sized and shaped to securely fasten the purging syringe 280 to the chamber 202A. Additionally, the purging syringe 280 includes at least one depressible button 288 extending laterally outward from the elongated body 286, where the depressible button 288 is sized and shaped to be received within the aperture 209 of the housing 202. It should be understood that the depressible button 288 of the purging syringe 280 is configured and operable similar to the depressible button 258 of the vial assembly 250 described above. The distal end 284 of the purging syringe 280 includes a port 283 that is sized and shaped to receive the needle 222 therethrough, thereby establishing a fluid communication between the purging syringe 280 and the fluid reservoir 216 of the delivery device 200 via the needle 222 and the delivery line 214 positioned therebetween.

An exemplary mode of operation of the delivery device 200 is described below. The depiction and accompanying description below is not meant to limit the subject matter described herein or represent an exact description of how a fluid media may be delivered using the delivery device 200, but instead is meant to provide a simple schematic overview to illustrate a general administration of a radioactive media from the delivery device 200 described herein.

Referring to FIGS. 7-8, the delivery lines 214 and the fluid reservoir 216 are initially purged of air using the purging syringe 280. In particular, an external syringe containing a fluid medium (e.g., saline) is coupled to the chamber 202B via a connection at the port 207. The internal cavity 220B of the chamber 202B is filled with the fluid medium from the syringe to a desired volume and, once the chamber 202B is filled, the syringe is decoupled from the port 207. In this instance, the floating septum 221 is translated within the internal cavity 220B as the chamber 202B is filled with the fluid medium via the port 207, thereby causing the septum 221 to translate along the internal tubing line 223 coupled to and extending between the ports 207, 211. In particular, the floating septum 221 is translated distally from the port 207 and proximate to the port 211 as the chamber 202B is filled with the fluid medium. The delivery device 200 is oriented vertically and the purging syringe 280 is pulled back to thereby draw in an amount of fluid medium from the internal cavity 220B of the chamber 202B via the delivery lines 214. In this instance, the purging syringe 280 is pushed forward toward the catheter hub 210 to prime the delivery lines 214 and the fluid reservoir 216 with saline. A delivery line may be coupled to the catheter hub 210 of the delivery device 200, with an opposing end of the delivery line positioned within a collection bowl to receive the flushed medium therein. These steps may be repeated, as necessary, to remove the air from the delivery lines 214 and the fluid reservoir 216 and effectively prime the delivery device 200 for use during a procedure.

With the delivery lines 214 of the delivery device 200 purged of air, the purging syringe 280 is removed from the internal cavity 220A of the chamber 202A via the opening 205A and the vial assembly 250 is inserted therethrough. In particular, the purging syringe 280 is removed in response to depressing the depressible button 288 at the aperture 209 and extracting the elongated body 286 by pulling the collar 281 at the proximal end 282 proximally from the opening 205A. Additionally, the vial assembly 250 is received through the opening 205A and inserted into the housing 202 in response to depressing the depressible button 258 and slidably translating the elongated body 256 into the internal cavity 220A.

Referring back to FIG. 6, the vial assembly 250 is advanced through the chamber 202A, with the chamber 202A continuously applying a predetermined force against the depressible button 258 to thereby maintain the depressible button 258 in a contracted state, until the depressible button 258 is aligned with the aperture 209 of the housing 202. In this instance, a resilient bias of the depressible button 258 extends the depressible button 258 outward from the elongated body 256 due to a termination of the predetermined force thereon. Simultaneous with the receipt of the depressible button 258 in the aperture 209, the septum 255 of the vial assembly 250 contacts the needle 222 within the internal cavity 220A of the chamber 202A. Accordingly, the septum 255 is punctured and the needle 222 is in fluid communication with the internal chamber 251 of the vial assembly 250.

In other words, advancing the vial assembly 250 distally into the chamber 202A provides a series of feedbacks (e.g., visual, audible, tactile, and/or mechanical) to confirm a coupling of the vial assembly 250 with the delivery device 200. In particular, a receipt of the depressible button 258 in the aperture 209 may provide a visual, audible, tactile and mechanical feedback to an operator that the vial assembly 250 is coupled to the delivery device 200. Additionally, a puncture of the septum 255 by the needle 222 may provide an audible, tactile and mechanical feedback to an operator that the vial assembly 250 is in fluid communication with the delivery device 200. In this instance, with the internal chamber 251 now in fluidic communication with the delivery device 200, any advancement of the handle 252 provides for the delivery of the radioembolizing beads stored within the internal chamber 251 of the vial assembly 250.

Referring to FIG. 7, the catheter hub 210 of the delivery device 200 may be coupled to a catheter (e.g., microcatheter) via a delivery line extending therebetween. It should be understood that in other embodiments the catheter hub 210 may be coupled to a catheter prior to assembling the vial assembly 250 into the chamber 202A of the delivery device 200. With the vial assembly 250 storing radioembolizing beads within the internal chamber 251 with the protective shield 253 disposed thereover, an operator is not required to manipulate any vials containing radioactive material during the medical procedure. Rather, once the vial assembly 250 is assembled into the housing 202 of the delivery device 200 an operator is not required to directly handle the radioembolizing beads any further, thereby reducing the risk of radiological or biological contamination by human error during the procedure.

With the internal cavity 220B of the chamber 202B loaded with a fluid media and the vial assembly 250 fully assembled into the delivery device 200, an operator may selectively actuate the delivery device 200 to deliver a controlled mixture of the therapeutic particles (e.g., radioembolizing beads) from the vial assembly 250 and fluid media from the chamber 202B during a procedure. As briefly noted above, the delivery device 200 may be communicatively coupled to a remote device, such as, for example, a tablet, a computer, a mobile device, and/or the like. The remote device may receive and display delivery information along an interface display of the remote device for an operator of the delivery device 200 to monitor as the delivery device 200 is in use during a procedure. For example, the delivery information displayed along the remote device may include, but is not limited to, a rate of flow (ml/min), a current volume of media in the chambers 202A, 202B, an infused volume of media from the chambers 202A, 202B, a remaining percentage of radioactive activity stored within the delivery device 200, and/or the like.

Referring back to FIG. 5, to administer a dose of radioactive material with the delivery device 200, the handle 252 of the vial assembly 250 is actuated to translate the plunger 254 proximally away from the elongated body 256. In this instance, a negative pressure is generated between the internal chamber 251 of the vial assembly 250 and the internal cavity 220B of the chamber 202B, which are in communication with one another through the air-purged delivery lines 214. Accordingly, pulling the plunger 254 proximally extracts a fluid media stored within the internal cavity 220B (e.g., saline) through the delivery lines 214 and into the internal chamber 251 of the vial assembly 250 via the needle 222. In particular, the floating septum 221 is translated proximately toward the port 207 and distally away from the port 211 as the chamber 202B is emptied of the fluid medium. This suction of fluid media into the internal chamber 251, where the therapeutic particles are stored, causes a mixture of the two substances therein to form a suspension fluid.

Once the handle 252 and the plunger 254 are pulled proximally to a fullest extent, the handle 252 may be actuated to translate the plunger 254 distally toward the elongated body 256 to generate a positive pressure. The handle 252 may be actuated by either rotating the handle 252 to deliver a slow, controlled dose of the radioactive mixture or by translating the handle 252 to deliver a fast, controlled dose. In some embodiments, depression of the depressible button 258 toward the elongated body 256 may be required to translate the handle 252 and the plunger 254 to deliver a fast, controlled dose of the mixture. In this instance, the depressible button 258 may serve as a secondary safety mechanism for the delivery device 200 when administering a fast dose of the mixture.

Referring back to FIG. 7, a dose of the mixture formed in the internal chamber 251 of the vial assembly 250 is effectively transferred through the needle 222 and into the fluid reservoir 216, where the dose is thereby delivered out from the delivery device 200 through the catheter hub 210. With the delivery device 200 coupled to a catheter via the catheter hub 210, the mixture may be delivered to a patient intravenously by positioning the catheter at a target treatment site within a patient. Additional doses may be delivered by the delivery device 200 by repeating the actuation of the handle 252 described above to refill and purge the mixture of fluid mediums until either a sufficient dose has been administered to a patient (e.g., a sensor output reading from a dosimeter sensor drops to a predetermined level), the internal chamber 251 is depleted, and/or or stasis is achieved. Sensor output displays may provide an operator with real-time informational feedback of the force, pressure, and/or flow of the mixture delivered from the delivery device 200 to the catheter via one or more sensors contained within the delivery device 200. By monitoring sensor output display an operator is able to regulate the delivery of the radioembolizing beads to the patient and cease delivery when desired.

In instances where a fluid media stored within the internal cavity 220B of the chamber 202B is depleted prior to a completion of the procedure, additional fluid media (e.g., saline) may be refilled into the chamber 202B during the procedure via the port 207. At a conclusion of the procedure, the delivery device 200 may be discarded. In some embodiments the delivery device 200 may include a transducer therein such that an operator may be capable of actuating the delivery device 200 from a remote location such that an operator is located distally from the radioactive material contained within the delivery device 200.

Although not shown, it should be understood that the delivery device 200 may further include a device stand that is sized and shaped to removably receive the delivery device 200 thereon. The delivery device may be configured and operable to temporarily maintain the delivery device 200 during a medical procedure. Accordingly, the device stand may facilitate and preserve a sterilization of the delivery device 200 prior to, during, and after use of the delivery device 200 for a procedure.

### III. Semi-Automatic Handheld Delivery Device

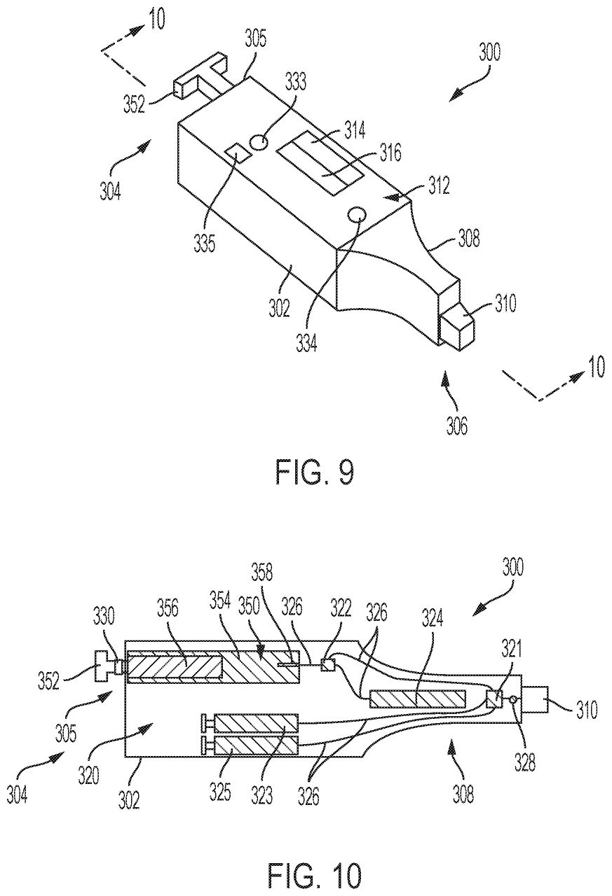

FIGS. 9-10 show another embodiment of a delivery device 300 that is configured and operable to deliver a radioactive material (e.g., radioembolizing beads) while reducing radioactive emissions during use of the delivery device 300. It should be understood that the delivery device 300 of the present example may be configured and operable similar to the delivery device 200 described above, as the delivery device 300 is substantially similar to the delivery device 200 except for the differences explicitly noted herein.

Referring specifically to FIG. 9, the delivery device 300 includes a housing 302 extending between a proximal end 304 and a distal end 306, with the distal end 306 of the housing 302 including an elongated housing 308 extending distally therefrom. The elongated housing 308 of the delivery device 300 includes a distal tip 310 that comprises a catheter hub for coupling the delivery device 300 to an external device, such as, for example, a catheter. The housing 302 of the delivery device 300 defines an internal cavity 320 disposed therein (See FIG. 10). As will be described in greater detail herein, the internal cavity 320 defined by the housing 302 stores one or more devices (e.g., syringes, fluid reservoirs, valves, and the like) within the delivery device 300. The housing 302 further includes an interface surface 312 positioned between the proximal end 304 and the distal end 306 of the delivery device 300, with the interface surface 312 including one or more switches for actuating the one or more devices stored within and coupled to the delivery device 300. The interface surface 312 further includes one or more displays to providing feedback (e.g., visual) of an output and/or operability of the one or more devices stored within the delivery device 300.

In the present example, the interface surface 312 of the delivery device 300 includes at least a dosimeter display 314, a sensor output display 316, a contrast switch 333, a flush switch 334, and a saline switch 335. It should be understood that a position of the displays 314, 316 and switches 333, 334, 335 shown and described herein are merely for illustrative purposes only such that a location of the displays 314, 316 and the switches 333, 334, 335 may vary without departing from the scope of the present disclosure. As will be described in greater detail below, each switch 333, 335 is communicatively coupled to and configured to actuate a respective device (e.g., a contrast syringe 323, a saline syringe 325, respectively) contained within the internal cavity 320 of the housing 302. Accordingly, manipulating the switches 333, 335 along the interface surface 312 of the housing 302 may provide for an automatic delivery of a fluid medium contained within the syringes 323, 325, respectively.

Referring now to FIG. 10, the internal cavity 320 of the housing 302 includes at least a pair of connector valves 321, 322, a contrast syringe 323, a fluid reservoir 324, a saline syringe 325, and a syringe 350. The various devices disposed within the internal cavity 320 of the housing 302 are fluidly coupled to one another via a series of delivery lines 326 disposed within the internal cavity 320 of the housing 302 and extending therebetween. In particular, the syringe 350 is fluidly coupled to the first connector valve 322 via a delivery line 326 extending therebetween. The syringe 350 includes an external chamber 354, an internal chamber 356 disposed within the external chamber 354, and an internal needle 358 disposed within the external chamber 354. The internal chamber 356 is sized and shaped to be received within the external chamber 354. In other words, the syringe 350 is a dual-chamber syringe that is capable of storing multiple fluid mediums therein, such that a fluid medium stored within each of the respective chambers 354, 356 are separated from one another. In the present example, a fluid medium stored in the external chamber 354 of the syringe 350 comprises a saline media and a fluid medium stored in the internal chamber 356 of the syringe comprises a radioactive media, such as, for example, radioembolizing beads. It should be understood that in other embodiments the pair of connector valves 321, 322 may be comprise various other devices, such as, for example, a manifold.

The syringe 350 further includes a handle 352 coupled to the internal chamber 356 such that the internal chamber 356 is movable within the internal cavity 320, and in particular within the external chamber 354, in response to an actuation (e.g., linear translation) of the handle 352 relative to the housing 302 of the delivery device 300. It should be understood that the external chamber 354 of the syringe 350 is fixedly secured within the internal cavity 320 of the housing 302 such that the external chamber 354 is immovable in response to an actuation of the handle 352. The handle 352 extends proximally outward from the housing 302 at the proximal end 304 such that the handle 352 of the syringe 350 is accessible by an operator of the delivery device 300 despite the syringe 350 being disposed within the internal cavity 320 of the housing 302. The handle 352 of the syringe 350 extends distally from the internal cavity 320 via a syringe opening 305 located at the proximal end 304 of the housing 302.

It should be understood that with the internal needle 358 of the syringe 350 is fixedly secured within the external chamber 354 along an end opposite of the internal chamber 356 of the syringe 350. With the internal chamber 356 movably coupled to the handle 352 within the external chamber 354 and the internal needle 358 fixedly disposed within the external chamber 354, a translation of the handle 352 may provide an interaction of the internal chamber 356 and the internal needle 358. More specifically, and as will be described in greater detail herein, an actuation of the handle 352 (e.g., translating the handle 352 distally toward the distal end 306 of the delivery device 300) generates a positive pressure in the external chamber 354 of the syringe 350 as the internal chamber 356 moves within the external chamber 354.

Still referring to FIG. 10, upon an initial actuation of the handle 352, a fluid media stored within the external chamber 354 (e.g., saline) is effectively transmitted from the external chamber 354 to the first connector valve 322 via the delivery line 326 coupled to and disposed therebetween. In this instance, a continued translation of the handle 352 in toward the distal end 306 of the delivery device 300 causes the internal chamber 356 to encounter the internal needle 358 within the external chamber 354. In this instance, the handle 352 is operable to establish fluid communication between the internal chamber 356 and the external chamber 354 in response to the internal needle 358 puncturing the internal chamber 356. Accordingly, a fluid media stored within the internal chamber 356 (e.g., radioembolizing beads) is thereby transferable to the external chamber 354, and with the external chamber 354 depleted of a fluid medium in response to the initial actuation of the handle 352, the radioembolizing beads stored in the internal chamber 356 may be effectively delivered to the first connector valve 322 via the delivery line 326 coupled therebetween.

The first connector valve 322 disposed within the internal cavity 320 of the housing 302 is similar to the fluid reservoir 216 of the delivery device 200 described above, such that the first connector valve 322 may comprise a Y-manifold, a three-way check valve assembly, and/or the like. The first connector valve 322 provides fluidic communication between the syringe 350 and the fluid reservoir 324 via the series of delivery line 326. Further, the first connector valve 322 is in fluidic communication with a second connector valve 321, which is positioned adjacent to the distal end 306 of the housing 302 an disposed within the elongated housing 308. The contrast syringe 323 and the saline syringe 325 are fluidly coupled to the second connector valve 321.

Still referring to FIG. 10, the contrast syringe 323 is configured to store a fluid medium therein, and in the present example the contrast syringe 323 includes a contrast medium stored therein. The saline syringe 325 is similarly configured to store a fluid medium therein, and in the present example the saline syringe 325 includes a saline medium stored therein. It should be understood that syringes 323, 325 may include various other suitable fluid media, and in some instances may include identical substances stored therein. Although not shown, it should also be understood that additional or fewer syringes 323, 325, 350 may be included within the internal cavity 320 of the delivery device 300. Further, although the syringes 323, 325 are shown as having a size and shape that are different than the syringe 350, it should be understood that the syringes 323, 325, 350 may comprise various suitable shapes and sizes that may be stored within the internal cavity 320 of the housing 302 without departing from the scope of the present disclosure. Additionally, it should further be understood that a position of the syringes 323, 325, 350 shown and described herein are merely for illustrative purposes such that a location of the syringes 323, 325, 350 may vary without departing from the scope of the present disclosure.

The contrast syringe 323 is in fluidic communication with the second connector valve 321 via the delivery line 326 and the saline syringe 325 is in fluidic communication with the second connector valve 321 via a separate delivery line 326. In this instance, the fluid media contained within the syringes 323, 325, 350, respectively, are separated and isolated from one another within the internal cavity 320 of the housing 302 until arriving at the second connector valve 321. In other words, the second connector valve 321 serves as an integration site for the fluid media contained within the syringes 323, 325, 350. It should be understood that in some embodiments the syringes 323, 325, 350 may be removably received within the internal cavity 320, and in particular, may not be preassembled within the delivery device 300. Accordingly, an operator is able to determine which syringes 323, 325, 350 to load into the delivery device 300 based on a particular medical procedure to be performed with the delivery device 300. Alternatively, in some instances the syringes 323, 325, 350 may be preloaded into the delivery device 300 such that an operator is not required to insert one or more of the syringes 323, 325, 350 into the internal cavity 320 during a medical procedure.

Still referring to FIG. 10, in the present example the switches 333, 334, 335 are configured to be electrically actuated to thereby actuate the syringes 323, 325, 350, respectively. In other versions, one or more of the switches may be configured to be hydraulically, mechanically, and/or pneumatically actuated to actuate the syringes 323, 325, 350. In particular, the contrast switch 333 is configured and operable to actuate the contrast syringe 323, such that actuation of the contrast switch 333 administers a transmission of a fluid medium stored within the contrast syringe 323 (e.g., contrast medium) through the series of delivery lines 326 and into the second connector valve 321. The saline switch 335 is configured and operable to actuate the saline syringe 325, such that actuation of the saline switch 335 administers a transmission of a fluid medium stored within the saline syringe 325 (e.g., saline medium) through the series of delivery lines 326 and into the second connector valve 321. The flush switch 334 serves as a safety lock and is configured to permit delivery of a first fluid medium (e.g., saline) from the external chamber 354 of the syringe 350. Accordingly, actuation of the handle 352 of the syringe 350 is inoperable to deliver a first fluid medium stored within the external chamber 354 until the flush switch 334 is actuated.

Referring back to FIG. 9, it should be understood that an actuation of the switches 333, 334, 335 may comprise interacting with the interface surface 312 along the housing 302 of the delivery device 300 by contacting the switches 333, 334, 335 (i.e., one click actuation), by continuously engaging the switches 333, 334, 335, and the like. It should further be understood that in other versions the switches 333, 334, 335 may be remotely located relative to the housing 302 such that delivery device 300 may be actuated wirelessly via a remote device. In either instance, actuation of the switches 333, 335 of the delivery device 300 provides an automatic transmission of the respective fluid media contained therein. It should be understood that a pressure, flow, and/or fill rate employed by the syringes 323, 325 in response to an actuation of the switches 333, 335 may be preprogrammed such that actuation of the switches 333, 335 provides autonomous transmission of the fluid medium at the predetermined rate.

For example, a desired pressure, flow, and/or fill rate of the delivery device 300 may be selectively inputted at the interface surface 312 and/or via a remote device communicatively coupled to the delivery device 300 prior to commencing a procedure with the delivery device 300. However, it should be understood that a delivery of a fluid media stored within the syringe 350, and in particular an internal chamber 356 of the syringe 350 (e.g., radioembolizing beads) remains fully manual via the handle 352. Accordingly, an effective flow and pressure rate for delivering the one or more mediums of the syringe 350 stored within the chambers 354, 356 is mechanically determined based on an application of force onto the handle 352.

Referring back to FIG. 10, the delivery device 300 may include one or more sensors disposed within the internal cavity 320 of the housing 302. As described in greater detail above with respect to the delivery device 200, the one or more sensors (e.g., a dosimeter, a linear encoder, an optical sensor, a linear displacement sensor, a flow sensor, an ultrasonic sensor, a magnetic encoder, a laser distance sensor, an inductance sensor, a radial encoder, a volumetric sensor, mechanical transducers, etc.) may be configured and operable to measure a flow, capacitance, radiation, volume, and/or the like of the various fluid mediums stored within and administered by the delivery device 300. In the present example, the delivery device 300 includes a dosimeter sensor 328 and a displacement sensor 330. In particular, the dosimeter sensor 328 is disposed within the housing 302, and in particular within the elongated housing 308 of the delivery device 300. The dosimeter sensor 328 is fluidly coupled to the second connector valve 321 via the delivery line 326 extending therebetween and is operable to measure a radiation level of the fluid media administered through the dosimeter sensor 328 from the second connector valve 321 to the catheter hub at the distal tip 310 of the delivery device 300. The dosimeter sensor 328 is communicatively coupled to the dosimeter display 314 positioned along the interface surface 312 such that an operator of the delivery device 300 may monitor data detected by the dosimeter sensor 328 thereon.

The displacement sensor 330 is positioned along the handle 352 of the syringe 350 such that the displacement sensor 330 is positioned external from the internal cavity 320 of the housing 302. The displacement sensor 330 is operable to measure a linear displacement of the handle 352 relative to the housing 302 to determine a force, pressure, and/or flow of the fluid medium administered from the syringe 350 to the catheter hub at the distal tip 310. The displacement sensor 330 is communicatively coupled to the sensor output display 316 positioned along the interface surface 312 such that an operator of the delivery device 300 may monitor data detected by the displacement sensor 330 thereon. It should be understood that additional and/or fewer sensors, displays, switches, and/or syringes, may be provided in the delivery device 300 without departing from the scope of the present disclosure.

Still referring to FIG. 10, in an exemplary mode of operation of the delivery device 300, an operator may use the delivery device 300 in a manner substantially similar to that of the delivery device 200 described above. For instance, with syringes 323, 325, 350 assembled in the internal cavity 320 of the housing 302, the delivery device 300 is coupled to an external catheter via the catheter hub positioned at the distal tip 310. In this instance, the flush switch 334 is actuated by depressing the flush switch 334 downward, thereby unlocking a movement of the handle 352 of the syringe 350. Accordingly, an operator may apply a predetermined force onto the handle 352, and more particularly push the handle 352 distally toward the housing 302 to commence a flush of the syringe 350, the connector valves 321, 322, and the catheter hub at the distal tip 310 via the series of delivery lines 326 disposed therebetween. The delivery device 300 is flushed with the fluid medium stored within the external chamber 354 of the syringe 350 in response to actuating the handle 352, where the fluid medium stored within the external chamber 354 may comprise, for example, a saline medium. Accordingly, the saline is transferred through the catheter hub at the distal tip 310 and into an external catheter coupled to the delivery device 300 thereto, thereby purging the corresponding catheter system of any air contained therein.

Continuing a distal translation of the handle 352 distally into the housing 302, while the flush switch 334 remains continuously depressed along the interface surface 312, provides a first feedback to an operator of the delivery device 300. By way of example only, the delivery device 300 may be configured to generate a feedback (e.g., visual, audio, tactile, mechanical, etc.) in response to a depletion of a fluid medium (e.g., saline) stored within the external chamber 354 of the syringe 350. Upon a depletion of the external chamber 354, an operator actuates one or more of the switches 333, 335 to transmit a fluid medium stored therein, respectively. Continued translation of the handle 352 distally into the housing 302 of the delivery device 300 generates a second feedback in response to the internal needle 358 puncturing the internal chamber 356 of the syringe 350. In this instance, fluidic communication between the internal chamber 356 and the external chamber 354 is formed such that a fluid media stored within the internal chamber 356 (e.g., radioembolizing beads) may be effectively transferred therefrom.

Still referring to FIG. 10, in this instance the handle 352 is manually retracted in a proximal direction relative to the housing 302, which requires continued actuation of the flush switch 334 along the interface surface 312. A negative pressure is generated within the syringe 350 in response such that the negative pressure causes the fluid media stored within the fluid reservoir 324 to transfer through the first connector valve 322 and into the internal chamber 356 via the external chamber 354. Accordingly, a fluid medium stored within the fluid reservoir 324 is intermixed with the fluid media contained within the internal chamber 356. Subsequent distal translation of the handle 352 toward the housing 302 transfers the mixture of fluid mediums from the syringe 350, through the first connector valve 322, and into the second connector valve 321. In this instance, an operator may further actuate either switch 333, 335 to thereby transfer a contrast medium and/or saline medium from the contrast syringe 323 and/or saline syringe 325, respectively, to the second connector valve 321.

Accordingly, a further mixture of mediums is formed at the second connector valve 321 from the one or more fluid media contained within the syringes 323, 325, 350. Thus, prior to the mixture of fluid media being delivered through a catheter hub at the distal tip 310 of the delivery device 300 and into an external catheter coupled thereto, the delivery device 300 is operable to mix multiple fluid mediums therein for delivery to a patient. The sensor output display 316 along the interface surface 312 provides real time informational feedback of the force, pressure, and/or flow of the mixture delivered from delivery device 300 to the catheter via the displacement sensor 330. The displacement sensor 330 allows an operator to regulate a delivery of the radioembolizing beads to the patient and cease delivery when desired. An operator may continue delivering the radioembolizing beads from the delivery device 300 until the dosimeter display 314 indicates that a radiation exposure level measured by the dosimeter sensor 328 has dropped to an acceptable level (e.g., approximately zero radioactive material remaining in delivery device 300).

### IV. Automatic Handheld Delivery Device

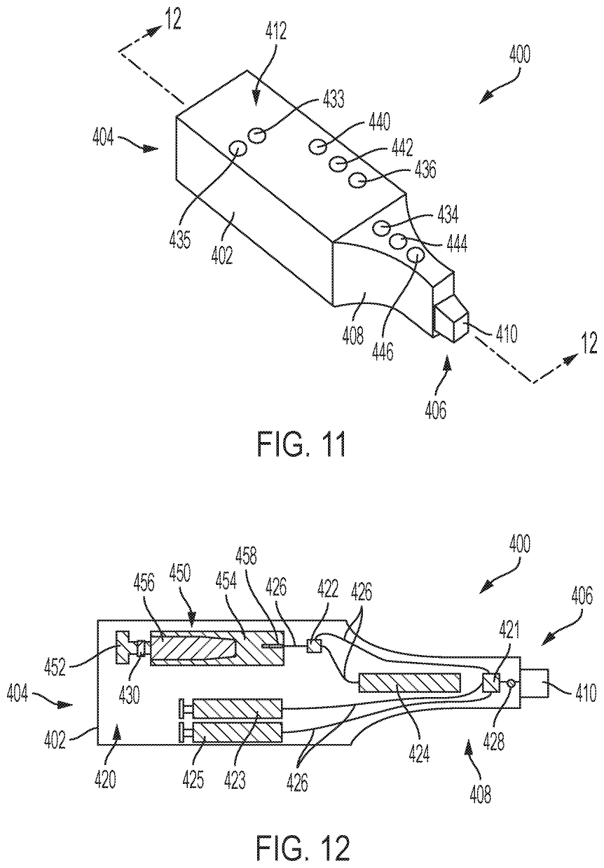

FIGS. 11-12 shows another embodiment of a delivery device 400 that is configured and operable to deliver a radioactive material (e.g., radioembolizing beads) while reducing radioactive emissions during use of the delivery device 400. It should be understood that the delivery device 400 of the present example may be configured and operable similar to the delivery device 200, 300 described above as the delivery device 400 is substantially similar to the delivery device 200, 300 except for the differences explicitly noted herein.

Referring specifically to FIG. 11, the delivery device 400 includes a housing 402 extending between a proximal end 404 and a distal end 406, with the distal end 406 of the housing 402 including an elongated housing 408 extending distally therefrom. The elongated housing 408 of the delivery device 400 includes a catheter hub 410 for coupling the delivery device 400 to an external device, such as, for example, a catheter. The housing 402 of the delivery device 400 defines an internal cavity 420 disposed therein (See FIG. 12). Similar to the delivery device 300 described above, the internal cavity 420 defined by the housing 402 of the delivery device 400 stores one or more devices (e.g., syringes, fluid reservoirs, valves, manifolds, and the like) within the delivery device 400, such as the pair of connector valves 421, 422, the contrast syringe 423, the manifold and/or fluid reservoir 424, the saline syringe 425, and the syringe 450. It should be understood that the connector valves 421, 422, the syringes 423, 425, 450 and the fluid reservoir 424 of the delivery device 400 are configured and operable substantially similar to those described above with respect to the delivery device 300. In some embodiments, the pair of connector valves 421, 422 may comprise various other devices, such as, for example, a manifold.

Still referring to FIG. 11, the housing 402 further includes an interface surface 412 positioned between the proximal end 404 and the distal end 406 of the delivery device 400, with the interface surface 412 including one or more switches for actuating the one or more devices stored within and coupled to the delivery device 400. The interface surface 412 further includes one or more displays for providing feedback (e.g., visual) of an output and/or operability of the one or more devices stored within the delivery device 400. The delivery device 400 includes a contrast switch 433, a flush switch 434, and a saline switch 435 positioned along the interface surface 412 that are substantially similar to the switches 333, 334, 335 of the delivery device 300 described above. Unlike the delivery device 300, however, the delivery device 400 does not include a dosimeter display or sensor output display along the interface surface 412.

Rather, the delivery device 400 includes a first engagement switch 440 and a first dispense switch 442 positioned along the interface surface 412. Further, the elongated housing 408 includes a second engagement switch 444 and a second dispense switch 446 positioned proximate to the switches 440, 442. Although not shown, it should be understood that the switches 444, 446 may alternatively be located along the interface surface 412. It should further be understood that a location of the switches along the interface surface 412 of the delivery device 400 are merely for illustrative purposes such that the switches may be positioned along various other surfaces of the delivery device 400 without departing from the scope of the present disclosure.

Still referring to FIG. 11, the delivery device 400 is configured to deliver a fluid medium stored within the respective syringes 423, 425, 450 in response to an actuation (e.g., depression) of a corresponding switch 433, 434, 435. In other words, as will be described in greater detail below, dissimilar to the flush switch 334 of the delivery device 300 described above, actuation of the flush switch 434 of the delivery device 400 provides for an automated translation of the handle 452 of the syringe 450. In the present example, the switches 433, 434, 435 are configured to be electrically actuated to flush the syringes 423, 425, 450, respectively. In other versions, the switches 433, 434, 435 may be configured to be hydraulically, mechanically, and/or pneumatically actuated to flush the syringes 423, 425, 450.

Referring to FIG. 12, the handle 452 of the syringe 450 is disposed within the housing 402 of the delivery device 400 such that the delivery device 400 does not include a syringe opening at the proximal end 404 of the housing 402. Accordingly, actuation of the handle 452 is controlled at least in part by the flush switch 434 along the interface surface 412, rather than by a manual actuation as the handle 352 of the delivery device 300 requires as described in greater detail above.

In an exemplary mode of operation, the delivery device 400 is employed in a substantially similar manner as the delivery device 300 described above. For instance, with the syringes 423, 425, 450 assembled in the internal cavity 420 of the housing 402, the delivery device 400 is coupled to a catheter via the catheter hub 410 of the housing 402. With the catheter positioned within a target treatment site of a patient's body, the flush switch 434 is actuated to automatically translate the handle 452 distally to thereby flush a fluid medium stored within the external chamber 454 of the syringe (e.g., saline) therefrom and into the connector valves 421, 422 and the catheter hub 410, respectively, via the series of delivery lines 426. Accordingly, the saline is transferred through the catheter hub 410 and into the catheter coupled thereto thereby purging the catheter system of any air contained therein.

Referring back to FIG. 11, continued actuation of the flush switch 434 provides a continued translation of the handle 452 in a distal direction until a first feedback (e.g., visual, audio, tactile, mechanical, etc.) is generated. The first feedback may be indicative of a depletion of the fluid medium stored within the external chamber 454 (e.g., saline) of the syringe 450. In this instance, an operator ceases actuating (e.g., pressing) the flush switch 434 and may actuate either the contrast switch 433 and/or the saline switch 435 to thereby transmit a contrast medium or saline medium to the second connector valve 421, respectively, from either of the syringes 423, 425.

Actuating the first and second engagement switches 440, 444 concurrently provides for a translation of the internal needle 458 within the external chamber 454 of the syringe 450 and toward the internal chamber 456. Accordingly, dissimilar to the delivery device 300 described above, the internal needle 458 of the delivery device 400 is movable within the external chamber 454 in response to an actuation of the engagement switches 440, 444. The internal needle 458 is translated within the external chamber 454 until the internal needle 458 encounters the internal chamber 456 within the external chamber 454. In this instance, the internal chamber 456 is punctured by the internal needle 458 thereby establishing access to a fluid media stored within the internal chamber 456 (e.g., radioembolizing beads). A second feedback (e.g., visual, audio, tactile, mechanical, etc.) is generated indicating fluidic communication to the internal chamber 456 being established.

Referring back to FIG. 12, manual retraction of the handle 452 in a proximal direction toward the proximal end 404 of the delivery device 400 is provided in response to actuating the fill switch 436. Proximal retraction of the handle 452 generates a negative pressure within the syringe 450 thereby causing a fluid medium stored within the fluid reservoir 424 to be drawn into the internal chamber 456 via the series of delivery lines 426 and the first connector valve 422 coupled therebetween. With the internal chamber 456 having received a fluid medium of the fluid reservoir 424 therein, a mixture of the mediums is formed within the internal chamber 456. An operator may cease actuating (e.g., pushing) the fill switch 436 to thereby terminate a proximal translation of the handle 452.

In this instance, with a mixture of fluid mediums from the fluid reservoir 424 and the internal chamber 456 formed within the internal chamber 456, actuating both the dispense switches 442, 446 provides for a translation of the handle 452 in a distal direction toward the distal end 406 of the delivery device 400, thereby generating a positive pressure to deliver the mixture from the syringe 450, through the first connector valve 422, and into the second connector valve 421. In this instance, either of the switches 433, 435 may be actuated to thereby transfer a contrast agent and/or saline from the contrast syringe 423 and/or saline syringe 425, respectively. Accordingly, an additional mixture may be formed at the second connector valve 421 with the fluid media transferred from the syringes 423, 425, 450 prior to the mixture being delivered through the catheter hub 410 and into a connecting catheter thereon. As described in greater detail above with respect the delivery devices 100, 200, 300, the delivery device 400 of the present example may include one or more sensors (e.g., a dosimeter, a linear encoder, an optical sensor, a linear displacement sensor, a flow sensor, an ultrasonic sensor, a magnetic encoder, a laser distance sensor, an inductance sensor, a radial encoder, a volumetric sensor, mechanical transducers, etc.) therein for detecting, measuring, and outputting data relating to the therapeutic particles administered by the delivery device 400 to a patient.

V. Mechanical Delivery Device with Removable Sled Assembly

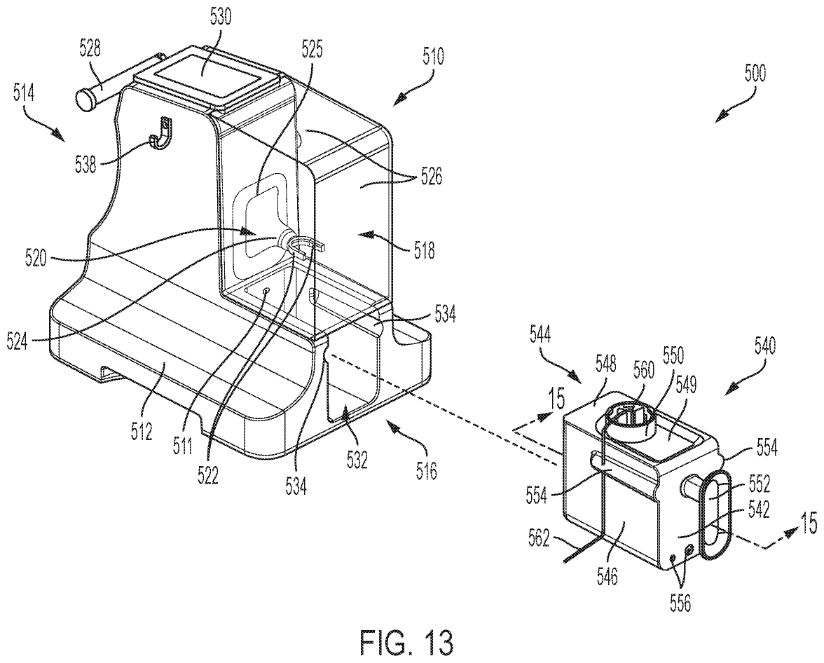

FIGS. 13-29 show another embodiment of a delivery device 500 that is configured and operable to deliver a radioactive material (e.g., radioembolizing beads) while reducing radioactive emissions during use of the delivery device 500. It should be understood that the delivery device 500 of the present example may be configured and operable similar to the delivery device 100 described above as the delivery device 500 is substantially similar to the delivery device 100 except for the differences explicitly noted herein.

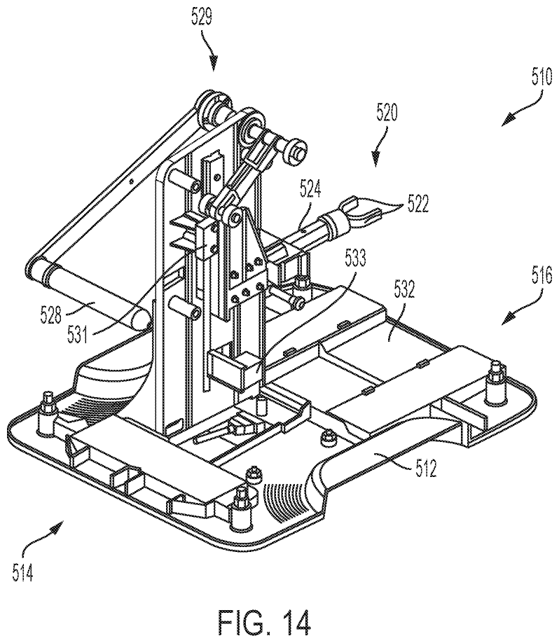

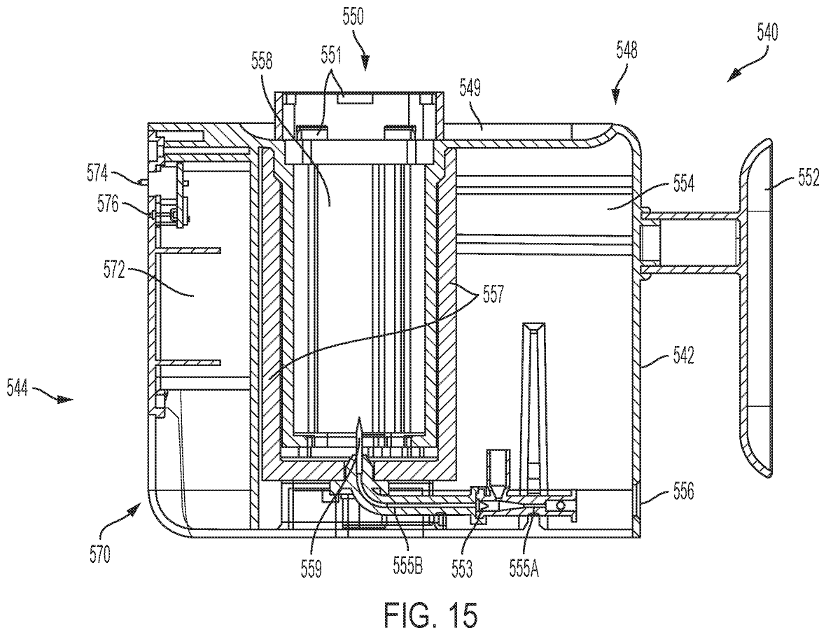

Referring initially to FIG. 13, the delivery device 500 comprises a console assembly 510 and a sled assembly 540 that are operable to transition between a coupled state and decoupled state relative to one another. The console assembly 510 of the delivery device 500 comprises a base 512 defined by and extending between a proximal end 514 and a distal end 516. The proximal end 514 of the base 512 includes a handle (delivery handle) 528 movably coupled to the console assembly 510 and an interface display 530 positioned on the console assembly 510. As will be described in greater detail herein, the interface display 530 is operable to transmit information and/or data to an operator of the delivery device 500, and in particular data detected by an electrical system of the delivery device 500 which may comprise one or more sensors disposed within the delivery device 500 (See FIG. 14). It should be understood that the delivery device 500 may include an electrical microprocessor that operates the interface display 530. In other embodiments, the interface display 530 may comprise a remote smart device, a tablet, and/or the like.

The proximal end 514 of the base 512 further includes an attachment device 538 that is configured to securely retain an external device to the base 512 of the console assembly 510. The attachment device 538 is operable to facilitate an attachment of a complimentary device to the console assembly 510 for use with the delivery device 500 during a procedure. In the present example, the attachment device 538 is a hook assembly extending outwardly from a side of the base 512 that is sized and shaped to attach a saline bag (i.e., the complimentary device) to the console assembly 510. In other embodiments, the engagement mechanism may comprise various other forms or configurations for securing a complimentary device to the console assembly 510.

Still referring to FIG. 13, the distal end 516 of the console assembly 510 defines a vial containment region 518 that is sized and shaped to receive the console assembly 510 therein, as will be described in greater detail herein. The console assembly 510 further includes a vial engagement mechanism 520 extending from the base 512 adjacent to the distal end 516. In particular, the vial engagement mechanism 520 extends laterally outward from the base 512 of the console assembly 510 toward the distal end 516. The vial engagement mechanism 520 is positioned within the vial containment region 518 of the console assembly 510 and is movably coupled to the handle 528. In particular, the handle 528 of the console assembly 510 is operable to move, and in particular translate, the vial engagement mechanism 520 within the vial containment region 518 in response to an actuation of the handle 528. It should be understood that an ergonomic design of the handle 528 serves to facilitate delivery of a dose from the delivery device 500 through a range of various operator angles relative to the base 512 to thereby enhance a mobility of performing a procedure with the delivery device 500.

Referring now to FIG. 14, the console assembly 510 includes a mechanical assembly 529 disposed within the base 512 that is configured and operable to convert a manual motion of the handle 528 to a corresponding linear displacement of the vial engagement mechanism 520. In the present example, the mechanical assembly 529 is coupled to the handle 528 and the vial engagement mechanism 520 such that selective actuation of the handle 528 at the proximal end 514 causes a simultaneous actuation of the vial engagement mechanism 520 at the distal end 516. As will be described in greater detail herein, the mechanical assembly 529 of the present example allows for fluid volume control and fluid flow volume control during a dose delivery with the delivery device 500. It should be understood that a mechanical configuration of the mechanical assembly 529 of the present example may comprise various linkages, gears, pullies, springs and/or the like that are specifically configured to amplify a force applied to the handle 528 with a corresponding displacement of the vial engagement mechanism 520. In some embodiments, the mechanical assembly 529 may comprise and/or be substituted by one or more electrically-driven systems, motors, and/or other devices operable to provide for a movement of the vial engagement mechanism 520 relative to the vial containment region 518 and/or provide a feedback to an operator as the handle 528 is actuated.

In other embodiments the mechanical assembly 529 may be configured such that the handle 528 may be actuated (i.e., moved) in various other arrangements or orientations than that shown and described herein to generate a corresponding linear displacement of the vial engagement mechanism 520. For example, the mechanical assembly 529 of the console assembly 510 may be configured to convert a linear, rotational, lateral and/or other various motions of the handle 528 to generate a disproportionate displacement of the vial engagement mechanism 520, with the displacement exceeding a force applied at the handle 528.

Still referring to FIG. 14, and as briefly described above, the console assembly 510 includes one or more sensors for monitoring and detecting certain conditions and/or materials stored in the console assembly 510 during use of the delivery device 500. In the present example, the console assembly 510 includes a linear displacement sensor 531 and a radiation sensor 533. The linear displacement sensor 531 is securely attached to the mechanical assembly 529 of the console assembly 510 such that the linear displacement sensor 531 is operable to move within the console assembly 510 in response to an actuation of the handle 528 and a corresponding movement of the vial engagement mechanism 520. The linear displacement sensor 531 is configured to detect and monitor a displacement distance, a velocity of displacement, and/or the like of the handle 528 and the vial engagement mechanism 520.

As will be described in greater detail herein, by measuring a displacement distance or velocity of the handle 528 and/or the vial engagement mechanism 520, computer readable and executable instructions of the delivery device 500, when executed by a processor of the delivery device 500, may determine a flow rate of a fluid media being delivered by the delivery device 500. Additionally or alternatively, the computer readable and executable instructions of the delivery device 500, when executed by a processor of the delivery device 500, may further determine a remaining volume of a fluid media stored within the delivery device 500. As briefly noted above, the data detected by the linear displacement sensor 531 and the information determined by the processor of the delivery device 500 may be displayed at the interface display 530 for operator review.

Still referring to FIG. 14, the radiation sensor 533 is securely attached to the base 512 of the console assembly 510 at a location adjacent to the vial containment region 518. In particular, the radiation sensor 533 is positioned proximate to the sled cavity 532 that is sized and shaped to receive the sled assembly 540 therein. As will be described in greater detail herein, the sled assembly 540 is configured to store and administer therapeutic particles (e.g., radioactive beads, microspheres, medium) therethrough such that the radiation sensor 533 is operable to detect and monitor a radiation level of the therapeutic particles due to a proximate location of the radiation sensor 533 with the sled assembly 540. In particular, the sled assembly 540 is configured to partially receive a vial assembly 580 therein for administering the therapeutic particles from the delivery device 500 and to a patient.

As will further be described herein, by detecting a radiation level of the radioactive medium stored and transferred through the sled assembly 540, computer readable and executable instructions of the delivery device 500, when executed by a processor of the delivery device 500, may determine a radiation dosage delivered from the delivery device 500. Additionally or alternatively, the computer readable and executable instructions executed by a processor of the delivery device 500 may further determine a remaining radiation dosage contained within the delivery device 500 during a procedure. As briefly noted above, the data detected by the radiation sensor 533 and the information determined by the processor of the delivery device 500 may be displayed at the interface display 530 for operator review. It should be understood that in other embodiments the delivery device 500 may include additional or fewer sensors than those shown and described herein (e.g., a dosimeter, a linear encoder, an optical sensor, a linear displacement sensor, a flow sensor, an ultrasonic sensor, a magnetic encoder, a laser distance sensor, an inductance sensor, a radial encoder, a volumetric sensor, mechanical transducers, etc.). A dosimeter and/or radiation sensor of the delivery device 500 may be configured to measure a remaining exposure to ionizing radiation stored within the delivery device 500, and in particularly the sled assembly 540 and/or the vial assembly 580.

As merely illustrative examples only, a linear encoder may be paired with a scale that is configured to encode a position of a remaining dosage of therapeutic particles within the vial assembly 580 such that the linear encoder converts the encoded position into an analog or digital signal that may be decoded into a quantity. An optical sensor/encoder of the delivery device 500 may be configured to convert light rays from within the sled assembly 540 and/or the vial assembly 580 into an electrical signal to measure a physical quantity of light that is thereby translated into a readable form for measuring a remaining radiation dosage contained within the delivery device 500. A magnetic encoder of the delivery device 500 may be configured and operable similar to the optical encoder to determine a remaining radiation dosage but utilizes magnetic fields in lieu of light. An inductive sensor encoder of the delivery device 500 may be configured to utilize electromagnetic induction to detect and measure a remaining dosage stored within the vial assembly 580 by developing a magnetic field therein in response to a current flowing therethrough. A laser distance sensor of the delivery device 500 may be configured to measure a remaining dosage within the vial assembly 580 through transmitting a laser to measure a distance within the vial body 589 to a top liquid surface of the therapeutic particles remaining therein.

By way of further examples, a flow sensor of the delivery device 500 may be positioned in-line with the tubing set of the delivery device 500, and in particular the needle 559, the manifolds 555A, 555B, and/or one or more of the ports 556, and may be configured to measure an amount of fluid (e.g., suspension liquid after the therapeutic particles have effectively mixed with the fluid medium) that passes thereby. An ultrasonic sensor of the delivery device 500 may comprise a transmitter, receiver, and/or transceiver configured to measure a distance to an object (e.g., remaining volume of dosage within the vial assembly 580) based on transmitting ultrasonic signals (i.e. sound waves) therein and measuring an elapsed time before receiving back the bounced sound waves. A radial encoder of the delivery device 500 may comprise an absolute encoder and/or an incremental encoder configured to convert an angular position or motion of the handle 528, the plunger 584, the mechanical assembly 529, and/or other components of the delivery device 500 to analog or digital output signals corresponding to a remaining dosage within the vial assembly 580.

Referring back to FIG. 13, the vial engagement mechanism 520 comprises a pair of lever arms 522 extending outwardly from a neck 524 of the vial engagement mechanism 520, with the neck 524 extending laterally outward from the base 512 of the console assembly 510. The neck 524 of the vial engagement mechanism 520 is disposed within a protective cover 525 such that only the pair of lever arms 522 of the vial engagement mechanism 520 extends through the protective cover 525. The protective cover 525 is operable to shield one or more internal components of the console assembly 510 from an exterior of the console assembly 510, and in particular from the vial containment region 518. As will be described in greater detail herein, the vial containment region 518 of the console assembly 510 is sized and configured to receive one or more radioactive materials therein. In some embodiments, the protective cover 525 of the console assembly 510 may be formed of various materials, including, for example, silicon.

The pair of lever arms 522 is simultaneously movable with the neck 524 of the vial engagement mechanism 520 in response to an actuation of the handle 528 of the console assembly 510. Further, the pair of lever arms 522 are fixed relative to one another such that a spacing formed between the pair of lever arms 522 is relatively fixed. The pair of lever arms 522 of the vial engagement mechanism 520 is configured to securely engage the vial assembly 580 therebetween, and in particular within the spacing formed by the pair of lever arms 522. Accordingly, the vial engagement mechanism 520 is operable to securely attach the vial assembly 580 to the console assembly 510 at the vial containment region 518. Although the vial engagement mechanism 520 is shown and described herein as including a pair of lever arms 522, it should be understood that the vial engagement mechanism 520 may include various other structural configurations suitable for engaging the vial assembly 580.

Still referring to FIG. 13, the console assembly 510 further includes a safety shield 526 secured to the distal end 516 of the base 512 along the vial containment region 518. In particular, the safety shield 526 is a protective covering that is sized and shaped to enclose the vial containment region 518 of the console assembly 510 when secured thereon. The safety shield 526 is selectively attachable to the distal end 516 of the base 512 and is formed of a material that is configured to inhibit radioactive emissions from one or more radioactive doses stored within the vial containment region 518. By way of example only, the safety shield 526 may be formed of acrylonitrile butadiene styrene (ABS), lead, tungsten, tin, pewter, or other suitable materials configured and operable to inhibit radiation emissions. In the present example, the safety shield 526 include a wall thickness of about ⅜ inches. In addition to inhibiting radiation exposure, the safety shield 526 serves as a prevent and contains any spills and/or leaks of radioactive mediums that may occur at the one or more luer connections contained within the vial containment region 518 and between the console assembly 510, the sled assembly 540, and the vial assembly 580. As described in greater detail herein, with the safety shield 526 being selectively attachable to the console assembly 510, the safety shield 526 may be separately cleaned after a use of the delivery device 500 during a procedure.

In other embodiments, the delivery device 500 may include a splash guard in addition to and/or in lieu of the safety shield 526. The splash guard may be formed of a non-opaque housing that encloses the vial containment region 518, similar to the safety shield 526, and may be selectively opened and closed through various mechanisms. For example, in some embodiments the splash guard may include a sliding window, a hinge coupling to the console assembly 510 such that the splash guard is pivotable, and/or the like. The splash guard may be formed of various polymers, including, but not limited to, polycarbonate. It should be understood that the splash guard may serve to provide a protective shielding against spills and/or leaks during a loading of the sled assembly 540 and/or the vial assembly 580 to the console assembly 510 during a preparation of the delivery device 500 for use in a procedure.

The distal end 516 of the console assembly 510 further includes a sled cavity 532 that is sized and shaped to receive the sled assembly 540 therein. The sled cavity 532 includes a pair of alignment features 534 extending therein, with the alignment features 534 sized and shaped to correspond with complimentary alignment features of the sled assembly 540 (e.g., alignment ribs 554) to thereby facilitate a coupling of the sled assembly 540 with the base 512 of the console assembly 510 within the sled cavity 532. In the present example, the pair of alignment features 534 comprise longitudinal recesses extending laterally along the sled cavity 532, however, it should be understood that the pair of alignment features 534 may take various other forms and configurations than those shown and described herein without departing from the scope of the present disclosure. For example, the alignment features of the console assembly 510 may include one or more magnets that are configured to mate with corresponding magnets of the sled assembly 540.

Still referring to FIG. 13, the sled assembly 540 is configured to partially receive a vial assembly 580 therein for administering therapeutic particles (e.g., radioactive fluid medium) from the delivery device 500 and to a patient. In particular, the sled assembly 540 comprises a proximal end 542 and a distal end 544 with a pair of sidewalls 546 extending therebetween. The proximal end 542 of the sled assembly 540 includes a handle 552 extending proximally therefrom. The handle 552 is configured to facilitate movement of the sled assembly 540, and in particular, an insertion of the sled assembly 540 into the sled cavity 532 of the console assembly 510. The proximal end 542 further includes one or more ports 556 for coupling one or more delivery lines (i.e., tubing) to the sled assembly 540. With the one or more delivery lines further be coupled to one or more external devices at an end of the line opposite of the ports 556, the ports 556 effectively serve to fluidly couple the sled assembly 540 to the one or more external devices via the delivery lines connected thereto. The pair of sidewalls 546 of the sled assembly 540 includes at least one alignment rib 554 extending laterally outward therefrom, where the alignment ribs 554 are sized and shaped to correspond with and mate to the pair of alignment features 534 of the console assembly 510. Accordingly, the pair of alignment ribs 554 are configured to facilitate an alignment and engagement of the sled assembly 540 with the console assembly 510 when the distal end 544 is slidably received within the sled cavity 532 of the base 512. As will be described in greater detail herein, the pair of alignment features 534 and the pair of alignment ribs 554 are operable to inhibit a vertical deflection and/or movement of the sled assembly 540 during use of the delivery device 500, and more specifically, during a vertical translation of the vial engagement mechanism 520 and a corresponding vertical retraction of the vial assembly 580 that is received within the sled assembly 540.

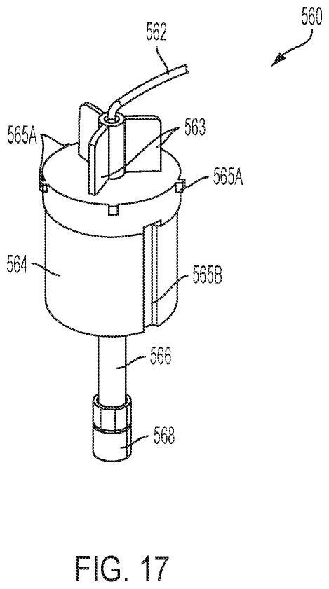

The sled assembly 540 further includes a top surface 548 extending from the proximal end 542 and the distal end 544 and positioned between the pair of sidewalls 546. The top surface 548 of the sled assembly includes a recessed region 549 and a locking system 550. The recessed region 549 is sized and shaped to form a recess and/or cavity along the top surface 548, where the recessed region 549 is capable of receiving and/or collecting various materials therein, including, for example, leaks of various fluid media during use of the delivery device 500. The locking system 550 of the sled assembly 540 forms an opening along the top surface 548 that is sized and shaped to receive one or more devices therein, such as a priming assembly 560 and a vial assembly 580 (See FIG. 17). In some embodiments, the sled assembly 540 comes preloaded with the priming assembly 560 disposed within the locking system 550. The priming assembly 560 includes a priming line 562 extending outwardly from the locking system 550 of the sled assembly 540. As will be described in greater detail herein, the priming assembly 560 serves to purge the delivery device 500 of air prior to utilizing the delivery device 500 in a procedure.

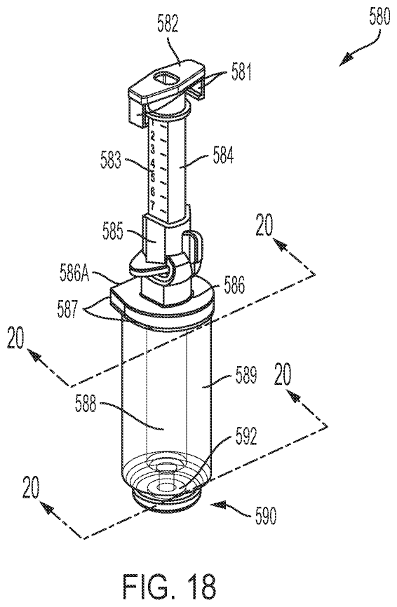

Referring now to FIG. 15, the locking system 550 includes an annular array of projections 551 extending outwardly therefrom, and in particular, extending laterally into the aperture formed by the locking system 550 along the top surface 548. The annular array of projections 551 are formed within an inner perimeter of the locking system 550 and extend along at least two sequentially-arranged rows. As will be described in greater detail herein, the annular array of projections 551 included in the locking system 550 are configured to engage a corresponding locking feature 586 of the vial assembly 580 (See FIG. 18) to thereby securely fasten the vial assembly 580 to the sled assembly 540. It should be understood that the multiple rows of projections 551 of the locking system 550 serve to provide a double-locking system to ensure the sled assembly 540, and in particular a needle 559 of the sled assembly 540, is securely maintained through a septum 592 of the vial assembly 580 (See FIG. 18) during use of the delivery device 500 in a procedure. Accordingly, the double-locking system formed by the locking system 550 reduces occurrences of unintended delivery of a dose during preparation of the delivery device 500 for a procedure. It should further be understood that additional and/or fewer projections 551 may be included along the locking system 550 than those shown and described herein without departing from the scope of the present disclosure. Alternatively, in other embodiments the locking system 550 may include various other suitable engagement features, other than the annular array of projections 551 shown and described herein, that are configured and operable to a snap-fit engagement with the vial assembly 580. For example, in other embodiments the locking system 550 may comprise a threaded portion, one or more magnets, one or more crush ribs, and/or the like.

The sled assembly 540 further includes a vial chamber 558 that is sized and shaped to receive the priming assembly 560 and the vial assembly 580 therein, respectively. In other words, the vial chamber 558 is sized to individually receive both the priming assembly 560 and the vial assembly 580 separate from one another. The vial chamber 558 is encapsulated around a protective chamber or shield 557 disposed about the vial chamber 558. The protective shield 557 is formed of a material configured to inhibit radioactive emissions from extending outwardly from the vial chamber 558, such as, for example, a metal. Additionally, the sled assembly 540 includes a needle extending through the protective shield 557 and into the vial chamber 558 along a bottom end of the vial chamber 558. The needle 559 is fixedly secured relative to the vial chamber 558 such that any devices received through the aperture of the locking system 550 and into the vial chamber 558 are to encounter and interact with the needle 559 (e.g., the priming assembly 560, the vial assembly 580, and the like).

Still referring to FIG. 15, the needle 559 is coupled to a distal manifold 555A and a proximal manifold 555B disposed within the sled assembly 540, and in particular the manifold 555A, 555B is positioned beneath the vial chamber 558 and the protective shield 557. The proximal manifold 555B is fluidly coupled to the needle 559 and the distal manifold 555A is fluidly coupled to the one or more ports 556 of the sled assembly 540. The proximal manifold 555B is in fluid communication with the distal manifold 555A through a one-way check valve 553 disposed therebetween. It should be understood that the one-way check valve 553 is configured to facilitate fluid communication from the proximal manifold 555B to the distal manifold 555A and prevent fluid communication from the distal manifold 555A to the proximal manifold 555B. In other words, the one-way check valve 553 prevents a backflow of fluid into the sled assembly 540 and/or the vial assembly 580 coupled thereto.

Accordingly, the proximal manifold 555B is in fluid communication with the one or more ports 556 via the distal manifold 555A, however, the one or more ports 556 are not in fluid communication with the proximal manifold 555B due to a position of the one-way check valve 553 disposed between the manifolds 555A, 555B. Thus, the needle 559 is in fluid communication with the one or more delivery lines and/or devices coupled to the sled assembly 540 at the one or more ports 556 via the manifolds 555A, 555B secured therebetween. As will be described in greater detail herein, the one or more ports 556 of the sled assembly 540 may be coupled to a bag (e.g., saline bag), a syringe, a catheter, and/or the like via one or more delivery lines coupled thereto. In other embodiments, the needle 559 may be omitted entirely for an alternative device, such as, for example, a valve system, a needleless injection port, and/or the like.

Still referring to FIG. 15, the sled assembly 540 includes a removable battery pack 570 coupled to the sled assembly 540 along the distal end 544. The removable battery pack 570 comprises a battery 572, electrical contacts 574, and a removable tab 576. It should be understood that in some embodiments the removable battery pack 570 may be preloaded onto the sled assembly 540 while in other embodiments the removable battery pack 570 is separate from the sled assembly 540 such that an operator is required to couple the removable battery pack 570 to the sled assembly 540 along the distal end 544. In either instance, the battery 572 of the delivery device 500 is isolated from one or more fluid paths and radiation sources due to a location of the battery 572 in the removable battery pack 570.

The battery 572 may comprise various quantities and types of batteries for powering the delivery device 500, such as, for example, four (4) disposable double-A (AA) batteries, alkaline batteries, Li-ion batteries, mignon batteries, single cell dry batteries, and/or the like. In some embodiments, the battery 572 may be encapsulated in a polymer or wax material. As will be described in greater detail herein, the electrical contacts 574 of the removable battery pack 570 extend outwardly from the removable battery pack 570 and are operable to contact against and interact with corresponding electrical contacts 511 of the console assembly 510 (See FIG. 13) when the sled assembly 540 is coupled to the base 512 at the sled cavity 532. Accordingly, the removable battery pack 570 is operable to provide electrical power to the delivery device 500, and in particular the console assembly 510, when the sled assembly 540 is coupled to the console assembly 510.

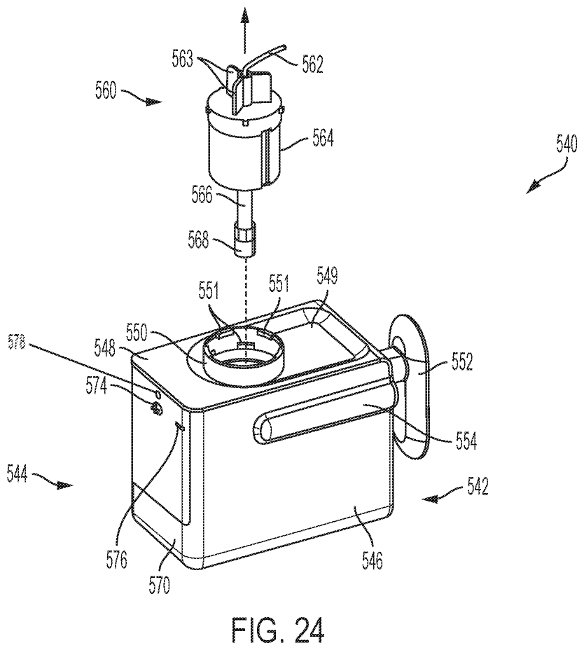

Still referring to FIG. 15, the removable tab 576 of the removable battery pack 570 is selectively removable from the removable battery pack 570. The removable tab 576 is operable to check a battery status of the removable battery pack 570 upon removal of the removable tab 576. As will be described in greater detail herein, removal of the removable tab 576 prior to a commencement of a procedure with the delivery device 500 provides an operator of the delivery device 500 an indication of whether the removable battery pack 570 contains sufficient power stored therein to perform a procedure. The removable battery pack 570 generates a feedback indicating a sufficiency of the battery 572 in response to a removal of the removable tab 576. For example, in some embodiments the removable battery pack 570 includes a LED status indicator 578 (see FIG. 24) that visually displays a color indicative of a battery power of the battery 572 (e.g., green, yellow, red). In other embodiments, the removable battery pack 570 may include a speaker that generates an audible alert indicative of a battery power of the battery 572. It should be understood that in other embodiments the sled assembly 540 and/or the console assembly 510 may be electrically powered by various other suitable power sources without departing from the scope of the present disclosure. For example, one or more of the sled assembly 540 and/or the console assembly 510 may be directly coupled to an external power supply, the console assembly 510 may include one or more batteries stored therein, and/or the like.

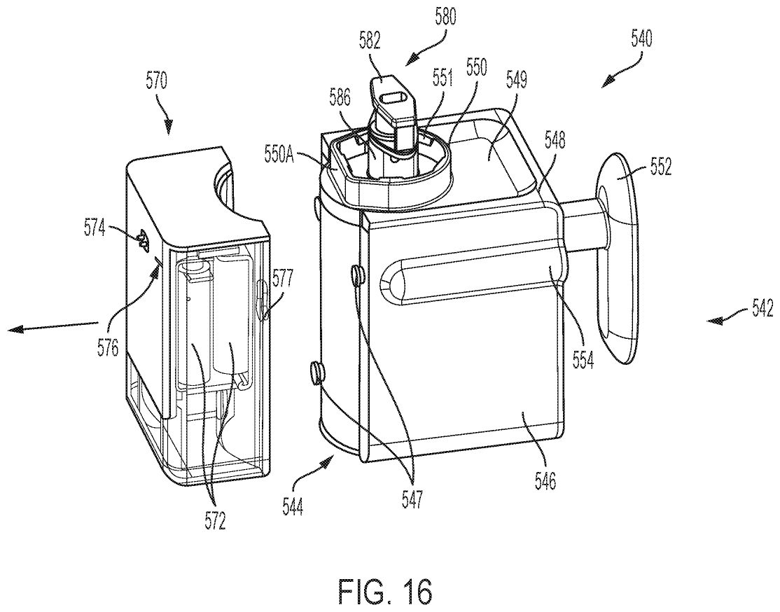

Referring now to FIG. 16, the sled assembly 540 includes one or more retention features 547 disposed along the distal end 544 of the sled assembly 540 for securing the removable battery pack 570 thereto. In particular, the retention features 547 of the sled assembly 540 comprise protrusions extending outwardly from the distal end 544. The removable battery pack 570 includes one or more corresponding retention features 577 disposed along a surface of the removable battery pack 570 opposite of the electrical contacts 574, where the corresponding retention features 577 of the removable battery pack 570 are configured to engage the retention features 547 of the sled assembly 540. In particular, the retention features 577 of the removable battery pack 570 comprise recesses extending inwardly into the removable battery pack 570 to receive the retention features 547 of the sled assembly 540 therein, to thereby securely couple the removable battery pack 570 to the sled assembly 540 at the distal end 544. It should be understood that various other retention features 547, 577 may be included in the sled assembly 540 and the removable battery pack 570 than those shown and described herein without departing from the scope of the present disclosure. For example, corresponding retention features may comprise magnets, snaps, threads, and/or the like.

Additionally, as will be described in greater detail herein, in some embodiments the locking system 550 may include at least one planar wall 550A relative to a remaining circular orientation of the locking system 550. In this instance, an aperture formed by the locking system 550 through the top surface 548 of the sled assembly 540 is irregularly-shaped, rather than circularly-shaped as shown and described above. In this instance, the vial assembly 580 includes an locking feature 586 that has a shape and size that corresponds to the locking system 550, and in particular the at least one planar wall 550A such that the vial assembly 580 is received within the sled assembly 540 only when an orientation of the vial assembly 580 corresponds with an alignment of the locking feature 586 and the locking system 550. In other words, a corresponding planar wall 586A of the locking feature 586 (See FIG. 18) must be aligned with the planar wall 550A of the locking system 550 for the vial assembly 580 to be receivable within an aperture formed by the locking system 550 of the sled assembly 540.

Referring now to FIG. 17, the priming assembly 560 of the delivery device 500 is depicted. The priming assembly 560 comprises the priming line 562, a handle 563, a central body 564, an elongated shaft 566, and a needle end 568. The central body 564 is sized and shaped to be slidably received within the vial chamber 558 of the sled assembly 540, and in particular, includes a diameter that is substantially similar to a diameter of the vial chamber 558 such that a press-fit is formed between the central body 564 and the vial chamber 558 when the priming assembly 560 is received within the sled assembly 540. The handle 563 and the elongated shaft 566 are integrally formed with the central body 564, with the handle 563 extending vertically outward from the central body 564 at an end opposite of the elongated shaft 566.

In other words, the handle 563 extends relatively upward from the central body 564 and the elongated shaft 566 extends relatively downward from the central body 564, in a direction opposite of the handle 563. Accordingly, when the priming assembly 560 is slidably received within the vial chamber 558 of the sled assembly 540, the handle 563 is positioned adjacent to the top surface 548 of the sled assembly 540 and the elongated shaft 566 is disposed within the sled assembly 540. The handle 563 is configured to facilitate grasping and maneuvering the priming assembly 560 for insertion into and extraction out of the sled assembly 540, respectively. It should be understood that in other embodiments the handle 563, the central body 564, and/or the elongated shaft 566 may be separate components assembled together to form the priming assembly 560.

Still referring to FIG. 17, the elongated shaft 566 is sized at a predetermined length to thereby position the central body 564 and the handle 563 of the priming assembly 560 proximate to the aperture formed by the locking system 550 of the sled assembly 540. In this instance, the handle 563 may be readily accessible to an operator of the delivery device 500 via the aperture formed by the locking system 550. It should be understood that a collective longitudinal length of the handle 563, the central body 564, and the elongated shaft 566 is substantially similar to a longitudinal length of the vial chamber 558 of the sled assembly 540 such that the handle 563 is partially disposed within the vial chamber 558 and/or partially exposed from the vial chamber 558 (See FIG. 13).

The elongated shaft 566 of the priming assembly 560 further includes the needle end 568 positioned along a terminal end of the elongated shaft 566 opposite of the central body 564. The needle end 568 is formed of a material that is operable to receive the needle 559 of the sled assembly 540 therethrough in response to the priming assembly 560 being received within the vial chamber 558 of the sled assembly 540. For example, the needle end 568 of the priming assembly 560 may be formed of an elastomer material that is configured to be punctured by the needle 559 when the needle end 568 is slidably inserted through the vial chamber 558 and positioned against the needle 559. In the present example, the priming assembly 560 further includes one or more alignment features 565A, 565B positioned along the central body 564 that are configured to maintain the priming assembly 560 in the vial chamber 558 of the sled assembly 540.

Referring now to FIG. 18, the vial assembly 580 of the delivery device 500 is depicted. The vial assembly 580 comprises an engagement head 582, a plunger 584, an locking feature 586, and a vial body 589. In particular, the engagement head 582 of the vial assembly 580 is positioned at a terminal end of the plunger 584 opposite of the locking feature 586 and the vial body 589. The engagement head 582 includes a pair of arms 581 extending laterally outward relative to a longitudinal length of the plunger 584 extending downwardly therefrom. In the present example, the engagement head 582 is integrally formed with the plunger 584, however, it should be understood that in other embodiments the engagement head 582 and the plunger 584 may be separate features fastened thereto. In either instance, the engagement head 582 and the plunger 584 is movable relative to the locking feature 586 and the vial body 589 such that the engagement head 582 and the plunger 584 are slidably translatable through the locking feature 586 and the vial body 589. In particular, as will be described in greater detail herein, the plunger 584 may translate into and out of an internal chamber 588 of the vial body 589 in response to a linear translation of the vial engagement mechanism 520 when the engagement head 582 is secured to the pair of lever arms 522.

The plunger 584 includes a plurality of indicia and/or markings 583 positioned along a longitudinal length of the plunger 584. The plurality of markings 583 is indicative of a relative extension of the engagement head 582 and the plunger 584 from the locking feature 586 and the vial body 589. As briefly noted above, the engagement head 582 is configured to attach the vial assembly 580 to the vial engagement mechanism 520. In particular, the pair of arms 581 of the engagement head 582 are sized and shaped to couple with the pair of lever arms 522 of the vial engagement mechanism 520 when the vial assembly 580 is received within the sled assembly 540 and the sled assembly is inserted into the sled cavity 532 of the console assembly 510. As will be described in greater detail herein, the pair of lever arms 522 are received between the pair of arms 581 of the engagement head 582 and the plunger 584 in response to a predetermined translation force applied to the vial engagement mechanism 520. The engagement head 582 and the plunger 584 may be formed of various materials, including, but not limited to, a metal, plastic, and/or the like.

Still referring to FIG. 18, the vial assembly 580 further includes a safety tab 585 coupled to the plunger 584 relatively above the locking feature 586 and below the engagement head 582 such that the safety tab 585 is positioned along the longitudinal length of the plunger 584. The safety tab 585 may be formed of various materials, such as, for example, a plastic, and is preassembled onto the vial assembly 580 prior to a use of the delivery device 500. The safety tab 585 is removably fastened to the plunger 584 and inhibits the plunger 584 from translating relative to the vial body 589. In particular, the safety tab 585 abuts against the locking feature 586 in response to an application of linear force onto the plunger 584 to translate the plunger 584 relatively downward into the vial body 589. In this instance, the safety tab 585 is configured to inhibit an inadvertent movement of the plunger 584, and in response, an inadvertent delivery of a fluid media stored within the internal chamber 588 of the vial body 589 (e.g., therapeutic particles, radioembolizing beads). As will be described in greater detail herein, the safety tab 585 is selectively disengaged from the plunger 584 in response to a coupling of the vial assembly 580 with the vial engagement mechanism 520, and in particular an engagement of the pair of lever arms 522 with the engagement head 582.

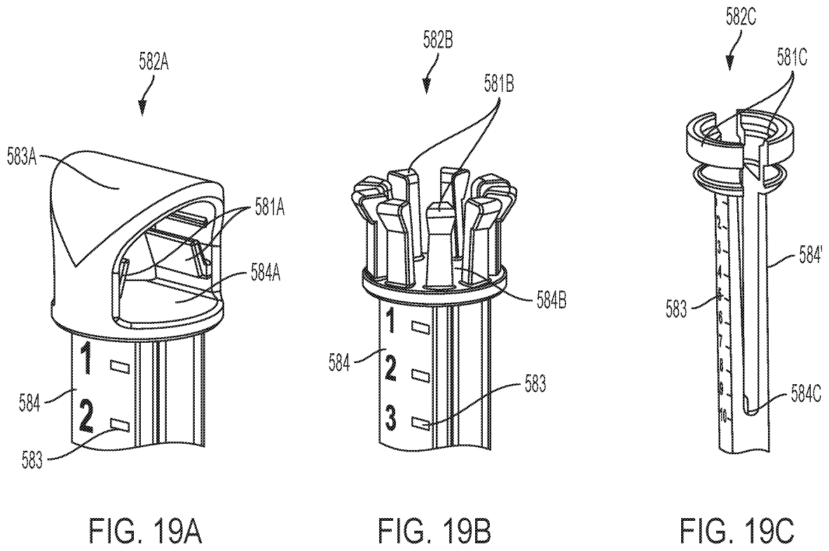

Although the engagement head 582 of the vial assembly 580 is shown and described herein as including a pair of arms 581 extending laterally outward from the plunger 584, it should be understood that the engagement head 582 may include various other structural configurations suitable for engaging the pair of lever arms 522 of the vial engagement mechanism 520. For example, referring now to FIGS. 19A-19C, alternative embodiments of an engagement head of the vial assembly 580 are depicted. It should be understood that the engagement heads shown and described herein may be similarly incorporated onto the vial assembly 580 as the engagement head 582 described above.

Referring now to FIG. 19A, an alternative embodiment of an engagement head 582A is depicted. The engagement head 852A comprises a ring 583A that defines an aperture with a top planar surface 584A of the plunger 584. The ring 583A includes at least a pair of flexible tabs (resilient arms) 581A extending laterally inwardly from the ring 583A and into the aperture formed therein. In particular, the pair of flexible tabs 581A is separated from one another at opposing sides of the ring 583A and are angled relative inward toward one another. In this instance, the pair of flexible tabs 581A is transverse relative to a longitudinal length of the plunger 584. In the present example, the pair of flexible tabs 581A is manually flexible such that the pair of flexible tabs 581A may be selectively compressed outwardly away from one another when an inward force is applied thereto (e.g., from the pair of lever arms 522 received through the ring 583A). The pair of flexible tabs 581A is resiliently biased to expand outward relative to one another and into the aperture defined by the ring 583A in a default state.

In the present example, the pair of lever arms 522 of the vial engagement mechanism 520 may be received through the aperture formed by the ring 583A such that the pair of lever arms 522 are positioned between, and engaged against, the pair of flexible tabs 581A. In this instance, the engagement head 582A is securely fastened to the vial engagement mechanism 520. It should be understood that the engagement head 582A of the present example may be configured and operable to correspond to alternative embodiments of a vial engagement mechanism that includes features distinct from the pair of lever arms 522 of the vial engagement mechanism 520 shown and described above.

Referring now to FIG. 19B, an alternative engagement head 582B is depicted. The engagement head 582B comprises a plurality of flexible fingers 581B extending vertically-upward from the plunger 584. In particular, the plurality of flexible fingers 581B extends parallel to, and in coaxial alignment with, a longitudinal length of the plunger 584. A terminal end of each of the plurality of flexible fingers 581B is positioned relatively above a top planar surface 584B of the plunger 584. In the present example, the plurality of flexible fingers 581B is manually flexible such that the plurality of flexible fingers 581B may be selectively compressed inward toward one another when an outward force is applied thereto. The plurality of flexible fingers 581B is resiliently biased to expand outward away from one another in a default state.

Accordingly, inserting the engagement head 582B into the vial engagement mechanism 520, and in particular between the pair of lever arms 522 of the vial engagement mechanism 520, causes the plurality of flexible fingers 581B to be compressed inwardly and thereby engage against the pair of lever arms 522 that are disposed about the plurality of flexible fingers 581B. In this instance, the plurality of flexible fingers 581B is securely fastened to the vial engagement mechanism 520 through an outward expansion of the flexible fingers 851B against the pair of lever arms 522. It should be understood that the engagement head 582B of the present example may be configured and operable to correspond to alternative embodiments of a vial engagement mechanism that includes features distinct from the pair of lever arms 522 of the vial engagement mechanism 520 shown and described above.

Referring now to FIG. 19C, another alternative engagement head 582C is depicted. The engagement head 582C comprises a pair of flexible clamps 581C positioned above a plunger 584′. The plunger 584′ of the present embodiment is different than the plunger 584 of the prior embodiments in that the plunger 584′ is bifurcated along a longitudinal length of the plunger 584′ with the bifurcation extending from the pair of flexible clamps 581C of the engagement head 582C to a terminal end 584C. The pair of flexible claims 581C extends parallel to, and in coaxial alignment with, a longitudinal length of the plunger 584. In the present example, the pair of flexible clamps 581C is manually flexible such that the pair of flexible clamps 581C may be selectively compressed inward toward one another when an outward force is applied thereto. The pair of flexible clamps 581C is resiliently biased to expand outward away from one another in a default state.

Accordingly, inserting the engagement head 582C into the vial engagement mechanism 520, and in particular between the pair of lever arms 522 of the vial engagement mechanism 520, causes the pair of flexible clamps 581C to be compressed inwardly and thereby engage against the pair of lever arms 522 that are disposed about the pair of flexible clamps 581C. In this instance, the pair of flexible clamps 581C is securely fastened to the vial engagement mechanism 520 through an outward expansion of the flexible clamps 581C against the pair of lever arms 522. It should be understood that the engagement head 582C of the present example may be configured and operable to correspond to alternative embodiments of a vial engagement mechanism that includes features distinct from the pair of lever arms 522 of the vial engagement mechanism 520 shown and described above. It should further be understood that various other configurations and geometries of an engagement head may be incorporated with the vial assembly 580 without departing from the scope of the present disclosure. For example, in others embodiments the engagement head of the vial assembly 580 may comprise one or more magnets, threads, cams, and/or the like.

Referring back to FIG. 18, the locking feature 586 extends about a top end of the vial body 589. In the present example, the locking feature 586 of the vial assembly 580 comprises a bushing that defines a lateral edge 587 extending laterally outward along an outer perimeter of the locking feature 586. The lateral edge 587 of the locking feature 586 is sized and shaped to engage the annular array of projections 551 of the locking system 550 when the vial assembly 580 is received within the vial chamber 558 of the sled assembly 540. As will be described in greater detail herein, the locking feature 586, and in particular the lateral edge 587 of the locking feature 586, is configured to securely fasten the vial assembly 580 to the locking system 550 to inhibit removal of the vial body 589 from the vial chamber 558 of the sled assembly 540 during use of the delivery device 500 in a procedure. In some embodiments, as briefly described above, the locking feature 586 includes at least one planar wall 586A such that the locking feature 586 comprises an irregular-profile. The at least one planar wall 586A is configured to correspond to the planar wall 550A of the locking system 550 such that an alignment of the planar walls 550A, 586A is required for the vial assembly 580 to be received through an aperture formed by the locking system 550.

It should be understood the planar walls 550A, 550B serve to ensure that the safety tab 585 of the vial assembly 580 is coupled to the plunger 584 in a manner that allows for a removal of the safety tab 585 by the vial engagement mechanism 520. In particular, the pair of lever arms 522 of the vial engagement mechanism 520 is configured to exert a lateral force against the safety tab 585 in response to the sled assembly 540 being slidably received within the sled cavity 532. Accordingly, an orientation of the safety tab 585 relative to the pair of lever arms 522 may be facilitated to ensure ease of removal of the safety tab 585, when the sled assembly 540 is coupled to the console assembly 510, by requiring a proper alignment of the vial assembly 580 with the locking system 550 when the vial assembly is coupled to the sled assembly 540.

Still referring to FIG. 18, the vial body 589 extends downwardly relative from the locking feature 586 and has a longitudinal length that is sized to receive at least a portion of a longitudinal length of the plunger 584 therein. By way of example only, a longitudinal length of the vial body 589 may be about 8 millimeters to about 10 millimeters, and in the present example comprises 9 millimeters, while a longitudinal length of the plunger 584 may be about 9 millimeters to about 11 millimeters, and in the present example comprises 10 millimeters. Accordingly, in some embodiments a longitudinal length of the plunger 584 exceed a longitudinal length of the vial body 589 such that a translation of the plunger 584 into the internal chamber 588 of the vial body 589 causes a fluid media stored therein to be transferred outward from the vial body 589. As will be described in greater detail herein, a translation of the plunger 584 through the internal chamber 588 of the vial body 589 provides for an administration of a fluid media stored within the vial body 589 outward from the vial assembly 580. The vial body 589 may be formed of various materials, including, for example, a thermoplastic polymer, copolyester, polycarbonate, a biocompatible plastic, polysulfone, ceramics, metals, and/or the like.

The vial body 589 is of the present example is formed of a material that is configured to inhibit radioactive emissions from a fluid media stored within the internal chamber 588 of the vial body 589. For example, the vial body 589 may be formed of a plastic, such as polycarbonate, and have a width of approximately 9 millimeters (mm). A density and material composition of the vial body 589 may collectively inhibit gamma radiation emission from electron particles stored within the internal chamber 588. In the present example, a chemical composition of the plastic of the vial body 589, along with the 9 mm wall thickness, provides a plurality of atoms disposed within the vial body 589 that are capable of encountering the electron particles generating beta radiation and reducing an emission of said radiation from the vial assembly 580. Accordingly, the vial assembly 580 allows an operator to handle the radioactive material stored within the vial body 589 without being exposed to beta radiation. It should be understood that various other materials and/or wall sections may be incorporated in the vial body 589 of the vial assembly 580 in other embodiments without departing from the scope of the present disclosure.

Still referring to FIG. 18, the vial body 589 of the vial assembly 580 is sealed at a first terminal end by the locking feature 586. The vial assembly 580 further includes a cap 590 positioned at an opposing, terminal end of the vial body 589 opposite of the locking feature 586, such that the cap 590 seals a second terminal end of the vial body 589 of the vial assembly 580. Additionally, the vial assembly 580 includes a septum 592 positioned adjacent to the cap 590 and in fluid communication with a terminal end of the vial body 589 opposite of the locking feature 586. The septum 592 forms a seal against a terminal end of the vial body 589 and the cap 590 retains the septum 592 therein. The septum 592 may be formed of various materials, including, for example, an elastomer, silicon, bromobutyl elastomer, rubber, urethanes, and/or the like. The septum 592 is configured to provide an air-tight seal for the vial body 589 to thereby inhibit a release of a fluid media stored therein (e.g., radioembolizing beads). As will be described in greater detail herein, the septum 592 of the vial assembly 580 is configured to be punctured by the needle 559 of the sled assembly 540 when the vial assembly 580 is received within the vial chamber 558, thereby establishing fluid communication between the vial body 589 and the sled assembly 540. In other embodiments, the septum 592 may be omitted entirely for an alternative device, such as, for example, a valve system, needle injection port, and/or the like.

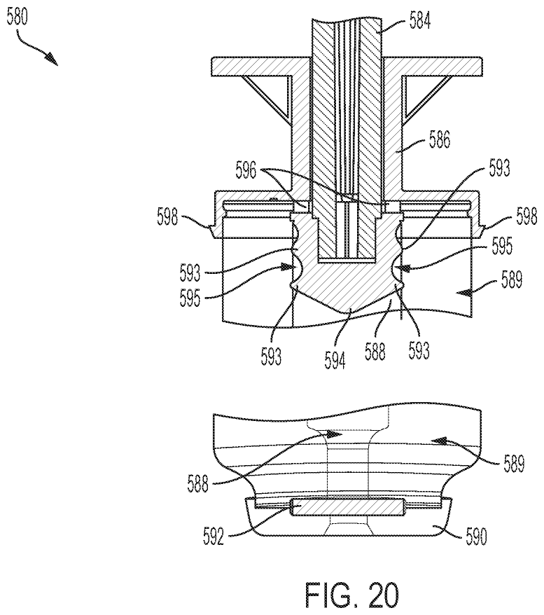

Referring to FIG. 20, the vial assembly 580 further includes a stopper 594 fixedly coupled to a terminal end of the plunger 584 opposite of the engagement head 582. In this instance, with the plunger 584 coupled to, and slidably translatable through, the internal chamber 588 of the vial body 589, the stopper 594 is effectively disposed within the vial body 589. Accordingly, it should be understood that the stopper 594 is sized and shaped in accordance with a size (e.g., a diameter) of the internal chamber 588 of the vial body 589. The stopper 594 is secured to the plunger 584 such that the stopper 594 is slidably translatable through the vial body 589 in response to a translation of the plunger 584 through the vial body 589. The stopper 594 is defined by two or more ribs 593 extending laterally outward and one or more troughs 595 defined between at least two ribs 593. In the present example, the stopper 594 includes six ribs 593 and two cavities formed therebetween, however, it should be understood that additional and/or fewer ribs 593 and troughs 595 may be included in the stopper 594 in other embodiments.

The stopper 594 is configured to form a liquid-seal against the internal chamber 588 of the vial body 589, and is formed of a various polymers with a predetermined viscoelasticity. For example, in some embodiments the stopper 594 is formed of an elastomer, silicone, rubber, urethane, plastic, polyethylene, polypropylene, and/or the like. In this instance, the stopper 594 is operable to inhibit a fluid media stored within the vial body 589 from extending (i.e., leaking) past the stopper 594 and out of the vial body 589. In particular, the two or more ribs 593 of the stopper 594 abut against, and form a seal along, the internal chamber 588 of the vial body 589 to thereby inhibit a fluid media from passing beyond the ribs 593. The one or more troughs 595 formed between the two or more ribs 593 of the stopper 594 are configured to receive, and more specifically capture, any fluid media that may inadvertently extend (i.e., leak) beyond the ribs 593 of the stopper 594. Accordingly, the one or more troughs 595 serve as a safety mechanism of the vial assembly 580 to ensure a fluid media is maintained within the vial body 589 and not exposed beyond the vial assembly 580.

Still referring to FIG. 20, the two or more ribs 593 of the stopper 594 are additionally configured to push a fluid media stored within the vial body 589 in one or more directions therein (e.g., toward the cap 590) in response to a translation of the plunger 584. With the ribs 593 of the stopper 594 pressed against the internal chamber 588 of the vial body 589, translation of the plunger 584 provides for a translation of the ribs 593 against and along the internal chamber 588 of the vial body 589 such that any fluid media located in front (i.e., beneath) of the stopper 594 is effectively redirected within the vial body 589 in a direction of travel of the plunger 584 and the stopper 594. The vial assembly 580 further includes an annular washer 596 disposed within the vial body 589. In particular, the annular washer 596 is securely fixed to the plunger 584 adjacent to the stopper 594, which is secured to the plunger 584 at a terminal end opposite of the engagement head 582. Accordingly, the annular washer 596 is secured to the plunger 584 and disposed within the vial body 589 adjacent to the stopper 594. With the annular washer 596 secured to the plunger 584 adjacent to the stopper 594, the annular washer 596 is effectively disposed within the vial body 589.

The annular washer 596 may be formed of various materials, including, for example, a plastic, metal, and/or the like. The annular washer 596 may be fixedly secured to the plunger 584 via various suitable means, including, for example, by an adhesive. It should be understood that the annular washer 596 is sized and shaped in accordance with a size (e.g., a diameter) of the internal chamber 588 of the vial body 589 such that the annular washer 596 slidably translates within the internal chamber 588 of the vial body 589 simultaneous with the plunger 584 and the stopper 594. The annular washer 596 is configured to inhibit a removal of the plunger 584 from the vial body 589 by abutting against a bottom end of the locking feature 586 when the plunger 584 is translated relatively outward (i.e., upward) to a fullest extent. In other words, with the annular washer 596 securely fixed to a terminal end of the plunger 584 that is disposed within the vial body 589, and with the plunger 584 having a size that is smaller than the vial body 589 to allow for a translation of the plunger 584 therethrough, the annular washer 596 serves to form an impediment for the plunger 584 to be translated outward of the vial body 589. The annular washer 596 is configured to engage a bottom end of the locking feature 586 in response to a retraction of the plunger 584 from the vial body 589 at a predetermined distance (i.e., predetermined length of the plunger 584).

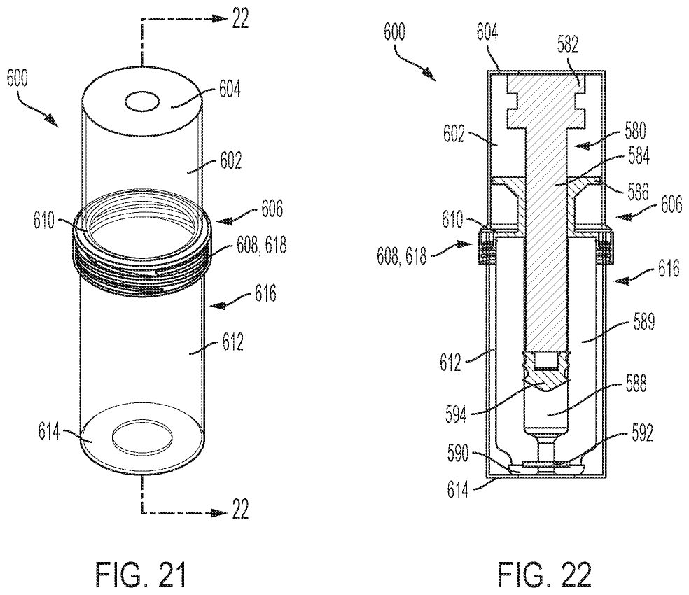

Referring now to FIG. 21, a sterile container assembly 600 is depicted. The sterile container assembly 600 is sized and shaped to receive the vial assembly 580 therein for storing and transporting the vial assembly 580 prior to use of the vial assembly 580 while maintaining a sterility of the vial assembly 580. The sterile container assembly 600 comprises a top housing 602 including a closed end 604 and an open end 606, and a bottom housing 612 including a closed end 614 and an open end 616. The closed ends 604, 614 of both housings 602, 612 of the sterile container assembly 600 include a material that is operable to form a liquid seal, such as, for example, a synthetic material, polyethylene fiber, and/or the like. The seal formed at the closed ends 604, 614 of both housings 602, 612 are configured to permit steam penetration therethrough for sterilization of the contents of the housings 602, 612.

The open ends 606, 616 of both housings 602, 612 include corresponding mating system 608, 618 that are configured to couple the housings 602, 612 to one another. In the present example, the mating systems 608, 618 of the sterile container assembly 600 are corresponding threaded portions positioned along the open ends 606, 616 of each of the housings 602, 612 such that the threaded portions are configured to mesh with one another to secure the top housing 602 to the bottom housing 612. It should be understood that various other mating systems 608, 618 may be incorporated with the sterile container assembly 600 without departing from the scope of the present disclosure, such as, for example, magnets, elastics, snaps, and/or the like. The sterile container assembly 600 may be formed of various materials, including, but not limited to, a metal, plastic, and/or the like. The sterile container assembly 600 is configured and operable to inhibit leaks of therapeutic particles externally therefrom when the top housing 602 is coupled to the bottom housing 612 due to the liquid seals formed along the closed ends 604, 614 and the gasket seal 610 formed between the open ends 606, 616.

Referring now to FIG. 22, the vial assembly 580 is depicted as being received within the sterile container assembly 600. In particular, the cap 590 of the vial assembly 580 is received at and positioned proximate to the closed end 614 of the bottom housing 612 of the sterile container assembly 600. Further, the engagement head 582 of the vial assembly 580 is received at and positioned proximate to the closed end 604 of the top housing 602 of the sterile container assembly 600. In this instance, the open ends 606, 616 of the housings 602, 612 of the sterile container assembly 600 are secured to one another via the corresponding mating systems 608, 618 of each of the housings 602, 612. In some embodiments, at least one of the top housing 602 and/or the bottom housing 612 includes a gasket seal adjacent to the open end 606, 616 such that a seal is formed proximate to the mating systems 608, 618 when the top housing 602 is coupled to the bottom housing 612. In the present example, the top housing 602 includes an annular gasket seal 610 extending within the top housing 602 adjacent to the open end 606, and in particular, along the mating system 608 of the top housing 602. The gasket seal 610 is configured to form an airtight seal between the housings 602, 612 of the sterile container assembly 600 when the mating systems 608, 618 are coupled thereto.

In other embodiments, the vial assembly 580 may be stored and transferred to the delivery device 500 via a loading system (not shown). The loading system may include a radiation shielding and is configured to hold the vial assembly 580 therein. The loading system may include a removable sled that may be aligned with the vial engagement mechanism 520 of the console assembly 510, where the sled includes one or more plates for providing radiation shielding that are formed of various materials, including lead, tungsten, and/or various other polymers. The lead plates may be formed of varying wall thicknesses, including, for example, ⅜ inches. In some embodiments, the loading system may be an extendable tray that selectively retracts and/or pivots back into place for use with the delivery device 500. The sled of the loading system may include a trough along a portion of the loading system where the vial assembly 580 is stored such that the trough receives and maintains any spills and/or leaks of fluid media from the vial body 589.

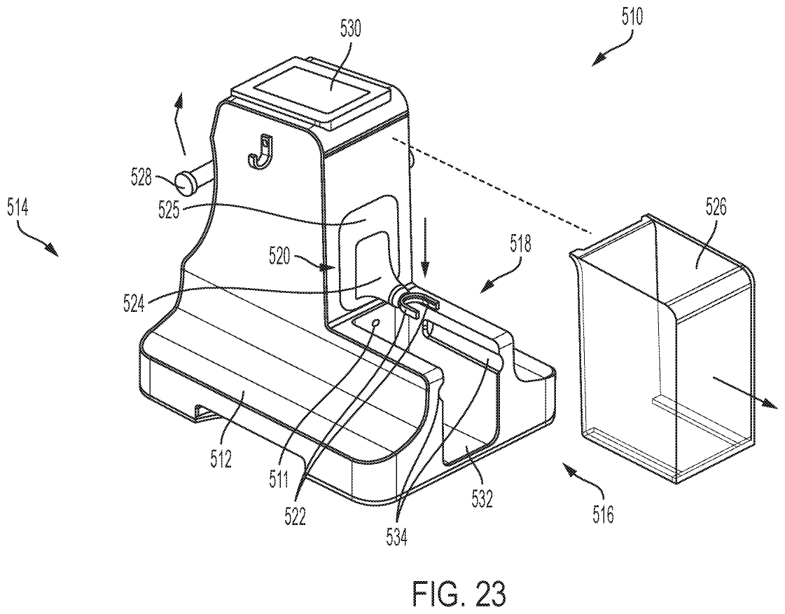

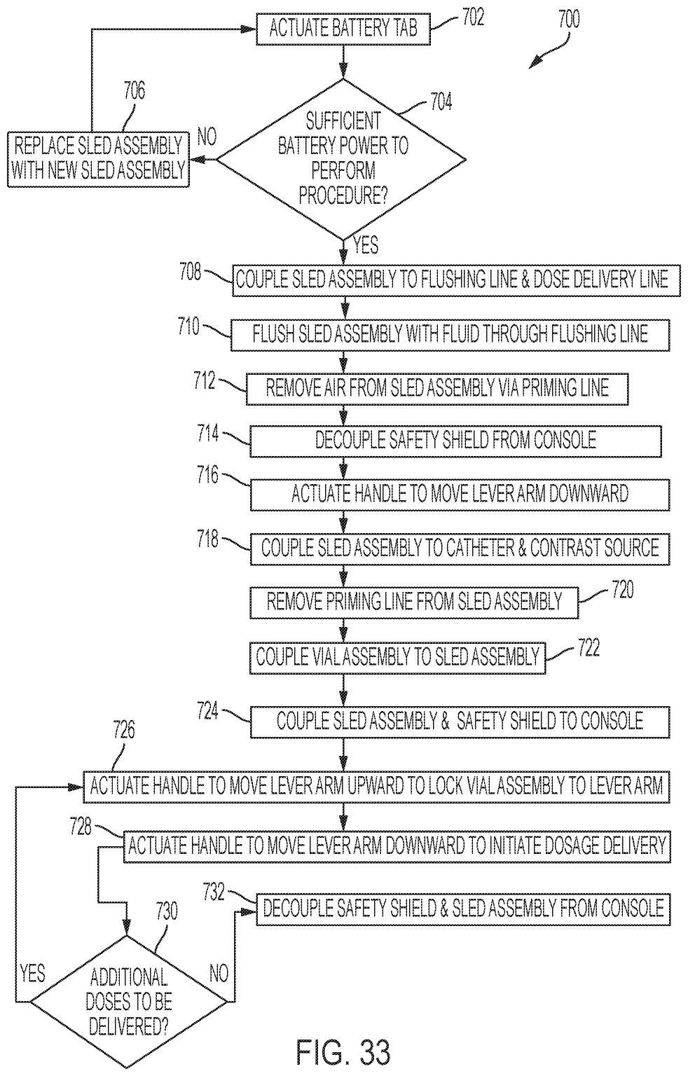

Referring now to FIGS. 23-32 in conjunction with the flow diagram of FIG. 33, an exemplary method 700 of operating the delivery device 500 is schematically depicted. The depiction of FIGS. 23-33 and accompanying description below is not meant to limit the subject matter described herein or represent an exact description of how a fluid media may be delivered using the delivery device 500, but instead is meant to provide a simple schematic overview to illustrate a general administration of a radioactive media from the delivery device 500 described herein.

At step 702 of FIG. 33, the removable tab 576 of the removable battery pack 570 is actuated to determine a quantity of power contained within the battery 572 of the removable battery pack 570. In particular, the removable tab 576 is removed from the removable battery pack 570 and a feedback output is generated identifying a status of the battery 572 of the removable battery pack 570. At step 706, an operator of the delivery device 500 determines whether the battery 572 of the removable battery pack 570 contains a sufficient amount of power to perform the procedure by observing the feedback output generated by the removable battery pack 570. In the present example, the removable battery pack 570 includes an LED status indicator 578 (see FIG. 24) that displays a green light when the battery 572 contains sufficient amount of power to perform a procedure and a red light when the battery 572 contains an insufficient amount of power to perform a procedure.

In response to determining that the battery 572 contains an insufficient amount of power at step 704, an operator replaces the sled assembly 540 with a new sled assembly 540 for use with the console assembly 510 to perform the procedure with at step 706. Alternatively, in other embodiments an operator may decouple the removable battery pack 570 from the sled assembly 540 and attach a new removable battery pack 570 to the original sled assembly 540, rather than replacing the sled assembly 540 entirely. In either instance, the exemplary method 700 returns to step 702 where the removable tab 576 of the new removable battery pack 570 is actuated to determine whether a sufficient amount of power exists in the battery 572 to perform the procedure.

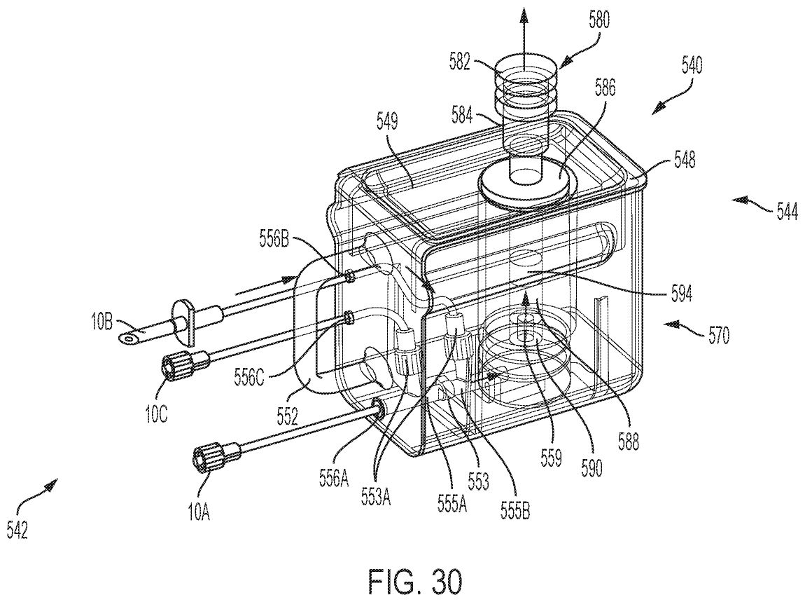

Referring now to FIG. 30, in response to determining that the battery 572 contains a sufficient amount of power at step 702, one or more delivery lines are coupled to the sled assembly 540 via the one or more ports 556 at step 708. In particular, a dose delivery line 10A is coupled to the sled assembly 540 at a delivery port 556A, a contrast line 10B is coupled to the sled assembly 540 at a contrast port 556B, and a flushing line 10C is coupled to the sled assembly 540 at a flushing port 556C. An opposing end of the dose delivery line 10A is initially coupled to a fluid reservoir, such as, for example, a collection bowl. As will be described in greater detail herein, the dose delivery line 10A may be subsequently coupled to an external device, such as a catheter, once the sled assembly 540 has been effectively primed by a fluid medium via the contrast line 10B. An opposing end of the flushing line 10C is coupled to an external device, such as, for example, a syringe. With both the dose delivery line 10A and the flushing line 10C coupled to the sled assembly 540, the sled assembly 540 is flushed with a fluid medium (e.g., saline) from the syringe coupled to the flushing line 10C at step 710. In this instance, the fluid medium is injected through the flushing line 10C, into the distal manifold 555A of the sled assembly 540, and out of the sled assembly 540 through the dose delivery line 10A. Accordingly, the fluid medium is ultimately received at the collection bowl and disposed thereat by the dose delivery line 10A. It should be understood that in other embodiments where the console assembly 510 and/or the sled assembly 540 are electrically coupled to an external power source in lieu of the removable battery pack 570 described above, the corresponding steps 702, 704, 706 of the exemplary method 700 described above may be substituted and/or omitted entirely without departing from the scope of the present disclosure.

With the distal manifold 555A of the sled assembly 540 separated from the proximal manifold 555B by the one-way valve 553 disposed therebetween, the fluid medium flushed through the distal manifold 555A from the syringe (via the flushing port 556C) is prevented from passing through the proximal manifold 555B and the needle 559 coupled thereto. Rather, the fluid medium injected from the syringe and through the flushing line 10C is received at the flushing port 556C, passed through the distal manifold 555A in fluid communication with the flushing port 556C, and redirected by the one-way valve 553 towards the dose delivery port 556A that is coupled to the dose delivery line 10A. In this instance, the dose delivery line 10A receives and transfers the fluid medium to the collection bowl coupled thereto, such that the fluid medium is not directed beyond the one-way valve 553 and into the proximal manifold 555B that is in fluid communication with the needle 559. It should be understood that step 710 may be repeated as necessary to effectively flush the sled assembly 540 and the dose delivery line 10A coupled thereto.

Referring back to FIG. 24 at step 712, the contrast line 10B is coupled to the sled assembly 540 at a contrast port 556B. An opposing end of the contrast line 10B is coupled to a fluid medium supply, such as, for example, a bag secured to the console assembly 510 via the attachment device 538. In the present example, the bag is a saline bag such that the fluid medium stored therein is saline. In this instance, with the sled assembly 540 including the priming assembly 560 positioned within the vial chamber 558 and the needle end 568 in fluid communication with the needle 559, a syringe is fluidly coupled to the priming line 562 of the priming assembly 560 and a plunger of the syringe is drawn back to pull saline through the contrast line 10B, the contrast port 556B, the sled assembly 540, the priming line 562 and into the syringe from the saline bag. The plunger of the syringe is thereafter pushed inwards to transfer the extracted saline back through the priming line 562, the central body 564, the elongated shaft 566, and the needle end 568 of the priming assembly 560 such that the saline is received into the needle 559 of the sled assembly 540. Accordingly, the manifolds 555A, 555B of the sled assembly 540 are effectively primed with the saline from the syringe as the needle 559 that received the saline from the priming assembly 560 is in fluid communication with the manifolds 555A, 555B. With the manifolds 555A, 555B in further fluid communication with the dose delivery line 10A via the delivery port 556A, the saline is effectively distributed to the collection bowl coupled thereto. It should be understood that step 712 may be repeated as necessary to remove all air from the sled assembly 540 and the collection line coupled thereto.

Referring now to FIG. 23 and at step 714, the safety shield 526 of the console assembly 510 is decoupled from the base 512 such that the vial containment region 518 is exposed. With the vial engagement mechanism 520 positioned within the vial containment region 518, and the safety shield 526 removed from the base 512 of the console assembly 510, the vial engagement mechanism 520 is readily accessible to an operator of the delivery device 500. At step 716, the handle 528 of the console assembly 510 is actuated to thereby move the vial engagement mechanism 520 with the vial containment region 518. More specifically, the handle 528 is translated and/or pivoted upward relative to the base 512 to thereby translate the pair of lever arms 522 and the neck 524 of the vial engagement mechanism 520 downward relative to the base 512, such that the vial engagement mechanism 520 is positioned proximate to the sled cavity 532.

Referring now to FIG. 30 and at step 718, the sled assembly 540 is coupled to one or more external devices via the one or more ports 556. In particular, the sled assembly 540 is fluidly coupled to a catheter (e.g., microcatheter) via the dose delivery line 10A that is coupled to the delivery port 556A of the sled assembly 540. In this instance, the catheter is in fluid communication with the sled assembly 540 via the dose delivery line 10A. Further at step 718, the sled assembly 540 is fluidly coupled to a contrast source, such as, for example, a saline bag secured to the console assembly 510 via the attachment device 538 (See FIG. 13). The sled assembly 540 is in fluid communication with the saline bag via a contrast line 10B coupled to the contrast port 556B of the sled assembly 540. In this instance, the saline bag is in fluid communication with the sled assembly 540 via the contrast line 10B secured to the contrast port 556B.

The contrast port 556B is in fluid communication with the proximal manifold 555B while the delivery port 556A is in fluid communication with the distal manifold 555A. As will be described in greater detail herein, saline from the saline bag may be withdrawn through the needle 559 of the sled assembly 540 and into the vial body 589 of the vial assembly 580 as the contrast port 556B is coupled to the proximal manifold 555B, rather than the distal manifold 555A which is separated from the proximal manifold 555B by the one-way check valve 553 disposed therebetween.

Referring now to FIG. 24 and step 720, the priming assembly 560 is removed from the sled assembly 540 by grasping the handle 563 and withdrawing the priming assembly 560 outwardly from the vial chamber 558. As the handle 563 is pulled from the sled assembly 540 through the aperture formed by the locking system 550, the needle end 568 of the priming assembly 560 is decoupled from the needle 559 of the sled assembly 540. In some embodiments, a feedback is generated (e.g., mechanical, tactile, etc.) indicating a detachment of the needle end 568 from the needle 559 such that an operator receives an indication of the disconnection.

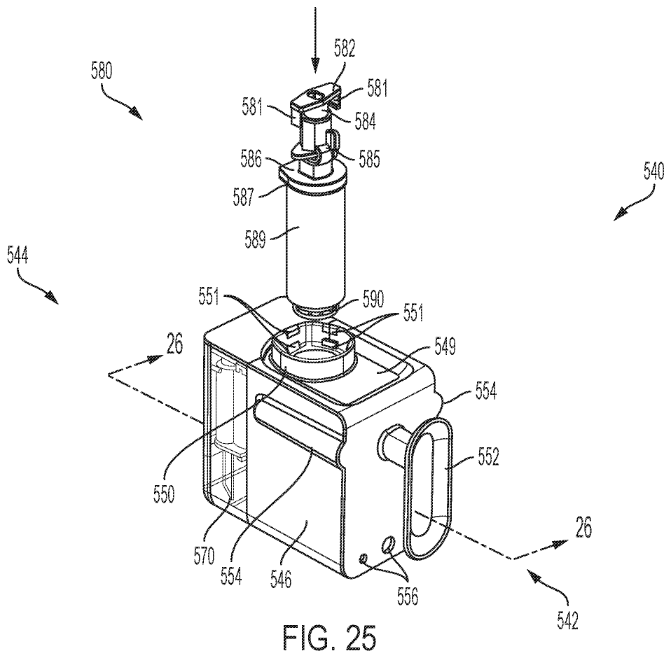

Referring now to FIG. 25 at step 722, the vial assembly 580 is slidably inserted into the sled assembly 540. In particular, the vial assembly 580 is removed from the sterile container assembly 600 in which the vial assembly 580 is initially stored in. The vial assembly 580 is removed from the sterile container assembly 600 by separating the housings 602, 612 of the sterile container assembly 600 in response to a decoupling of the corresponding mating systems 608, 618 of the housings 602, 612. With the sterile container assembly 600 containing the gasket seal 610 and the liquid seals along the closed ends 604, 614 of both housings 602, 612, the sterile container assembly 600 effectively maintains the radioactive media stored within the vial assembly 580 during a storage and transport of the vial assembly 580 for use in the procedure. It should be understood that in some embodiments the sterile container assembly 600 housing the vial assembly 580 therein may be stored within a lead pot until use of the vial assembly 580 is required. The cap 590 of the vial assembly 580 is inserted through the aperture formed by the locking system 550 at the top surface 548 of the sled assembly 540 and the vial assembly 580 is gradually inserted therethrough until the locking feature 586 contacts the locking system 550.

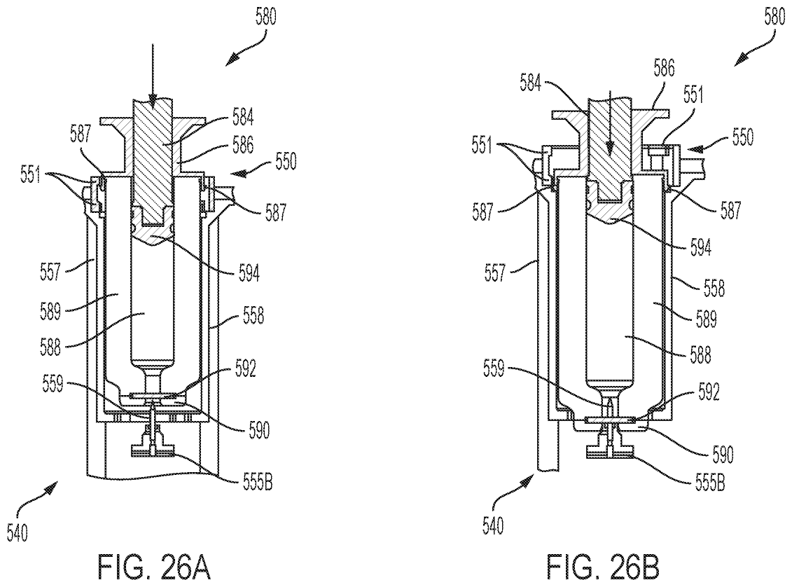

Referring now to FIG. 26A, the vial assembly 580 is shown disposed within the vial assembly 580, and in particular the vial body 589 is inserted within the vial chamber 558 with the cap 590 positioned proximate to the needle 559. In this instance, the lateral edge 587 of the locking feature 586 encounters a first row of the annular array of projections 551 of the locking system 550. Continued advancement of the vial assembly 580 into the sled assembly 540 causes the annular array of projections 551 positioned along the first row to flex outwardly in response to an application of force generated thereon by the lateral edge 587. In other words, the lateral edge 587 of the locking feature 586 presses outwardly against the annular array of projections 551 in response to the vial assembly 580 being received within the vial chamber 558.

As the annular array of projections 551 of the locking system 550 flex outwardly relative to the lateral edge 587 disposed therein, a continued translation of the vial assembly 580 into the vial chamber 558 causes the lateral edge 587 of the locking feature 586 to advance beyond a first row of the annular array of projections 551 such that the applied-force thereon from the lateral edge 587 is removed. In this instance, the annular array of projections 551 along the first row are permitted to flex inwardly and return to a default position with the lateral edge 587 positioned underneath the first row of projections 551. In some embodiments, a feedback is generated (e.g., an audible click) by the annular array of projections 551 when the lateral edge 587 is extended therethrough to thereby indicate to an operator that the vial assembly 580 is engaged with the locking system 550. Accordingly, with the first row of projections 551 positioned over the lateral edge 587 of the locking feature 586, the locking system 550 effectively inhibits a withdrawal of the vial assembly 580 from the vial chamber 558 of the sled assembly 540 due to an impediment formed by the first row of projections 551. In this instance, the needle 559 is positioned against and/or received through the cap 590 but is not in contact with the septum 592.

Referring now to FIG. 26B, a continued translation of the vial assembly 580 into the vial chamber 558 of the sled assembly 540 provides for a subsequent engagement between the lateral edge 587 of the locking feature 586 and the locking system 550. In particular, the lateral edge 587 encounters a second row of the annular array of projections 551 of the locking system 550. Continued advancement of the vial assembly 580 into the sled assembly 540 causes the projections 551 positioned along the second row to flex outwardly in response to an application of force generated thereon by the lateral edge 587. As the lateral edge 587 advances past the projections 551, the lateral edge 587 presses outwardly against the projections 551 until the lateral edge 587 of the locking feature 586 advances beyond the second row of projections 551.

In this instance, the applied-force from the lateral edge 587 is removed and the annular array of projections 551 along the second row are permitted to flex inwardly and return to a default position with the lateral edge 587 positioned underneath the second row of projections 551. Accordingly, with the second row of projections 551 positioned over the lateral edge 587 of the locking feature 586, the locking system 550 effectively inhibits a withdrawal of the vial assembly 580 from the vial chamber 558 of the sled assembly 540 due to an impediment formed by the second row of projections 551. In this instance, the needle 559 is positioned against and received through the cap 590 and the septum 592. More particularly, the needle 559 punctures the septum 592 of the vial assembly 580 such that the sled assembly 540 is in fluid communication with the vial body 589 of the vial assembly 580 through the needle 559.

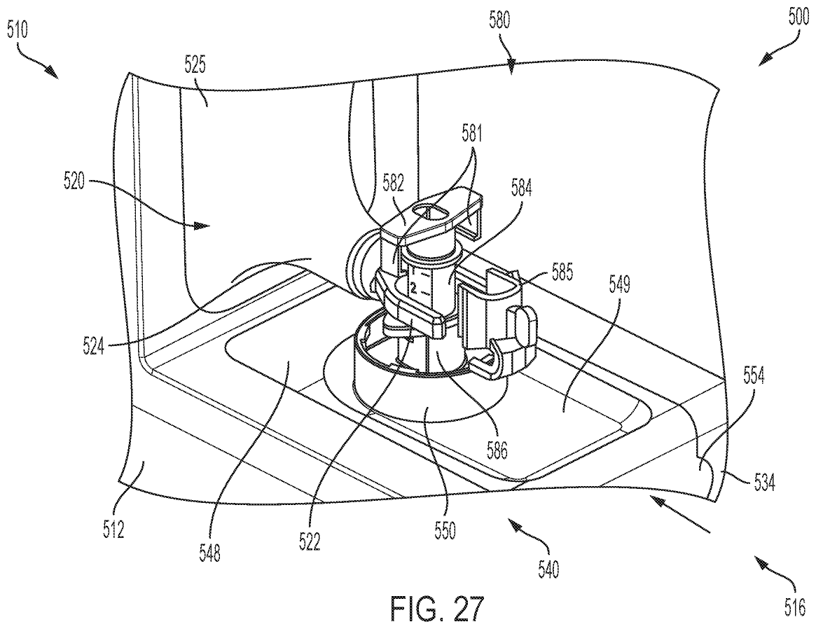

Referring now to FIG. 27 and at step 724, with the vial assembly 580 securely coupled to the sled assembly 540, the sled assembly 540 is coupled to the console assembly 510 by translating the proximal end 542 of the sled assembly 540 toward and into the distal end 516 of the console assembly 510. In particular, the proximal end 542 of the sled assembly 540 is directed into the sled cavity 532 of the console assembly 510 by aligning the alignment ribs 554 of the sled assembly 540 with the alignment features 534 of the console assembly 510. Once the distal end 544 and the proximal end 542 of the sled assembly 540 are fully seated within the sled cavity 532 of the console assembly 510, the electrical contacts 574 of the removable battery pack 570 interact with corresponding electrical contacts 511 of the console assembly 510 (See FIG. 23). In this instance, power from the battery 572 is transmitted to the console assembly 510 via the electrical contacts 574, thereby activating the console assembly 510 of the delivery device 500. In this instance, the interface display 530 of the console assembly 510 is activated to display pertinent, real-time information relating to the delivery device 500 during a procedure.

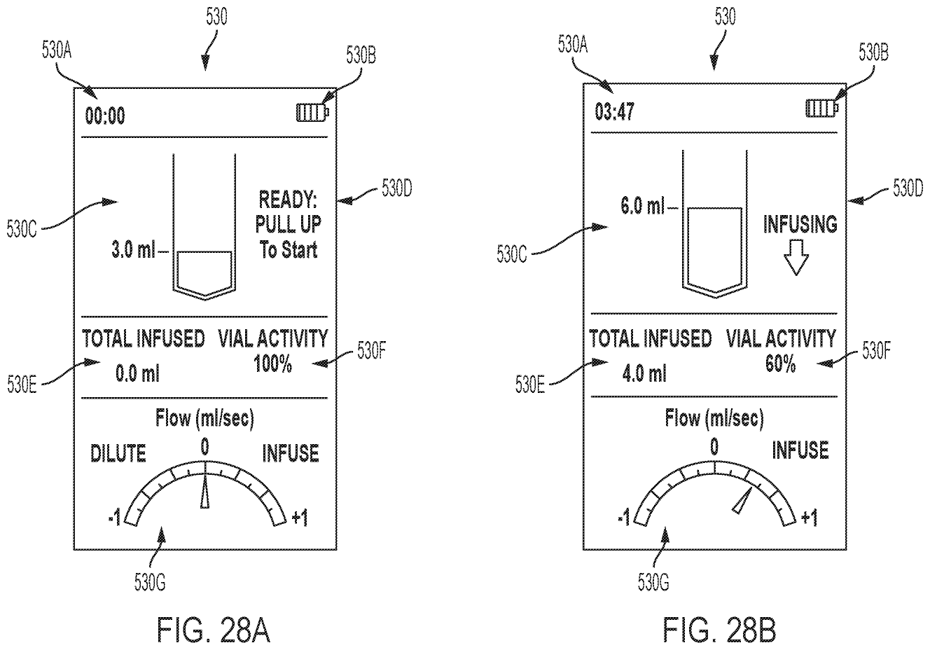

Referring to FIG. 28A, a schematic illustration of the interface display 530 is shown, where the interface display 530 of the console assembly 510 provides numerous data relating to the delivery device 500. As merely an illustrative example, the interface display 530 of the present examples displays data relating to at least a total duration 530A of a dose delivery; a lifespan 530B of the battery 572; a volume 530C of fluid media stored in the vial assembly 580; a current status 530D of the delivery device 500; a total volume 530E of fluid media infused by the delivery device 500; a radioactive percentage 530F of the fluid media stored within the vial assembly 580; and/or a volumetric infusion/dilution flow rate 530G of fluid media being delivered and/or drawn by the delivery device 500.

At step 724, with the sled assembly 540 having been coupled to the console assembly 510, the interface display 530 indicates a commencement of a procedure with the delivery device 500 such that the data displayed thereon is indicative of such. As a use of the delivery device 500 progresses the data displayed along the interface display 530 may progressively update to reflect a current condition and characteristics of the delivery device 500. It should be understood that the various information items 530A-530G shown and described herein are merely for illustrative purposes such that additional and/or fewer data may be detected, monitored, and displayed by the delivery device 500 at the interface display 530.

Referring back to FIG. 27, with the distal end 544 of the sled assembly 540 fully seated within the sled cavity 532 and the vial engagement mechanism 520 translated to a lower position at step 716, the pair of lever arms 522 engage the safety tab 585 of the vial assembly 580 thereby decoupling the safety tab 585 from the plunger 584. In other words, as the sled assembly 540 is translated into the sled cavity 532 in response to a force applied along the handle 552 at the proximal end 542, a position of the lever arms 522 of the vial engagement mechanism 520 are aligned with and encounter the safety tab 585 of the vial assembly 580. Accordingly, a continued translation of the sled assembly 540 into the sled cavity 532 provides for a disengagement of the safety tab 585 from the plunger 584 by the pair of lever arms 522. In this instance, the plunger 584 of the vial assembly 580 is uninhibited from translating into and/or out of the internal chamber 588 of the vial body 589 in response to an actuation of the vial engagement mechanism 520 coupled thereto.

Additionally at step 724, the safety shield 526 is coupled onto the base 512 of the console assembly 510 and over the vial containment region 518. In this instance, with the safety shield 526 attached to the base 512 of the console assembly 510 over the vial containment region 518, the safety shield 526 encloses the vial engagement mechanism 520, the vial assembly 580, and the sled assembly 540 within the vial containment region 518. Accordingly, during a procedure with the delivery device 500, the safety shield 526 maintains the one or more components of the delivery device 500 described herein enclosed within the vial containment region 518 to thereby shield an operator and/or patient from one or more fluid medias (e.g., radioembolizing microspheres) transferred between the console assembly 510, the sled assembly 540, and/or the vial assembly 580.

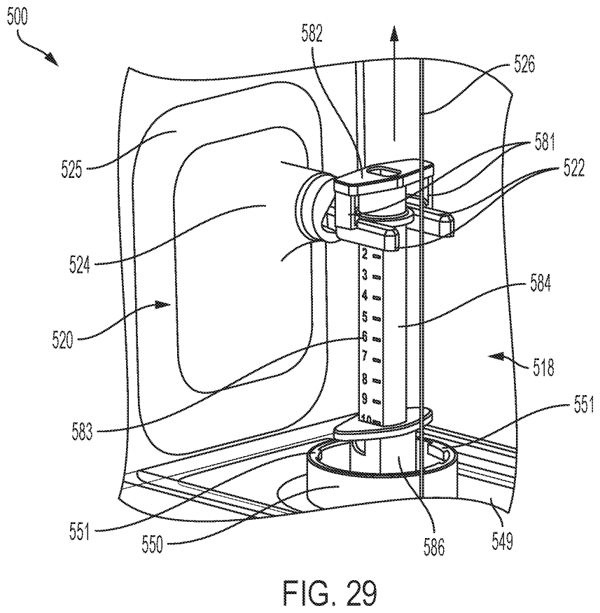

Referring now to FIG. 29 and at step 726, the handle 528 of the console assembly 510 is actuated (e.g., translated relative downward) to thereby move (e.g., linearly translate) the vial engagement mechanism 520 within the vial containment region 518 distally away from the sled cavity 532 and the sled assembly 540 received therein. In this instance, with the pair of lever arms 522 of the vial engagement mechanism 520 positioned about the plunger 584 of the vial assembly 580, translation of the neck 524 and the pair of lever arms 522 causes the pair of lever arms 522 to engage the engagement head 582, and in particular a bottom end of the pair of arms 581. With a removal of the safety tab 585, the plunger 584 is operable to translate upward and out of the vial body 589 of the vial assembly 580 in response to a translation of the vial engagement mechanism 520. Accordingly, the plunger 584 translates upward simultaneous with the translation of the vial engagement mechanism 520, due to the pair of arms 581 of the engagement head 582 being pulled upwardly by the pair of lever arms 522, in response to an actuation of the handle 528 at step 726.

In this instance, the pair of lever arms 522 of the vial engagement mechanism 520 is not securely coupled to the pair of arms 581 of the engagement head 582. Rather, the pair of lever arms 522 are merely positioned beneath the pair of arms 581 such that translation of the neck 524 of the vial engagement mechanism 520 causes the pair of lever arms 522 to abut against and pull the pair of arms 581 upward. It should be understood that the annular array of projections 551 of the locking system 550 inhibits a movement and/or an upward translation of the vial assembly 580, and in particular the vial body 589, from the vial chamber 558 of the sled assembly 540 as the vial engagement mechanism 520 pulls the plunger 584 of the vial assembly 580 relatively upward within the vial containment region 518. Additionally, it should further be understood that the alignment features 534 of the console assembly 510 inhibit a movement and/or upward translation of the sled assembly 540 from the sled cavity 532 of the console assembly 510 as the vial engagement mechanism 520 pulls the vial assembly 580 stored within the sled assembly 540 relatively upward within the vial containment region 518.

Still referring to FIG. 29, continued actuation of the handle 528 of the console assembly 510 provides for a continued translation of the vial engagement mechanism 520, and the plunger 584 as a result, until the annular washer 596 encounters the locking feature 586 (See FIG. 20). In this instance, the annular washer 596 inhibits the plunger 584 from translating further relative to the vial body 589 despite a continued actuation of the handle 528 of the console assembly 510. With the annular washer 596 of the vial assembly 580 abutting against the locking feature 586 and thereby inhibiting the plunger 584 from further translating out of the internal chamber 588 of the vial body 589 (and the aperture formed by the locking system 550), continued actuation of the handle 528 causes the pair of arms 581 of the engagement head 582 to flex outwardly relative to the plunger 584 due to an upward force applied thereto by the pair of lever arms 522 in response to the vial engagement mechanism 520 translating upward and the plunger 584 being inhibited from moving further.

In other words, with the pair of lever arms 522 pressed against the pair of arms 581 of the engagement head 582, continued translation of the neck 524 of the vial engagement mechanism 520 causes the pair of lever arms 522 to translate upward thereby applying a force against the pair of arms 581 of the engagement head 582. With the engagement head 582 integrally formed with the plunger 584 and the plunger 584 inhibited from translating further relative to the vial body 589 due to an impediment formed between the annular washer 596 and the locking feature 586, the pair of arms 581 of the engagement head 582 are flexibly deformed to expand outwardly to accommodate an upward translation of the pair of lever arms 522. As a result, the pair of lever arms 522 of the vial engagement mechanism 520 are securely coupled to the pair of arms 581 of the engagement head 582 via a snap-fit engagement, thereby locking the vial engagement mechanism 520 to the vial assembly 580.

Referring now to FIG. 30, as the vial engagement mechanism 520 and the plunger 584 are simultaneously translated within the vial containment region 518, a negative pressure is generated within the internal chamber 588 of the vial body 589 due to a retraction of the stopper 594. In this instance, with the saline bag coupled to the sled assembly 540 via the contrast line 10B and the contrast port 556B, saline from the saline bag is pulled into the internal chamber 588 of the vial body 589 through the proximal manifold 555B and the needle 559. Accordingly, with the vial body 589 being preloaded with a radioactive fluid media (e.g., radioembolizing microspheres), the saline is effectively mixed with the radioactive fluid media within the vial body 589 as the plunger 584 is retracted from the internal chamber 588 and the negative pressure is generated through the delivery device 500.

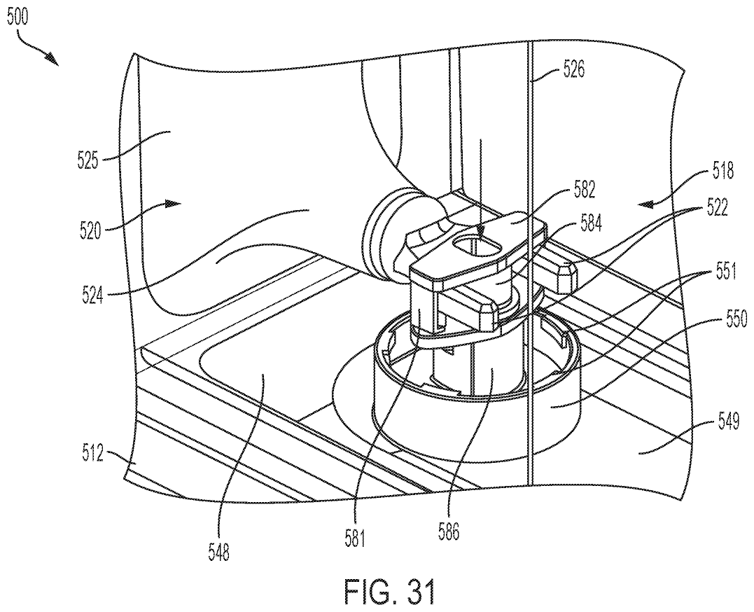

Referring now to FIG. 31 and at step 728, actuation of the handle 528 in an opposite direction (e.g., translated and/or pivoted downward relative to the base 512) provides for a simultaneous movement (e.g., linear translation) of the vial engagement mechanism 520. In this instance, the neck 524 translates downward toward the sled cavity 532 thereby causing the plunger 584 to translate into the vial body 589 due to a secured engagement between the pair of arms 581 of the engagement head 582 and the pair of lever arms 522 of the vial engagement mechanism 520. With the stopper 594 movably disposed within the vial body 589, translation of the plunger 584 causes a simultaneous translation of the stopper 594 through the vial body 589 thereby generating a positive pressure therein. As a result, a dose of the saline and radioactive fluid media mixture stored within the internal chamber 588 is transferred outward of the vial body 589 through the needle 559 and into the proximal manifold 555B. With the one-way check valve 553 configured to permit fluid communication from the proximal manifold 555B to the distal manifold 555A, the dose is delivered therethrough and into the dose delivery line 10A via the dose delivery port 556A.

Referring back to FIG. 30, the sled assembly 540 further includes one-way check valves 553A in-line with the contrast line 10B and the flushing line 10C. In particular, the one-way check valves 553A are configured to permit fluid communication from the contrast port 556B and the flushing port 556C into the manifolds 555A, 555B, and further configured to prevent fluid communication from the manifolds 555A, 555B to the contrast port 556B and the flushing port 556C. Accordingly, it should be understood that the dose delivered from the vial body 589 to the manifold 555A, 555B is incapable of being directed into the contrast line 10B or the flushing line 10C due to the one-way check valves 553A positioned therein. Thus, the dose is directed to the dose delivery port 556A and received at the catheter fluidly coupled thereto by the dose delivery line 10A. In other words, the one-way check valves 553A prevent a backflow of fluid into the sled assembly 540 and/or the vial assembly 580 coupled thereto.

Referring now to FIG. 33 at step 730, an operator determines whether delivery of additional doses from the delivery device 500 to the catheter is required during a procedure. In response to determining that additional doses for delivery are required at step 730, step 726 and 728 are repeated as necessary to effectively delivery a required volume of the dosage. An operator may monitor the interface display 530 of the console assembly 510 to review the various information presented thereon to determine whether additional dose deliveries are necessary at step 730. As described in greater detail above, the one or more sensors of the delivery device 500, and in particular at least the linear displacement sensor 531 and the radiation sensor 533 are configured to detect and measure various characteristics of the delivery device 500 and/or the media stored therein for display at the interface display 530.

Referring now to FIG. 28B, another schematic illustration of the interface display 530 is shown, where the interface display 530 of the console assembly 510 provides the various data relating to the delivery device 500 as described in greater detail above. In particular, at steps 726, 728 and 730, the interface display 530 continuously indicates a progressive status of the delivery device 500 during a procedure. As a use of the delivery device 500 progresses during steps 726, 728 and 730, the data displayed along the interface display 530 progressively updates to reflect a current condition and characteristic of the delivery device 500.

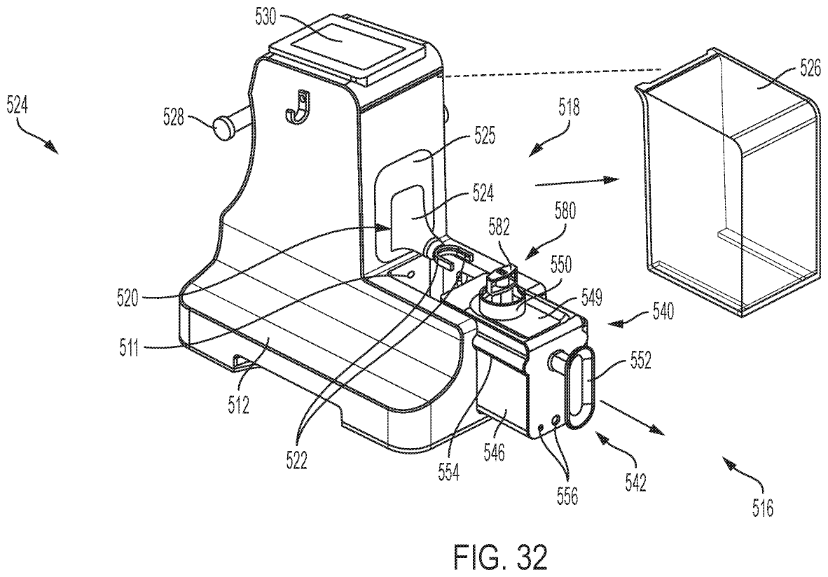

Referring to FIG. 32, in response to determining that additional doses for delivery are not required at step 730, the safety shield 526 is decoupled from the base 512 of the console assembly 510 to thereby expose the vial containment region 518 encapsulated therein at step 732. Additionally, the sled assembly 540 is decoupled from the sled cavity 532 of the console assembly 510 to thereby remove the sled assembly 540 from the vial containment region 518 at step 732. Upon separating the distal end 544 of the sled assembly 540 from base 512 of the console assembly 510, an engagement of the electrical contacts 574 of the removable battery pack 570 and the corresponding electrical contacts 511 of the console assembly 510 is terminated such that a power supply to the console assembly 510 is removed. Accordingly, the one or more components of the delivery device 500 requiring electrical power, such as, for example, the interface display 530, cease to be operable. In this instance, the sled assembly 540 and the vial assembly 580 are collectively discarded due to the fixed assembly of the locking feature 586 and the locking system 550. In other instances, the removable battery pack 570 is disengaged from the sled assembly 540 prior to discarding the sled assembly 540 and the vial assembly 580. In this instance, the removable battery pack 570 containing the battery 572 is discarded separate from the sled assembly 540.

VI. Motorized Delivery Device with Sled Assembly

As briefly noted above, in some embodiments the delivery device 500 may include a motorized system in lieu of the mechanical assembly 529 shown and described herein. For example, the handle 528 may be communicatively coupled to the vial engagement mechanism 520 via an electrical linkage with at least one motor coupled therebetween. In this embodiment, actuation of the handle 528 to draw in a fluid media from the vial assembly 580 and to subsequently deliver the fluid media from the delivery device 500 is electrically-driven at a predetermined flow rate by computer readable and executable instructions executed by a processor. In other embodiments, the handle 528 is communicatively coupled to the vial engagement mechanism 520 via an electrical linkage with at least one motor coupled to each of the handle 528 and the vial engagement mechanism 520. In this embodiment, actuation of the handle 528 to draw in the fluid media may be mechanically-driven as shown and described above, where the handle 528 is translated relatively downward by an operator to translate the vial engagement mechanism 520 linearly upward relative to the vial containment region 518. It should be understood that the processor and memory storing the computer readable and executable instructions may be located at the delivery device 500, a remote device, and/or both.

In either embodiment, a manual actuation of the handle 528 to infuse a dose of fluid media stored within the vial body 589 of the vial assembly 580 initiates a driving motor communicatively coupled to the handle 528, where the driving motor is configured to generate a resistant force against the handle 528 proportionate and counter to the manual force applied thereto by an operator. In this instance, a haptic feedback is generated by the motor at the handle 528 in response to a physical manipulation of the handle 528 during a delivery of media from the delivery device 500. A degree of resistive force generated by the motor at the handle 528 corresponds to a predetermined volumetric infusion flow rate preprogrammed in and/or determined by the computer readable and executable instructions executed by the processor. Accordingly, a manual manipulation of the handle 528 during an infusion process of the delivery device 500, to a degree that alters a current infusion flow rate from the predetermined infusion flow rate, causes the motor to generate a resistance against the handle 528.

It should be understood that the motor communicatively coupled to the handle 528 inhibits and does not prevent manual actuation of the handle 528, such that a degree of resistive force and haptic feedback generated at the handle 528 corresponds to, and increases with, a variance of a current infusion flow rate from a predetermined infusion flow rate. In the present example, continued manual actuation of the handle 528 to a degree that increases a variance between a current infusion flow rate and a predetermined infusion flow rate causes the motor communicatively coupled to the handle 528 to progressively generate an increased resistive force thereto, thereby providing a greater haptic feedback for an operator indicative of the increased threshold. With another motor coupled to the vial engagement mechanism 520, it should be understood that the driving motor coupled to the handle 528 is in communication with the motor coupled to the vial engagement mechanism 520 such that a manual actuation at the handle 528 is transmitted to the vial engagement mechanism 520. In this instance, an input by an operator at the handle 528 that overcomes the resistive force applied thereto is proportionally applied a linear translation of the vial engagement mechanism 520.

In other embodiments, the computer readable and executable instructions executed by the processor include a maximum variance threshold such that a manual actuation of the handle 528 by an operator of the delivery device 500 at a degree that exceeds the maximum variance threshold is prevented. The delivery device 500 may include one or more sensors coupled to the handle 528, the plunger 584, the vial engagement mechanism 520, the manifold 555A, 555B, and/or other components of the delivery device 500 to detect and monitor various characteristics of the delivery device 500. For example, the one or more sensors may be configured to measure a manual force applied by an operator to the handle 528, a linear displacement of the vial engagement mechanism 520, a current infusion flow rate of the delivery device 500, a torque of the driving motor coupled to the handle 528 and/or the vial engagement mechanism 520, and/or the like. By way of example, the one or more driving motors may comprise, but are not limited to, a linear stage actuator. Additionally, the one or more sensors may comprise, for example, a current sensor, a torque sensor, a pressure sensor, a flow sensor, and/or the like. Although not shown, it should be understood that in other embodiments the handle 528 of the delivery device 500 may be remotely located from the console assembly 510 such that a motor communicatively coupled to the handle 528 is similarly remote relative to the console assembly 510.

In some embodiments, a manual actuation sensitivity of the handle 528 may be selectively programmed and adjusted prior to a use of the delivery device 500. For example, the compute readable and executable instructions executed by the processor may include various settings for correlating a relative degree of movement at the handle 528 to a linear displacement of the vial engagement mechanism 520. In this instance, a coarse and/or fine manipulation of the handle 528 may initiate varying torques at the driving motor communicatively coupled to the vial engagement mechanism 520 for translating the vial engagement mechanism 520 within the vial containment region 518. An operator of the delivery device 500 may identify a predetermined infusion flow rate, a current infusion flow rate, and/or other data and characteristics pertaining a resistive force generated by the one or more driving motors along the interface display 530 of the console assembly 510.

### VII. Dual-Component Plunger

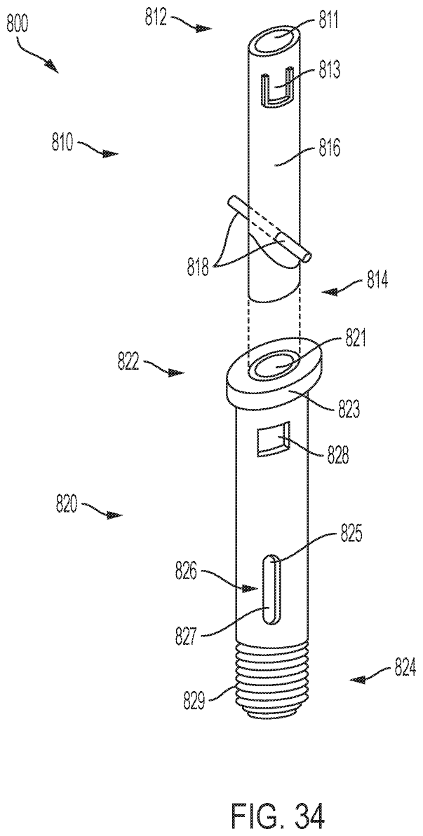

Referring now to FIG. 34, an alternative plunger assembly 800 is depicted. In the example shown and described herein, it should be understood that the plunger assembly 800 is configured and operable just like the plunger 584 described above except for the differences explicitly noted herein. Accordingly, the plunger assembly 800 of the present example may be readily incorporated into the vial assembly 580 described above. It should further be understood that the plunger assembly 800, in many respects, functions substantially similar to the plunger 584 described above such that a version of the vial assembly 580 that is equipped with the plunger assembly 800 of the present example may be configured and operable similar to the vial assembly 580 described above with the plunger 584 except for the differences described below.

The plunger assembly 800 comprises an inner member 810 and an outer member 820, with the outer member 820 sized and shaped to slidably receive the inner member 810 therethrough. In particular, the inner member 810 comprises a top end 812 and a bottom end 814 defining an elongated body 816 extending therebetween such that the ends 812, 814 define a longitudinal length of the elongated body 816. The top end 812 includes a top aperture 811 extending therethrough. It should be understood that the elongated body 816 defines a lumen extending through the inner member 810 from the top end 812 to the bottom end 814 such that the top aperture 811 is in communication with said lumen of the elongated body 816. In the present example, the elongated body 816 of the inner member 810 is cylindrically-shaped similar to a shape of the vial body 589 in which the plunger assembly 800 is slidably received in.

Still referring to FIG. 34, the inner member 810 includes a pair of flexible latches 813 positioned along the elongated body 816 adjacent to the top end 812. The pair of flexible latches 813 are resiliently biased to extend laterally outward from the elongated body 816. As will be described in greater detail herein, an application of a laterally inward force onto the pair of flexible latches 813 (i.e. toward the elongated body 816) causes the pair of flexible latches 813 to flexibly deform inwardly into the lumen defined by the elongated body 816. The inner member 810 further includes a pair of pins 818 extending laterally outward from the elongated body 816 adjacent to the bottom end 814. As will be described in greater detail herein, the pair of pins 818 are sized and shaped to be slidably received within a longitudinal slot 826 of the outer member 820.

The outer member 820 of the plunger assembly 800 comprises a top end 822 and a bottom end 824 defining an elongated body extending therebetween such that the ends 822, 824 define a longitudinal length of the elongated body. The top end 822 includes a top aperture 821 extending therethrough. It should be understood that the elongated body defines a lumen extending through the outer member 820 from the top end 822 to the bottom end 824 such that the top aperture 821 is in communication with said lumen of the outer member 820. The elongated body of the outer member 820 is shaped substantially similar to the inner member 810 such that the outer member 820 is sized and shaped to slidably receive the inner member 810 through the lumen defined by the elongated body. Accordingly, the elongated body of the outer member 820 is cylindrically-shaped similar to a shape of the vial body 589 in which the plunger assembly 800 is slidably received in.

Still referring to FIG. 34, the outer member 820 includes an engagement head 823 extending about the elongated body adjacent to the top aperture 821. In particular, the engagement head 823 extends about the elongated body at a lateral length such that the engagement head 823 includes a greater diameter than the elongated body. As will be described herein, a bottom surface of the engagement head 823 is sized such that the pair of lever arms 522 of the vial engagement mechanism 520 are received thereon in response to a vertical translation of the neck 524 and a corresponding linear displacement of the plunger assembly 800 relative to the vial body 589. Accordingly, it should be understood that the engagement head 823 of the outer member 820 and the pair of flexible latches 813 of the inner member 810 collectively serve as an equivalent structural substitute for the pair of arms 581 of the engagement head 582 of the plunger 584.

The outer member 820 further includes a pair of windows 828 disposed through the elongated body proximate to the top end 822 of the outer member 820. The pair of windows 828 extend into the lumen defined by the elongated body and are sized and shaped in accordance with a size and shape of the pair of flexible latches 813. As described in greater detail herein, the pair of windows 828 are configured to receive the pair of flexible latches 813 therethrough to securely fasten the inner member 810 to the outer member 820. As briefly noted above, the outer member 820 includes a pair of longitudinal slots 826 extending through the elongated body adjacent to the bottom end 824. In particular, the longitudinal slots 826 extend along opposing sides of the elongated body and are defined between an upper segment 825 and a lower segment 827. The longitudinal slots 826 are sized and shaped to slidably receive at least one of the pair of pins 818 of the inner member 810 therethrough. Additionally, the outer member 820 includes a stopper 829 that is substantially similar to the stopper 594 described above such that the stopper 829 is configured and operable just like the stopper 594.

Still referring to FIG. 34, in an exemplary mode of operation of the plunger assembly 800 with the vial assembly 580 described above, the inner member 810 is initially received with a lumen of the outer member 820 such that the top ends 812, 822 are flush with one another and the pair of flexible latches 813 are disposed within the lumen of the outer member 820. In particular, the pair of flexible latches 813 are positioned within the lumen of the outer member 820 between the top aperture 821 and the pair of windows 828. In this instance, an inner surface of the elongated body of the outer member 820 applies a laterally inward force against the pair of flexible latches 813 such that the pair of flexible latches 813 are deformed inwardly into a lumen of the inner member 810.

A resilient bias of the flexible latches 813 exerts an outward force against the laterally inward force generated by the elongated body such that a frictional interference is provided against the inner member 810 and the outer member 820 between the pair of flexible latches 813 and an inner surface of the elongated body. Accordingly, the inner member 810 is securely fixed within and relative to the outer member 820 prior to an actuation of the plunger assembly 800 in response to a linear translation of the vial engagement mechanism 520.

Still referring to FIG. 34, with the pair of flexible latches 813 disposed within a lumen of the elongated body and positioned between the top aperture 821 and the pair of windows 828 in a default position, the pair of pins 818 of the inner member 810 is received within the longitudinal slot 826. In particular, the pair of pins 818 are positioned along the upper segment 825 of the longitudinal slot 826 when the plunger assembly 800 is in a default position. With the plunger assembly 800 incorporated within the vial assembly 580 and the vial assembly 580 assembled with the sled assembly 540, a coupling of the sled assembly 540 with the console assembly 510 provides for an engagement of the pair of lever arms 522 with a bottom surface of the engagement head 823. Accordingly, an upward translation of the vial engagement mechanism 520 provides for an engagement with the bottom surface of the engagement head 823, thereby translating the plunger assembly 800 vertically upward relative to the vial body 589 of the vial assembly 580.

Still referring to FIG. 34, in the present example the vial body 589 and/or the locking feature 586 of the vial assembly 580 includes a retention feature that is sized and configured to engage the pair of pins 818 disposed within the vial body 589 upon a predetermined translation of the plunger assembly 800 relative to the vial body 589. In other words, the retention feature is positioned within the vial body 589 and/or the locking feature 586 at a location such that the retention feature engages the pair of pins 818 thereon after the plunger assembly 800 is vertically translated a predetermined distance relative to the vial body 589. It should be understood that a location of the retention feature, and the predetermined translation distance described above, is configured to correspond to a minimum threshold volume of fluid medium (e.g. saline) that is to be drawn into the internal chamber 588 in response to a linear displacement of the plunger assembly 800 therein.

Accordingly, locating the retention feature at the predetermined distance facilitates an extraction of the predetermined minimum volume threshold of fluid medium into the internal chamber 588 prior to a dose delivery by the delivery device. The predetermined minimum volume threshold may comprise various suitable quantities for creating a suitable mixture of the therapeutic particles and the fluid medium (e.g. saline) therein to ensure the resulting suspension fluid to be delivered is adequate for administration into a patient. For example, in some embodiments the predetermined minimum volume threshold may equal about 9 milliliters to 11 milliliters, and more specifically 10 milliliters.

Still referring to FIG. 34, once the plunger assembly 800 has translated the predetermined distance the pair of pins 818 arrive at, and engage, the retention feature thereby locking a vertical position of the pair of pins 818 thereat relative to the vial body 589. Continued actuation of the vial engagement mechanism 520 provides for a continued translation of the pair of lever arms 522 and the outer member 820 due to an engagement of a bottom surface of the engagement head 823 with the pair of lever arms 522. In this instance, the outer member 820 translates upward relative to the inner member 810, a vertical position of which is fixedly secured due to an engagement of the pair of pins 818 and the retention feature, such that the pair of pins 818 translate along the longitudinal slot 826 from the upper segment 825 to the lower segment 827. Additionally, the pair of flexible latches 813 of the inner member 810 translate within the lumen of the outer member 820 until arriving in alignment with the pair of windows 828. In this instance, the pair of flexible latches 813 extend outwardly and through the pair of windows 828 due to a termination of the inward lateral force generated against the pair of flexible latches 813 by an inner surface of the outer member 820. Accordingly, the pair of flexible latches 813 returns to a default configuration by extending laterally outward from a lumen of the elongated body 816 of the inner member 810 and through the pair of windows 828.

It should be understood that the pair of pins 818 of the inner member 810 are positioned at the lower segment 827 of the longitudinal slot 826 as the pair of flexible latches 813 are aligned with and received in the pair of windows 828. In this instance, the inner member 810 is fixedly secured to the outer member 820 such that a relative vertical position of the members 810, 820 is fixed. It should further be understood that the pair of flexible latches 813 protrude outwardly from the pair of windows 828 at a predetermined length that effectively increases a lateral width of the outer member 820 at a location along the pair of windows 828. In this instance, a downward translation of the neck 524 of the vial engagement mechanism 520 causes the pair of lever arms 522 to disengage from a bottom surface of the engagement head 823 and to engage the pair of flexible latches 813 positioned underneath such that the members 810, 820 of the plunger assembly 800 are effectively translated downward into the internal chamber 588 to deliver a dose therefrom.

### VIII. Dual-Winged Plunger

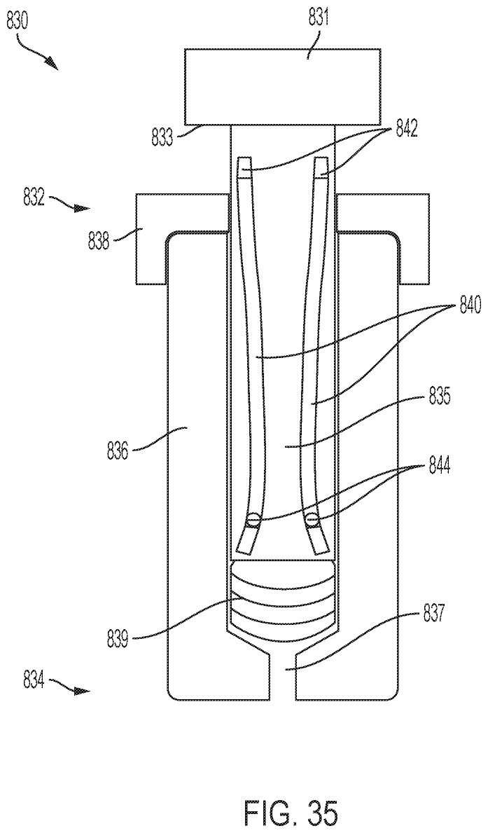

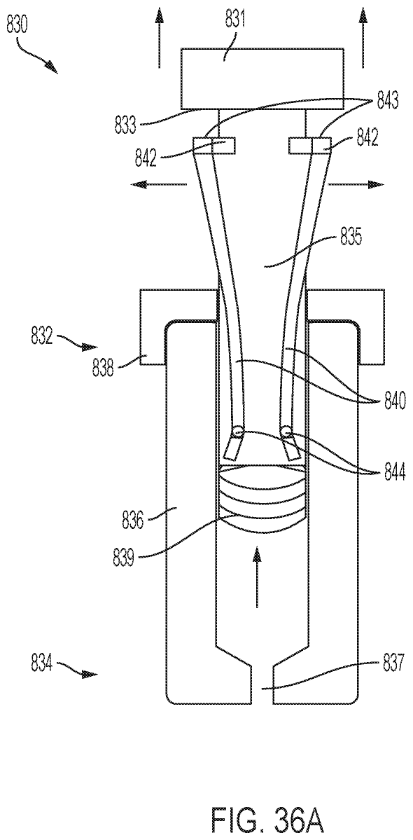

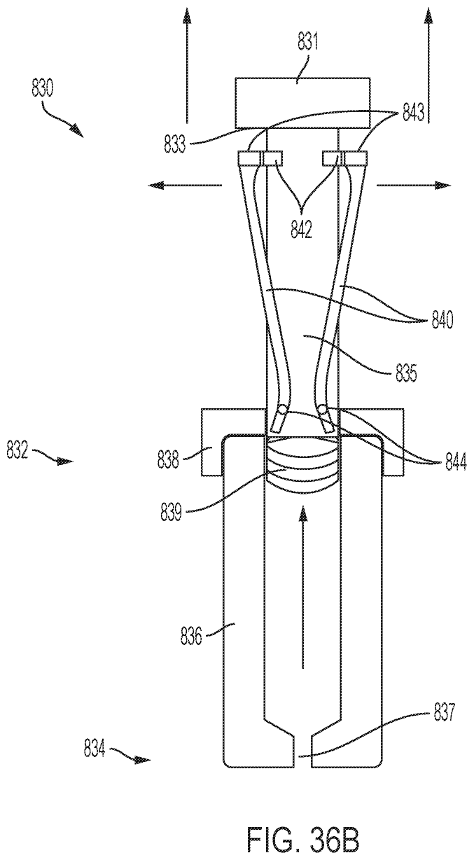

Referring now to FIGS. 35-36, an alternative vial assembly 830 is depicted. In the example shown and described herein, it should be understood that the vial assembly 830 is configured and operable just like the vial assembly 580 described above except for the differences explicitly noted herein. Accordingly, the vial assembly 830 of the present example may be readily incorporated into the sled assembly 540 described above. It should further be understood that the vial assembly 830, in many respects, functions substantially similar to the vial assembly 580 described above such that a version of the sled assembly 540 that is equipped with the vial assembly 830 of the present example may be configured and operable similar to the sled assembly 540 described above with the vial assembly 580 received therein except for the differences described below.

Specifically referring to FIG. 35, the vial assembly 830 comprises an engagement head 831, a locking feature 832, a plunger 835, a vial body 836 and a stopper 839. The engagement head 831 and the stopper 839 define a longitudinal length of the plunger 835. In other words, the engagement head 831 and the stopper 839 are positioned along opposing ends of the plunger 835. The elongated head 831 of the present example includes a bottom surface 833 facing proximately toward the locking feature 832, which includes a lateral edge 838 that extends about a top segment of the vial body 836. The stopper 839 is coupled to a bottom segment of the plunger 835 and is configured and operable similar to the stopper 594 of the vial assembly 580 described above. The cap 834 of the vial assembly 830 includes an aperture 837 at a terminal end of the vial body 836 that is sized and shaped to receive the needle 559 of the sled assembly 540 when the vial assembly 830 is coupled thereto. It should be understood that in some embodiments the aperture 837 may comprise one or more features therein for receiving the needle 559, such as, for example, an elastomer similar to the septum 592 of the vial assembly 580 described above.

The vial assembly 830 differs from the vial assembly 580 in that the plunger 835 includes a pair of flexible wings 840 coupled thereto. In particular, the pair of flexible wings 840 are movably coupled to an exterior surface of the plunger 835, and extend along a longitudinal length of the plunger 835. The pair of flexible wings 840 have a longitudinal length extending between a pivotable blade 842 and a rotatable coupler 844, each of which are coupled to the exterior surface of the plunger 835. In the present example, the pair of flexible wings 840 are shown in a default orientation with the pivotable blade 842 in a vertical configuration. With the pair of flexible wings 840 in a default orientation, a longitudinal length of the pair of flexible wings 840 are fully disposed within the vial body 836 of the vial assembly 830. As will be described in greater detail herein, the pivotable blade 842 of the pair of flexible wings 840 is configured to pivot laterally outward away from the plunger 835 of the vial assembly 830 in response to a vertical translation of the plunger 835 out of the vial body 836.

Referring now to FIG. 36A, in an exemplary mode of operation of the vial assembly 830, the vial engagement mechanism 520 engages the engagement head 831, and in particular the pair of lever arms 522 engage a bottom surface 833 of the engagement head 831. In this instance, actuation of the handle 528 provides an upward translation of the neck 524 which thereby causes the pair of lever arms 522 to translate vertically upward. With the pair of lever arms 522 engaged against the bottom surface 833 of the engagement head 831, the engagement head 831 and the plunger 835 are linearly displaced relative to the vial body 836 of the vial assembly 830. With an upward translation of the plunger 835, the pair of flexible wings 840 transition from a default orientation to a partially actuated position. In particular, the pair of flexible wings 840 rotate about the rotatable couplers 844 such that a longitudinal length of the pair of flexible wings 840 bow outward from the vial body 836. In other words, the pair of flexible wings 840 are configured to flexibly deform such that a longitudinal length of the pair of flexible wings 840 are curved outward from the vial body 836.

The pivotable blades 842 of the pair of flexible wings 840 pivot outwardly from the plunger 835 to thereby form an engagement surface 843 thereon. In other words, the pivotable blades 842 are configured to snap out and form the engagement surface 843 in response to a translation of the plunger 835 and a simultaneous rotation of the flexible wings 840 about the rotatable couplers 844. It should be understood that a length of the engagement surface 843 formed by the pivotable blades 842 is configured to engage the pair of lever arms 522, once the plunger 835 has translated a predetermined distance, with the predetermined distance corresponding to a minimum threshold volume of fluid medium (e.g. saline) that is to be drawn into the vial body 836 in response to a linear displacement of the plunger 835 therein.

Still referring to FIG. 36A, the plunger 835 is shown as translating a portion of the predetermined distance such that a length of the engagement surfaces 843 formed by the pivotable blades 842 of each of the flexible wings 840 is not operable to engage the pair of lever arms 522 during a downward translation of the neck 524 of the vial engagement mechanism 520. Rather, the engagement surfaces 843 are partially formed in this instance such that an opposite translation of the vial engagement mechanism 520 will cause the pair of lever arms 522 to linearly translate by the pair of pivotable blades 842 and thus not interact with and/or engage a corresponding feature of the vial assembly 830. In this instance, the plunger 835 is not pushed into the vial body 836, thereby not administering a dose for delivery.

It should be understood that extending the pivotable blades 842 out further, in response to a continued upward translation of the plunger 835, for engagement with the lever arms 522 facilitates an extraction of the predetermined minimum volume threshold of fluid medium into the vial body 836 prior to a dose delivery by the delivery device. The predetermined minimum volume threshold may comprise various suitable quantities for creating a suitable mixture of the therapeutic particles and the fluid medium (e.g. saline) therein to ensure the resulting suspension fluid to be delivered is adequate for administration into a patient. For example, in some embodiments the predetermined minimum volume threshold may equal about 9 milliliters to 11 milliliters, and more specifically 10 milliliters.

Referring now to FIG. 36B, once the plunger 835 has translated the predetermined distance the pair of pivotable blades 842 extend out from the plunger 835 at a greater length due to an increased deformation of the flexible wings 840. Continued actuation of the vial engagement mechanism 520 provides for a continued translation of the pair of lever arms 522 and the plunger 835 due to an engagement of the bottom surface 833 of the engagement head 831 with the pair of lever arms 522. In this instance, the plunger 835 translates upward relative to the vial body 836 such that the pair of flexible wings 840 bow out further from a longitudinal length of the plunger 835. As a result, the pair of pivotable blades 842 extend laterally outward thereby forming the engagement surfaces 843 at a greater length. In this instance, the pair of pivotable blades 842 extend outwardly in a horizontal configuration.

It should further be understood that the pair of engagement surfaces 843 protrude outwardly from the plunger 835 at a predetermined length that effectively increases a lateral width of the plunger 835 at a location along the pair of pivotable blades 842. In this instance, a downward translation of the neck 524 of the vial engagement mechanism 520 causes the pair of lever arms 522 to disengage from the bottom surface 833 of the engagement head 831 and to engage the engagement surface 843 of the pair of pivotable blades 842 positioned underneath such that the plunger 835 is effectively translated downward into the vial body 836 to deliver a dose therefrom.

### IX. Rotatable Plunger

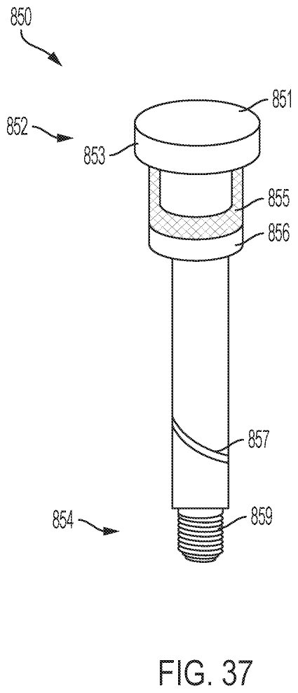

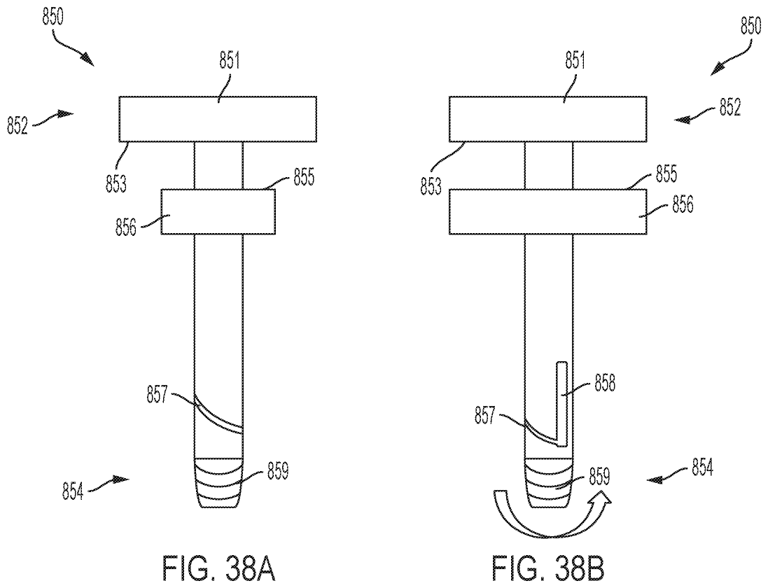

Referring now to FIGS. 37-38, an alternative plunger assembly 850 is depicted. In the example shown and described herein, it should be understood that the plunger assembly 850 is configured and operable just like the plunger 584 described above except for the differences explicitly noted herein. Accordingly, the plunger assembly 850 of the present example may be readily incorporated into the vial assembly 580 described above. It should further be understood that the plunger assembly 850, in many respects, functions substantially similar to the plunger 584 described above such that a version of the vial assembly 580 that is equipped with the plunger assembly 850 of the present example may be configured and operable similar to the vial assembly 580 described above with the plunger 584 except for the differences described below.

Referring specifically to FIG. 37, the plunger assembly 850 comprises a top end 852 and a bottom end 854 with a pair of engagement heads 851, 856 positioned along the top end 852. In particular, the plunger assembly 850 comprises an upper engagement head 851 and a lower engagement head 856, with a bottom surface 853 of the upper engagement head 851 positioned relatively above an top surface 855 of the lower engagement head 856. The plunger assembly 850 further includes a curved track 857 disposed along and extending about an exterior surface of the plunger assembly 850. With the plunger assembly 850 of the present example comprising a cylindrically-shaped profile, the curved track 857 is formed thereon such that the curved track 857 extends about the cylindrical shape of the plunger assembly 850. Although not shown, it should be understood that the curved track 857 is sized and shaped to slidably receive a pin from the vial body 589 and/or the locking feature 586 therein. In this instance, translation of the plunger assembly 850 with the pin received within the curved track 857 provides a translation of the plunger assembly 850 relative to the vial body 589 due a curved configuration of the curved track 857. As described in greater detail herein, the plunger assembly 850 further includes a linear track 858 disposed along and extending on an exterior surface of the plunger assembly 850 (see FIG. 38B), where the linear track 858 is parallel to a longitudinal length of the plunger assembly 850.

The plunger assembly 850 further includes a stopper 859 that is substantially similar to the stopper 594 of the plunger 584 described above. A size and shape of the upper engagement head 851 is distinct from a size and shape of the lower engagement head 856 such that the pair of engagement heads 851, 856 have varying profiles relative to one another. In the present example, the upper engagement head 851 comprises a circularly-shaped profile and the lower engagement head 856 comprises an oval and/or oblong-shaped profile. It should be understood that the engagement heads 851, 856 may comprise various other shapes and/or sizes than those shown and described herein without departing from a scope of the present disclosure. As will be described in greater detail herein, the shapes of the engagement heads 851, 856 are configured to vary relative to one another to facilitate a delivery of a predetermined minimum threshold of fluid medium from the vial body 589.

Referring now to FIG. 38A, the plunger assembly 850 is depicted in a first rotatable orientation relative to the vial body 589 of the vial assembly 580. For example, in the first orientation a width of the upper engagement head 851 is greater than a width of the lower engagement head 856 due to the relatively varying profiles of the engagement heads 851, 856, respectively. With the upper engagement head 851 comprising a circular shape in the present example, it should be understood that the upper engagement head 851 comprises a similar profile in the first orientation as in a plurality of other orientations, including, for example, a second rotatable orientation shown in FIG. 38B. In contrast, with the lower engagement head 856 comprising an oval and/or oblong shape, the lower engagement head 856 comprises varying profiles in a plurality of orientations. For example, in the first orientation a width of the lower engagement head 856 is less than a width of the lower engagement head 856 in a second orientation shown in FIG. 38B.

In an exemplary mode of operation of the plunger assembly 850, the vial engagement mechanism 520 is coupled to the plunger assembly 850 by receiving the pair of lever arms 522 between the upper engagement head 851 and the lower engagement head 856. In particular, the pair of lever arms 522 of the vial engagement mechanism 520 are slidably positioned between the pair of engagement head 851, 856 such that a vertical translation of the neck 524 of the vial engagement mechanism 520 causes an engagement of the bottom surface 853 of the upper engagement head 851 by the pair of lever arms 522 positioned underneath thereof. As briefly noted above, with the plunger assembly 850 received within the vial body 589 of the vial assembly 580, a pin extending from the vial body 589 and/or the locking feature 586 is slidably received within the curved track 857 of the plunger assembly 850.

Still referring to FIG. 38A, translation of the vial engagement mechanism 520, with the pair of lever arms 522 engaged against the bottom surface 853 of the upper engagement head 851, provides an upward translation of the plunger assembly 850 relative to the vial body 589. With a fixed pin of the vial body 589 slidably coupled to the plunger assembly 850 within the curved track 857, translation of the vial engagement mechanism 520 further provides a rotation of the plunger assembly 850 in a direction corresponding to a travel path of the fixed pin within the curved track 857. It should be understood that in an initial default position, the fixed pin of the vial assembly 580 is received along a top portion of the curved track 857. With the curved track 857 of the plunger assembly 850 extending relatively downward from the top portion toward the bottom end 854 and wrapping around the plunger assembly 850, the curved track 857 is configured to facilitate a rotation of the plunger assembly 850 along with a simultaneous upward translation relative to the vial body 589.

In this instance, the fixed pin travels through the curved track 857 from the top portion and toward a bottom portion of the curved track 857 adjacent to the bottom end 854. With the curved track 857 extending about a cylindrical-shape of the plunger assembly 850, the plunger assembly 850 is directed in a rotatable direction (e.g., counterclockwise, clockwise, etc.) from a first orientation to a second orientation (See FIG. 38B). It should be understood that a configuration and length of the curved track 857 corresponds to a predetermined translation distance that the plunger assembly 850 undergoes relative to the vial body 589. The predetermined translation distance further corresponds to a minimum threshold volume of fluid medium (e.g. saline) that is to be drawn into the internal chamber 588 in response to a linear displacement of the plunger assembly 850 therein.

Accordingly, translating the fixed pin from a top portion of the curved track 857 to a bottom portion facilitates an extraction of the predetermined minimum volume threshold of fluid medium into the internal chamber 588 prior to a dose delivery by the delivery device as the plunger assembly 850 translates upward. The predetermined minimum volume threshold may comprise various suitable quantities for creating a suitable mixture of the therapeutic particles and the fluid medium (e.g. saline) therein (e.g. 10 milliliters) to ensure the resulting suspension fluid to be delivered is adequate for administration into a patient.

Referring now to FIG. 38B, once the fixed pin has slidably moved to a terminal end of the curved track 857 and the plunger assembly 850 has translated upward relative to the vial body 589 by the predetermined distance, the fixed pin is slidably received within the linear track 858 of the plunger assembly 850. The linear track 858 is in connection with the curved track 857 and extends parallel to a longitudinal length of the plunger assembly 850. In addition to a relocation of the fixed pin within the linear track 858, moving the fixed pin through the curved track 857 provides for a rotation of the lower engagement head 856 to the second orientation due to a simultaneous rotation of the plunger assembly 850. In this instance, due to the shape of the lower engagement head 856, the lower engagement head 856 provides a larger lateral width positioned beneath the pair of lever arms 522. Accordingly, actuation of the vial engagement mechanism 520 in a downward direction causes a disengagement of the pair of lever arms 522 with the bottom surface 853 of the upper engagement head 851 and a subsequent engagement with the top surface 855 of the lower engagement head 856.

It should be understood that actuation of the vial engagement mechanism 520 in a downward direction prior rotating the lower engagement head 856 to the second orientation will not provide a corresponding downward translation of the plunger assembly 850. In particular, a lateral width formed beneath the pair of lever arms 522 by the top surface 855 of the lower engagement head 856 is less than a width of the pair of lever arms 522 such that downward translation of the vial engagement mechanism 520 causes the pair of lever arms 522 to pass by the lower engagement head 856.

Still referring to FIG. 38B, with the plunger assembly 850 rotated to the second orientation, continued actuation of the vial engagement mechanism 520 provides for a translation of the pair of lever arms 522 and the plunger assembly 850 due to an engagement of the top surface 855 of the lower engagement head 856 with the pair of lever arms 522. In this instance, the fixed pin of the vial body 589 translates downward through the linear track 858, a vertical position of which is fixedly to the vial body 589 such that the plunger assembly 850 moves relative to the vial body 589 to deliver a dose therefrom. It should be understood that the plunger assembly 850 maintains a fixed orientation relative to the vial body 589 when the fixed pin of the vial body 589 translates downward through the linear track 858.

In other embodiments, the curved track 857 and the linear track 858 may be formed within the vial body 589 and/or the locking feature 586 of the vial assembly 580 such that the plunger assembly 850 includes the fixed pin extending laterally outward therefrom. In this instance, the plunger assembly 850 translates and rotates in a substantially similar manner as that described and shown herein as the fixed pin of the plunger assembly 850 travels along a travel path formed by the curved track of the vial assembly 580 prior to reaching a connection with the linear track of the vial assembly 580. In this embodiment, a length and geometry of the curved track and/or the linear track of the vial assembly 580 may be substantially similar to the configuration of the tracks 857, 858 shown and described herein.

### IIX. Suspension Chamber Vial Assembly

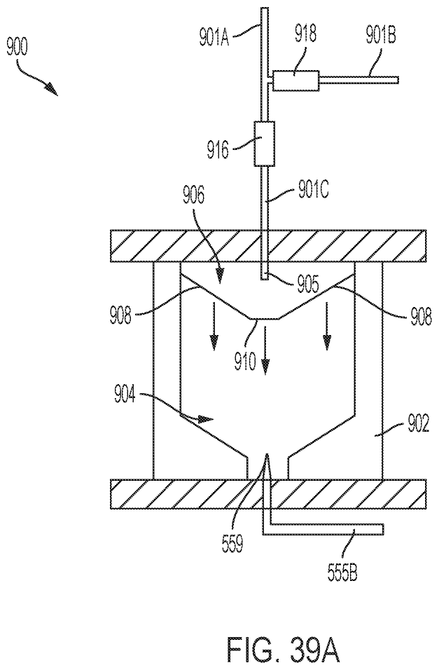

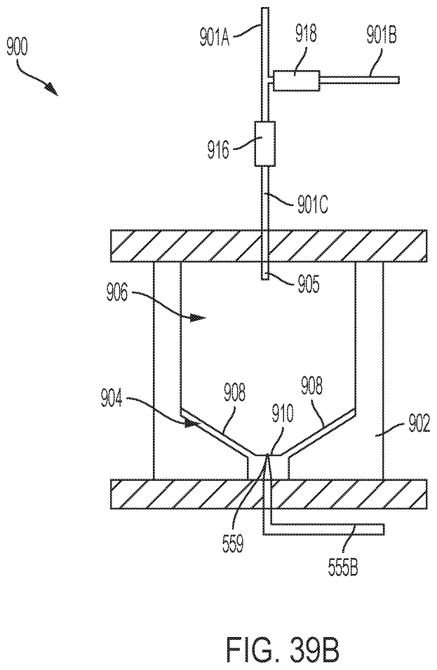

Referring now to FIGS. 39A-39B, an alternative vial assembly 900 is depicted. In the example shown and described herein, it should be understood that the vial assembly 900 is configured and operable just like the vial assembly 580 described above except for the differences explicitly noted herein. Accordingly, the vial assembly 900 of the present example may be readily incorporated into the sled assembly 540 described above. It should further be understood that the vial assembly 900, in many respects, functions substantially similar to the vial assembly 580 described above such that a version of the sled assembly 540 that is equipped with the vial assembly 900 of the present example may be configured and operable similar to the sled assembly 540 described above with the vial assembly 580 received therein except for the differences described below.

Although not shown, it should be understood that the vial assembly 900 may include a locking feature disposed along a top end of the vial assembly 900 that is substantially similar to the locking feature 586 of the vial assembly 580 shown and described above. Accordingly, the vial assembly 900 of the present example is configured to be received in, and securely couple with, the sled assembly 540 via an interlocking engagement between the locking feature of the vial assembly 900 and the locking system 550 of the sled assembly 540.

Specifically referring to FIG. 39A, the vial assembly 900 comprises a vial body 902 defining an inner chamber 904 with a pair of stoppers 908 and a floating septum 910 positioned therein. In particular, the pair of stoppers 908 and the floating septum 910 are disposed within the vial body 902 and are translatable with the inner chamber 904 in response to the vial assembly 900 receiving one or more fluid mediums therein. The pair of stoppers 908 are integrally formed with the floating septum 910, and more specifically extend laterally outward therefrom at opposing ends of the floating septum 910. The pair of pair of stoppers 908 are movably coupled to edges of the vial body 902 such that the pair of stoppers 908 are translatable thereon. With the floating septum 910 secured to the pair of stoppers 908, translation of the pair of stoppers 908 within the vial body 902 provides for a simultaneous translation of the floating septum 910 in the inner chamber 904.

It should be understood that the pair of pair of stoppers 908 are configured and operable similar to the stopper 594 of the vial assembly 580 shown and described above. Accordingly, the pair of stoppers 908 are configured to form a liquid-seal against the vial body 902 and are formed of various polymers with a predetermined viscoelasticity. For example, in some embodiments the stoppers 908 are formed of an elastomer, silicone, rubber, urethane, plastic, polyethylene, polypropylene, and/or the like. In this instance, the stoppers 908 are operable to inhibit a fluid media stored within the vial body 902 from extending (i.e., leaking) past the stoppers 908 and out of the vial body 902. Further, the floating septum 910 is configured and operable similar to the septum 592 of the vial assembly 580 shown and described above. The septum 910 forms a seal against a terminal end of the vial body 902. The septum 910 may be formed of various materials, including, for example, an elastomer, silicon, bromobutyl elastomer, rubber, urethanes, and/or the like. The septum 910 is configured to provide an air-tight seal for the vial body 902 to thereby inhibit a release of a fluid media stored therein (e.g., radioembolizing beads). As will be described in greater detail herein, the septum 910 of the vial assembly 900 is configured to be punctured by the needle 559 of the sled assembly 540 when the vial assembly 900 is received within the vial chamber 558, thereby establishing fluid communication between the vial body 902 and the sled assembly 540.

Still referring to FIG. 39A, in an exemplary mode of operation of the vial assembly 900 with the sled assembly 540, the vial body 902 of the vial assembly 900 is slidably received within the vial chamber 558 of the sled assembly 540 and a locking feature (not shown) of the vial assembly 900 securely fastens the vial body 902 therein in response to engaging the locking system 550 of the sled assembly 540. As briefly noted above, it should be understood that a locking feature of the vial assembly 900 may be configured and operable substantially similar to the locking feature 586 of the vial assembly 580 shown and described above.

A delivery line 901A is fluidly coupled to an external device, such as, for example, a syringe. Another delivery line 901B is fluidly coupled to the delivery line 901A via a once-way check valve 918 and to another external device, such as, for example, a bag containing a fluid medium therein (e.g. saline). It should be understood that the one-way check valve 918 is configured to permit fluid communication from the delivery line 901B to the delivery line 901A and simultaneously inhibit fluid communication from the delivery line 901A to the delivery line 901B. In this instance, the syringe is actuated to withdraw a fluid medium from the bag via the connection between the pair of delivery lines 901A, 901B and through the one-way check valve 918. With the syringe filled with the fluid medium therein, subsequent actuation of the syringe provides for a delivery of fluid medium to the vial assembly 900 via a delivery line 901C fluidly coupled to the syringe via a one-way check valve 916. Similar to the valve 918 described above, the one-way check valve 916 is configured to permit fluid communication from the delivery line 901A to the delivery line 901C and simultaneously inhibit fluid communication from the delivery line 901C to the delivery line 901A.

Still referring to FIG. 39A, it should be understood that the pair of stoppers 908 and the floating septum 910 are positioned along an upper region of the inner chamber 904 of the vial body 902 in a default position prior to the syringe delivering fluid medium thereto via the delivery line 901C. In this instance, the inner chamber 904 of the vial body 902 includes therapeutic particles preloaded therein such a volume of the therapeutic particles is determinative of a relative position of the pair of stoppers 908 and the floating septum 910 within the vial body 902. As the syringe is actuated and the fluid medium stored therein is delivered through the delivery lines 901A, 901C, the fluid medium is received within the inner chamber 904 of the vial body 902 via an inlet port 905 disposed within the inner chamber 904. In particular, the inlet port 905 is positioned relatively above a location of the pair of stoppers 908 and the floating septum 910 such that the inlet port 905 is separated from fluid communication with the needle 559 of the sled assembly 540 by the stoppers 908 and the floating septum 910 located therebetween.

Referring now to FIG. 39B, as the fluid medium is received within the inner chamber 904 of the vial body 902 and mixed with the therapeutic particles preloaded in the vial body 902, a volume of fluid in the inner chamber 904 is increased. In this instance, a pressure within the vial body 902 is increased and a force generated against the pair of stoppers 908 and the floating septum 910 causes the stoppers 908 and the floating septum 910 to translate within the vial body 902. In particular, the stoppers 908 and the floating septum 910 are linearly displaced away from the inlet port 905 such that the pair of stoppers 908 and the floating septum 910 translate toward the needle 559 as the fluid volume in the inner chamber 904 increases. Upon the vial body 902 receiving a predetermined volume of fluid therein, the floating septum 910 translates a corresponding linear distance within the inner chamber 904 to thereby encounter the needle 559. In this instance, the needle 559 punctures the floating septum 910 and the proximal manifold 555B of the sled assembly 540 establishes fluid communication with the fluid stored within the vial body 902 through the needle 559.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

For the purposes of describing and defining the present invention it is noted that the term “substantially” is used herein to represent the inherent degree of uncertainty that may be attributed to any quantitative comparison, value, measurement, or other representation. The term “substantially” is used herein also to represent the degree by which a quantitative representation may vary from a stated reference without resulting in a change in the basic function of the subject matter at issue. As such, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something slightly less than exact.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A delivery assembly, comprising:
a console including a vial containment region;
a vial engagement mechanism extending from the console within the vial containment region, wherein the engagement mechanism is configured to engage a vial assembly;
a sled assembly removably couplable to the console at the vial containment region; and
a safety shield removably couplable to the console over the vial containment region such that the vial engagement mechanism and the sled assembly are encapsulated within the safety shield when the safety shield is coupled thereto;
wherein the sled assembly, the vial assembly, and the safety shield are configured to inhibit radioactive emissions from within the vial containment region.

2. The delivery assembly of claim 1, wherein the vial assembly comprises:
a vial body formed of a material that inhibits radioactive emissions from within the vial body;
a locking feature coupled to the vial body and configured to securely engage the vial assembly to the sled assembly when the vial body is received therein; and
a plunger slidably translatable relative to the locking feature and through the vial body in response to the vial engagement mechanism engaging the vial assembly.

3. The delivery assembly of claim 2, wherein the plunger includes an engagement head having a pair of resilient arms that are sized and shaped to interlock with a pair of lever arms of the vial engagement mechanism.

4. The delivery assembly of claim 2, wherein the vial assembly comprises a septum disposed within a distal end of the vial body that is configured to seal the vial body.

5. The delivery assembly of any preceding claim, wherein the console comprises an interface display and one or more of a dosimeter, a linear encoder, an optical sensor, a linear displacement sensor, a flow sensor, an ultrasonic sensor, a magnetic encoder, a laser distance sensor, an inductance sensor, a radial encoder, a volumetric sensor, a radiation sensor, and a mechanical transducer.

6. The delivery assembly of claim 5, wherein the interface display is communicatively coupled to one or more of the dosimeter, linear encoder, optical sensor, linear displacement sensor, flow sensor, ultrasonic sensor, magnetic encoder, laser distance sensor, inductance sensor, radial encoder, volumetric sensor, radiation sensor, and mechanical transducers.

7. The delivery assembly of any preceding claim, wherein the vial engagement mechanism is disposed within the vial containment region and proximate to the sled assembly when the sled assembly is coupled to the console.

8. The delivery assembly of claim 7, further comprising a delivery handle coupled to the vial engagement mechanism and configured to move the vial engagement mechanism within the vial containment region.

9. The delivery assembly of claim 8, wherein the delivery handle is configured to move the vial assembly relative to the sled assembly in response to actuating the delivery handle.

10. The delivery assembly of any preceding claim, wherein the sled assembly comprises a locking system and a vial chamber, wherein the locking system forms an aperture sized and shaped to receive the vial assembly therethrough and into the vial chamber.

11. The delivery assembly of claim 10, wherein the locking system comprises annular projections extending laterally into the aperture, wherein the annular projections are sized and shaped to engage a locking feature of the vial assembly when the vial assembly is received through the aperture and into the vial chamber.

12. The delivery assembly of claim 10, wherein the vial chamber is encapsulated in a protective shield that is formed of a material that inhibits radioactive emissions from within the vial chamber.

13. The delivery assembly of claim 10, wherein the sled assembly comprises a needle extending within the vial chamber, the needle being configured to puncture a septum of the vial assembly when the vial assembly is received within the vial chamber through the aperture.

14. The delivery assembly of claim 13, wherein sled assembly further comprises a manifold fluidically coupled to the needle such that the manifold is in fluidic communication with the vial chamber via the needle.

15. The delivery assembly of claim 14, wherein the sled assembly comprises one or more ports that are in fluid communication with the vial chamber through the manifold, wherein one or more delivery lines are operable to couple to the manifold via the one or more ports.

16. The delivery assembly of any preceding claim, wherein the sled assembly, the vial assembly, and the safety shield are formed of materials that inhibit radioactive emissions from within the vial containment region.

17. The delivery assembly of claim 2, wherein the plunger includes an engagement head having a bottom surface that is sized and shaped to interlock with a pair of lever arms of the vial engagement mechanism.

18. The delivery assembly of claim 17, wherein the vial assembly further includes a pair of flexible wings rotatably coupled to the plunger such that the pair of flexible wings are configured to flexibly deform in response to translation of the plunger outwardly from the vial body.

19. The delivery assembly of claim 18, wherein the pair of flexible wings are configured to form an engagement surface that is sized and shaped to interlock with the pair of lever arms in response to a deformation of the pair of flexible wings as the plunger translates outwardly from the vial body.

20. The delivery assembly of claim 2, wherein the plunger includes an upper engagement head and a lower engagement head positioned relatively beneath the upper engagement head along a longitudinal length of the plunger.

21. The delivery assembly of claim 20, wherein the upper engagement head includes a bottom surface that is sized and shaped to interlock with a pair of lever arms of the vial engagement mechanism in response to an upward translation of the vial engagement mechanism.

22. The delivery assembly of claim 21, wherein the lower engagement head includes a top surface that is sized and shaped to interlock with the pair of lever arms of the vial engagement mechanism in response to a downward translation of the vial engagement mechanism.

23. The delivery assembly of claim 22, wherein the plunger further includes at least a curved track and a linear track disposed along an exterior surface of the plunger, wherein the curved track and the linear track are sized and shaped to slidably receive a fixed pin of the vial body therein.

24. The delivery assembly of claim 23, wherein the curved track extends around the exterior surface of the plunger and the linear track extends along the exterior surface and parallel to a longitudinal length of the plunger.

25. The delivery assembly of claim 24, wherein the plunger is configured to rotate within and translate outwardly from the vial body in response to the pair of lever arms interlocking with the bottom surface of the upper engagement head when the vial engagement mechanism translates upward due to the fixed pin being received within the curved track.

26. The delivery assembly of claim 24, wherein the plunger is configured to translate into the vial body at a fixed orientation relative thereto in response to the pair of lever arms interlocking with the top surface of the lower engagement head when the vial engagement mechanism translates downward due to the fixed pin being received within the linear track.

27. The delivery assembly of any preceding claim, wherein the vial assembly comprises an inner chamber and a floating septum movably disposed therein.

28. The delivery assembly of claim 27, wherein the floating septum is configured to translate within the vial body in response to the inner chamber receiving fluid medium therein.

29. The delivery assembly of claim 28, wherein the floating septum engages a needle of the sled assembly positioned at a bottom end of the inner chamber as the floating septum translates within the vial body and the fluid medium within the inner chamber increases.

30. The delivery device of any preceding claim, wherein the vial engagement mechanism is coupled to an electrical system such that translation of the vial engagement mechanism within the vial containment region is electrically-driven.

31. The delivery device of any preceding claim, wherein the sled assembly includes a battery that is configured to supply power to the sled assembly and the console in response to the sled assembly coupling with the console at the vial containment region.

32. The delivery device of claim 31, wherein the sled assembly includes a battery status indicator communicatively coupled to the battery and configured to display data indicative of a charge of the battery.

33. The delivery device of any preceding claim, wherein the sled assembly is configured to maintain fluid leaks of therapeutic particles therein such that the sled assembly is operable to inhibit radioactive emissions and fluid leaks from the therapeutic particles stored therein.

34. A treatment delivery device, comprising:
a housing having a protective shielding material, wherein the housing is sized and shaped to couple to an instrument;
a vial compartment disposed within the housing, wherein the vial compartment is configured to receive a therapeutic media therein, and wherein the protective shielding material inhibits exposure of the therapeutic media external from the housing;
a fluid reservoir disposed within the housing, wherein the fluid reservoir is configured to receive a fluid medium therein, and wherein the fluid reservoir is in fluidic communication with the vial compartment; and
an actuator movably coupled to the housing such that moving the actuator delivers the fluid medium to the vial compartment;
wherein the actuator is configured to deliver a mixture of the fluid medium and the therapeutic media to the instrument.

35. A treatment delivery device, comprising:
a base plate;
a housing coupled to the base plate, the housing defining a compartment for storing therapeutic substances, the housing formed of a material configured to inhibit radioactive emissions from the compartment;
a reservoir coupled to the base plate, the reservoir defining a storage for storing fluid substances; and
a handle assembly coupled to the base plate, the handle assembly is movably coupled to the housing, wherein the handle assembly is configured to generate a negative pressure within the housing and the reservoir such that moving the handle assembly suctions the fluid substances from the reservoir and the therapeutic substances from the housing.

36. A handheld delivery device, comprising:
a first chamber sized and shaped to receive a first device including therapeutic particles stored therein;
a second chamber sized and shaped to receive a second device including fluid medium stored therein;
a distal end including a manifold and a needle disposed therein, the distal end is coupled to the first chamber and the second chamber such that the manifold is in fluid communication with the first device and the second device, wherein the needle extends into the first chamber and coupled to the first device received within the first chamber,
wherein the therapeutic particles stored within the first device and the fluid medium stored within the second device are received at the manifold thereby forming a mixture therein.

37. The handheld delivery device of any preceding claim, wherein the first device is punctured by the needle when received within the first chamber.

38. The handheld delivery device of any preceding claim, wherein the first device is a vial assembly including an inner chamber for storing the therapeutic particles therein and a protective shield disposed about the inner chamber for inhibiting radioactive emissions generated by the therapeutic particles.

39. The handheld delivery device of claim 38, wherein the vial assembly includes a handle and a plunger coupled to the inner chamber such that translation of the plunger into the inner chamber delivers the therapeutic particles into the manifold via the needle in response to actuation of the handle.

40. The handheld delivery device of claim 39, wherein the handle is configured to translate the plunger in response to a rotation of the handle.

41. The handheld delivery device of claim 39, wherein the handle is configured to translate the plunger in response to a translation of the handle.

42. The handheld delivery device of claim 39, further comprising a safety tab removably coupled to the vial assembly and configured to inhibit translation of the plunger thereby preventing delivery of the therapeutic particles into the manifold.

43. The handheld delivery device of claim 39, wherein the safety tab is configured to be decoupled from the vial assembly to permit translation of the plunger in response to applying a force thereto.

44. The handheld delivery device of any preceding claim, wherein the first device includes a retention mechanism and the first chamber includes a corresponding retention mechanism such that the first device is securely fastened to the first chamber in response to the retention mechanism of the first device coupling the corresponding retention mechanism of the first chamber.

45. The handheld delivery device of claim 44, wherein the retention mechanism of the first device comprises a depressible button and the corresponding retention mechanism of the first chamber is an aperture sized and shaped to receive the depressible button therethrough.

46. The handheld delivery device of any preceding claim, further comprising a catheter hub disposed within the distal end and in fluid communication with the manifold, wherein the catheter hub is configured to couple the manifold to an external device such that the external device is in fluid communication with the mixture.

47. The handheld delivery device of any preceding claim, wherein the second device is a fluid reservoir and the fluid medium stored therein is saline or saline-contrast mixture.

48. The handheld delivery device of any preceding claim, further comprising one or more of a dosimeter, a linear encoder, an optical sensor, a linear displacement sensor, a flow sensor, an ultrasonic sensor, a magnetic encoder, a laser distance sensor, an inductance sensor, a radial encoder, volumetric sensor, and a mechanical transducer.

49. The handheld delivery device of claim 48, further comprising one or more display outputs communicatively coupled to one or more of the dosimeter, linear encoder, optical sensor, linear displacement sensor, flow sensor, ultrasonic sensor, magnetic encoder, laser distance sensor, inductance sensor, radial encoder, volumetric sensor, and mechanical transducer.

50. The handheld delivery device of claim 48, further comprising a remote display communicatively coupled to one or more of the dosimeter, linear encoder, optical sensor, linear displacement sensor, flow sensor, ultrasonic sensor, magnetic encoder, laser distance sensor, inductance sensor, radial encoder, volumetric sensor, and mechanical transducer.

51. The handheld delivery device of claim 50, wherein the remote display comprises a smart device, a tablet, or a computer.

52. A handheld delivery device, comprising:
a housing including a manifold disposed therein;
a first device disposed within the housing and storing a first fluid medium, wherein the first device is in fluid communication with the manifold;
a second device disposed within the housing and storing a second fluid medium, wherein the second device is in fluid communication with the manifold;
a syringe at least partially disposed within the housing, the syringe including:
an external chamber,
an internal chamber disposed within the external chamber and storing therapeutic particles therein, and
a needle disposed within the external chamber and in fluid communication with the manifold such that the external chamber is fluidly coupled to the manifold via the needle,
wherein:
the internal chamber is configured to translate within the external chamber and engage the needle such that the internal chamber fluidly couples to the manifold when engaged with the needle; and
the therapeutic particles, the first fluid medium, and the second fluid medium are received at the manifold thereby forming a mixture therein.

53. The handheld delivery device of any preceding claim, further comprising one or more of a dosimeter, a linear encoder, an optical sensor, a linear displacement sensor, a flow sensor, an ultrasonic sensor, a magnetic encoder, a laser distance sensor, an inductance sensor, a radial encoder, volumetric sensor, and a mechanical transducer.

54. The handheld delivery device of claim 53, further comprising one or more display outputs communicatively coupled to one or more of the dosimeter, linear encoder, optical sensor, linear displacement sensor, flow sensor, ultrasonic sensor, magnetic encoder, laser distance sensor, inductance sensor, radial encoder, volumetric sensor, and mechanical transducer.

55. The handheld delivery device of claim 53, further comprising a remote display communicatively coupled to one or more of the dosimeter, linear encoder, optical sensor, linear displacement sensor, flow sensor, ultrasonic sensor, magnetic encoder, laser distance sensor, inductance sensor, radial encoder, volumetric sensor, and mechanical transducer.

56. The handheld delivery device of claim 55, wherein the remote display comprises a smart device, a tablet, or a computer.

57. The handheld delivery device of any preceding claim, wherein the syringe includes a handle extending outwardly from the housing such that the handle is accessible externally from the housing.

58. The handheld delivery device of any preceding claim, wherein the internal chamber is configured to translate within the external chamber and engage the needle in response to actuation of the handle external of the housing.

59. The handheld delivery device of any preceding claim, wherein the housing includes a first switch communicatively coupled to the first device such that actuation of the first switch automates delivery of the first fluid medium to the manifold.

60. The handheld delivery device of claim 59, wherein the housing includes a second switch communicatively coupled to the second device such that actuation of the second switch automates delivery of the second fluid medium to the manifold.

61. The handheld delivery device of claim 60, wherein the housing includes a third switch communicatively coupled to the syringe such that actuation of the third switch translates the internal chamber within the external chamber.

62. A sterile container assembly comprising:
a top housing including a closed end and an open end, wherein the closed end of the top housing includes material configured to form a liquid seal therein and the open end of the top housing includes a top mating system;
a bottom housing including a closed end and an open end, wherein the closed end of the bottom housing includes material configured to form a liquid seal therein and the open end of the bottom housing includes a bottom mating system;
wherein:
the top housing and the bottom housing are sized and shaped to receive a device therein when the open end of the top housing couples to the open end of the bottom housing via the top mating system engaging the bottom mating system; and
a gasket seal is formed between the open end of the top housing and the open end of the bottom housing in response to the top mating system engaging the bottom mating system.

63. The sterile container assembly of claim 62, wherein the top housing and the bottom housing are configured to inhibit leaks of therapeutic particles externally therefrom when the gasket seal is formed between the open end of the top housing and the open end of the bottom housing.

64. The sterile container assembly of claim 62, wherein the closed end of the top housing and the closed end of the bottom housing are configured to facilitate steam penetration through the liquid seals formed thereon when the open end of the top housing is coupled to the open end of the bottom housing.
